SEQUENCE LISTING (44 sequences)

SEQ ID NO: 1 - protein, 198 aa, Barley stripe mosaic virus
MPNVSLTAKGGGHYIEDQWDTQVVEAGVFDDWWVHVEAWNKFLDNLRGINFSVASSRSQVAEYLAALDRDLPADVDRRFAGARGQIGSPNYLPAPKFFRLDKRTIAELTRLSRLTDQPHNNRDIELNRAKRATTNPSPPAQAPSENLTLRDVQPLKDSALHYQYVLIDLQSARLPVYTRKTFERELALEWIIPDAEEA

SEQ ID NO: 2 - protein, 198 aa, synthetic construct
  source 1..198: note = BSMV with a point mutation
  SITE 68: note = point mutation at residue D68N
MPNVSLTAKGGGHYIEDQWDTQVVEAGVFDDWWVHVEAWNKFLDNLRGINFSVASSRSQVAEYLAALNRDLPADVDRRFAGARGQIGSPNYLPAPKFFRLDKRTIAELTRLSRLTDQPHNNRDIELNRAKRATTNPSPPAQAPSENLTLRDVQPLKDSALHYQYVLIDLQSARLPVYTRKTFERELALEWIIPDAEEA

SEQ ID NO: 3 - protein, 198 aa, synthetic construct
  source 1..198: note = BSMV with a point mutation
  SITE 70: note = point mutation at residue D70N
MPNVSLTAKGGGHYIEDQWDTQVVEAGVFDDWWVHVEAWNKFLDNLRGINFSVASSRSQVAEYLAALDRNLPADVDRRFAGARGQIGSPNYLPAPKFFRLDKRTIAELTRLSRLTDQPHNNRDIELNRAKRATTNPSPPAQAPSENLTLRDVQPLKDSALHYQYVLIDLQSARLPVYTRKTFERELALEWIIPDAEEA

SEQ ID NO: 4 - protein, 198 aa, synthetic construct
  source 1..198: note = BSMV with a point mutation
  SITE 101: note = point mutation at residue D101K
MPNVSLTAKGGGHYIEDQWDTQVVEAGVFDDWWVHVEAWNKFLDNLRGINFSVASSRSQVAEYLAALDRDLPADVDRRFAGARGQIGSPNYLPAPKFFRLKKRTIAELTRLSRLTDQPHNNRDIELNRAKRATTNPSPPAQAPSENLTLRDVQPLKDSALHYQYVLIDLQSARLPVYTRKTFERELALEWIIPDAEEA

SEQ ID NO: 5 - protein, 198 aa, synthetic construct
  source 1..198: note = BSMV with a point mutation
  SITE 101: note = point mutation at residue D101N
MPNVSLTAKGGGHYIEDQWDTQVVEAGVFDDWWVHVEAWNKFLDNLRGINFSVASSRSQVAEYLAALDRDLPADVDRRFAGARGQIGSPNYLPAPKFFRLNKRTIAELTRLSRLTDQPHNNRDIELNRAKRATTNPSPPAQAPSENLTLRDVQPLKDSALHYQYVLIDLQSARLPVYTRKTFERELALEWIIPDAEEA

SEQ ID NO: 6 - protein, 198 aa, synthetic construct
  source 1..198: note = BSMV with a point mutation
  SITE 101: note = point mutation at residue D101R
MPNVSLTAKGGGHYIEDQWDTQVVEAGVFDDWWVHVEAWNKFLDNLRGINFSVASSRSQVAEYLAALDRDLPADVDRRFAGARGQIGSPNYLPAPKFFRLRKRTIAELTRLSRLTDQPHNNRDIELNRAKRATTNPSPPAQAPSENLTLRDVQPLKDSALHYQYVLIDLQSARLPVYTRKTFERELALEWIIPDAEEA

SEQ ID NO: 7 - protein, 198 aa, synthetic construct
  source 1..198: note = BSMV with a point mutation
  SITE 37: note = point mutation at residue E37Q
MPNVSLTAKGGGHYIEDQWDTQVVEAGVFDDWWVHVQAWNKFLDNLRGINFSVASSRSQVAEYLAALDRDLPADVDRRFAGARGQIGSPNYLPAPKFFRLDKRTIAELTRLSRLTDQPHNNRDIELNRAKRATTNPSPPAQAPSENLTLRDVQPLKDSALHYQYVLIDLQSARLPVYTRKTFERELALEWIIPDAEEA

SEQ ID NO: 8 - protein, 198 aa, synthetic construct
  source 1..198: note = BSMV with a point mutation
  SITE 37: note = point mutation at residue E37R
MPNVSLTAKGGGHYIEDQWDTQVVEAGVFDDWWVHVRAWNKFLDNLRGINFSVASSRSQVAEYLAALDRDLPADVDRRFAGARGQIGSPNYLPAPKFFRLDKRTIAELTRLSRLTDQPHNNRDIELNRAKRATTNPSPPAQAPSENLTLRDVQPLKDSALHYQYVLIDLQSARLPVYTRKTFERELALEWIIPDAEEA

SEQ ID NO: 9 - protein, 198 aa, synthetic construct
  source 1..198: note = BSMV with a point mutation
  SITE 62: note = point mutation at residue E62Q
MPNVSLTAKGGGHYIEDQWDTQVVEAGVFDDWWVHVEAWNKFLDNLRGINFSVASSRSQVAQYLAALDRDLPADVDRRFAGARGQIGSPNYLPAPKFFRLDKRTIAELTRLSRLTDQPHNNRDIELNRAKRATTNPSPPAQAPSENLTLRDVQPLKDSALHYQYVLIDLQSARLPVYTRKTFERELALEWIIPDAEEA

SEQ ID NO: 10 - protein, 198 aa, synthetic construct
  source 1..198: note = BSMV with two point mutations
  SITE 62: note = point mutation at residue E62Q
  SITE 101: note = point mutation at residue D101N
MPNVSLTAKGGGHYIEDQWDTQVVEAGVFDDWWVHVEAWNKFLDNLRGINFSVASSRSQVAQYLAALDRDLPADVDRRFAGARGQIGSPNYLPAPKFFRLNKRTIAELTRLSRLTDQPHNNRDIELNRAKRATTNPSPPAQAPSENLTLRDVQPLKDSALHYQYVLIDLQSARLPVYTRKTFERELALEWIIPDAEEA

SEQ ID NO: 11 - DNA, 189 bp, Tobacco mosaic virus
  source 1..189: mol_type = genomic DNA
gtattgtttatagaaataatataaaattaggtttgagagagaagattacaaacgtgagagacggagggcccatggaacttacagaagaagtcgttgatgagttcatggaagatgtccctatgtcgatcaggcttgcaaagtttcgatctcgaaccggaaaaaagagtgatgtccgcaaagggaaaaata

SEQ ID NO: 12 - DNA, 597 bp, Barley stripe mosaic virus
  source 1..597: mol_type = genomic DNA
atgcccaacgtatcactgacagcgaaagggggaggtcattacatcgaagatcagtgggatacgcaagtcgtcgaagcaggagtgttcgacgactggtgggtgcatgtagaggcctggaataagtttcttgacaatctgcgcggcatcaatttttccgtcgccagcagtcgctcacaagtagcagagtatttggctgctttggatcgtgaccttccggctgacgttgatcgtcgtttcgcgggtgcacgtggtcaaatcggcagccccaattacttaccagcacctaaattttttcgtcttgataaacgtacaatcgctgaattgacacgtttgtcgcgcttgacggatcagccccacaacaatcgtgatatcgagttaaatcgcgcaaaacgcgcaacaacaaatcctagcccacctgctcaagccccgagcgaaaaccttacactgcgcgacgtgcaacccttaaaggactccgcgttacattatcagtatgtccttattgatcttcagtccgcacgcttgcctgtgtatacccgcaagactttcgagcgcgagctggctctggagtggatcattccagatgcagaggaagcataa

SEQ ID NO: 13 - DNA, 661 bp, synthetic construct
  source 1..661: mol_type = other DNA; note = Linker region
accggttttaaatatgtcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgaaacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaacttgatagtccggacctgcaggacgcgtgtcgacgtttgagagagaagattacaaacgtgagagacggagggcccatggaacttacagaagaagtcgttgatgagttcatggaagatgtccctatgtcgatcaggcttgcaaagtttcgatctcgaaccggcctaggctcgtg

SEQ ID NO: 14 - DNA, 786 bp, synthetic construct
  source 1..786: mol_type = other DNA; note = Fusion of barley stripe mosiac virus capsid protein and origin of assembly from tobacco mosaic virus
atgcccaacgtatcactgacagcgaaagggggaggtcattacatcgaagatcagtgggatacgcaagtcgtcgaagcaggagtgttcgacgactggtgggtgcatgtagaggcctggaataagtttcttgacaatctgcgcggcatcaatttttccgtcgccagcagtcgctcacaagtagcagagtatttggctgctttggatcgtgaccttccggctgacgttgatcgtcgtttcgcgggtgcacgtggtcaaatcggcagccccaattacttaccagcacctaaattttttcgtcttgataaacgtacaatcgctgaattgacacgtttgtcgcgcttgacggatcagccccacaacaatcgtgatatcgagttaaatcgcgcaaaacgcgcaacaacaaatcctagcccacctgctcaagccccgagcgaaaaccttacactgcgcgacgtgcaacccttaaaggactccgcgttacattatcagtatgtccttattgatcttcagtccgcacgcttgcctgtgtatacccgcaagactttcgagcgcgagctggctctggagtggatcattccagatgcagaggaagcataagtattgtttatagaaataatataaaattaggtttgagagagaagattacaaacgtgagagacggagggcccatggaacttacagaagaagtcgttgatgagttcatggaagatgtccctatgtcgatcaggcttgcaaagtttcgatctcgaaccggaaaaaagagtgatgtccgcaaagggaaaaata

SEQ ID NO: 15 - DNA, 1447 bp, synthetic construct
  source 1..1447: mol_type = other DNA; note = Fusion of BSMV-CP with a linker and a tobacco mosaic virus OAS
atgcccaacgtatcactgacagcgaaagggggaggtcattacatcgaagatcagtgggatacgcaagtcgtcgaagcaggagtgttcgacgactggtgggtgcatgtagaggcctggaataagtttcttgacaatctgcgcggcatcaatttttccgtcgccagcagtcgctcacaagtagcagagtatttggctgctttggatcgtgaccttccggctgacgttgatcgtcgtttcgcgggtgcacgtggtcaaatcggcagccccaattacttaccagcacctaaattttttcgtcttgataaacgtacaatcgctgaattgacacgtttgtcgcgcttgacggatcagccccacaacaatcgtgatatcgagttaaatcgcgcaaaacgcgcaacaacaaatcctagcccacctgctcaagccccgagcgaaaaccttacactgcgcgacgtgcaacccttaaaggactccgcgttacattatcagtatgtccttattgatcttcagtccgcacgcttgcctgtgtatacccgcaagactttcgagcgcgagctggctctggagtggatcattccagatgcagaggaagcataaaccggttttaaatatgtcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgaaacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaacttgatagtccggacctgcaggacgcgtgtcgacgtttgagagagaagattacaaacgtgagagacggagggcccatggaacttacagaagaagtcgttgatgagttcatggaagatgtccctatgtcgatcaggcttgcaaagtttcgatctcgaaccggcctaggctcgtggtattgtttatagaaataatataaaattaggtttgagagagaagattacaaacgtgagagacggagggcccatggaacttacagaagaagtcgttgatgagttcatggaagatgtccctatgtcgatcaggcttgcaaagtttcgatctcgaaccggaaaaaagagtgatgtccgcaaagggaaaaata

SEQ ID NO: 16 - DNA, 6560 bp, synthetic construct
  source 1..6560: mol_type = other DNA; note = pET21 plasmid vector carrying the sequence for BSMV-CP with a point mutation at residue D70N
tctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgatgcccaacgtatcactgacagcgaaagggggaggtcattacatcgaagatcagtgggatacgcaagtcgtcgaagcaggagtgttcgacgactggtgggtgcatgtagaggcctggaataagtttcttgacaatctgcgcggcatcaatttttccgtcgccagcagtcgctcacaagtagcagagtatttggctgctttggatcgtaaccttccggctgacgttgatcgtcgtttcgcgggtgcacgtggtcaaatcggcagccccaattacttaccagcacctaaattttttcgtcttgataaacgtacaatcgctgaattgacacgtttgtcgcgcttgacggatcagccccacaacaatcgtgatatcgagttaaatcgcgcaaaacgcgcaacaacaaatcctagcccacctgctcaagccccgagcgaaaaccttacactgcgcgacgtgcaacccttaaaggactccgcgttacattatcagtatgtccttattgatcttcagtccgcacgcttgcctgtgtatacccgcaagactttcgagcgcgagctggctctggagtggatcattccagatgcagaggaagcataaaccggttttaaatatgtcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgaaacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaacttgatagtccggacctgcaggacgcgtgtcgatcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaa

SEQ ID NO: 17 - DNA, 6560 bp, synthetic construct
  source 1..6560: mol_type = other DNA; note = pET21 plasmid vector carrying the sequence for BSMV-CP with a point mutation at the D68N residue
tctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgatgcccaacgtatcactgacagcgaaagggggaggtcattacatcgaagatcagtgggatacgcaagtcgtcgaagcaggagtgttcgacgactggtgggtgcatgtagaggcctggaataagtttcttgacaatctgcgcggcatcaatttttccgtcgccagcagtcgctcacaagtagcagagtatttggctgctttgaatcgtgaccttccggctgacgttgatcgtcgtttcgcgggtgcacgtggtcaaatcggcagccccaattacttaccagcacctaaattttttcgtcttgataaacgtacaatcgctgaattgacacgtttgtcgcgcttgacggatcagccccacaacaatcgtgatatcgagttaaatcgcgcaaaacgcgcaacaacaaatcctagcccacctgctcaagccccgagcgaaaaccttacactgcgcgacgtgcaacccttaaaggactccgcgttacattatcagtatgtccttattgatcttcagtccgcacgcttgcctgtgtatacccgcaagactttcgagcgcgagctggctctggagtggatcattccagatgcagaggaagcataaaccggttttaaatatgtcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgaaacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaacttgatagtccggacctgcaggacgcgtgtcgatcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaa

SEQ ID NO: 18 - DNA, 6560 bp, synthetic construct
  source 1..6560: mol_type = other DNA; note = pET21 plasmid vector carrying the sequence for BSMV-CP with a point mutation at residue D101K
tctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgatgcccaacgtatcactgacagcgaaagggggaggtcattacatcgaagatcagtgggatacgcaagtcgtcgaagcaggagtgttcgacgactggtgggtgcatgtagaggcctggaataagtttcttgacaatctgcgcggcatcaatttttccgtcgccagcagtcgctcacaagtagcagagtatttggctgctttggatcgtgaccttccggctgacgttgatcgtcgtttcgcgggtgcacgtggtcaaatcggcagccccaattacttaccagcacctaaattttttcgtcttaaaaaacgtacaatcgctgaattgacacgtttgtcgcgcttgacggatcagccccacaacaatcgtgatatcgagttaaatcgcgcaaaacgcgcaacaacaaatcctagcccacctgctcaagccccgagcgaaaaccttacactgcgcgacgtgcaacccttaaaggactccgcgttacattatcagtatgtccttattgatcttcagtccgcacgcttgcctgtgtatacccgcaagactttcgagcgcgagctggctctggagtggatcattccagatgcagaggaagcataaaccggttttaaatatgtcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgaaacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaacttgatagtccggacctgcaggacgcgtgtcgatcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaa

SEQ ID NO: 19 - DNA, 6560 bp, synthetic construct
  source 1..6560: mol_type = other DNA; note = pET21 plasmid vector carrying the sequence for BSMV-CP with a point mutation at residue D101R
tctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgatgcccaacgtatcactgacagcgaaagggggaggtcattacatcgaagatcagtgggatacgcaagtcgtcgaagcaggagtgttcgacgactggtgggtgcatgtagaggcctggaataagtttcttgacaatctgcgcggcatcaatttttccgtcgccagcagtcgctcacaagtagcagagtatttggctgctttggatcgtgaccttccggctgacgttgatcgtcgtttcgcgggtgcacgtggtcaaatcggcagccccaattacttaccagcacctaaattttttcgtcttcgtaaacgtacaatcgctgaattgacacgtttgtcgcgcttgacggatcagccccacaacaatcgtgatatcgagttaaatcgcgcaaaacgcgcaacaacaaatcctagcccacctgctcaagccccgagcgaaaaccttacactgcgcgacgtgcaacccttaaaggactccgcgttacattatcagtatgtccttattgatcttcagtccgcacgcttgcctgtgtatacccgcaagactttcgagcgcgagctggctctggagtggatcattccagatgcagaggaagcataaaccggttttaaatatgtcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgaaacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaacttgatagtccggacctgcaggacgcgtgtcgatcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaa

SEQ ID NO: 20 - DNA, 6560 bp, synthetic construct
  source 1..6560: mol_type = other DNA; note = pET21 plasmid vector carrying the sequence for BSMV-CP with a point mutation at residue E37Q
tctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgatgcccaacgtatcactgacagcgaaagggggaggtcattacatcgaagatcagtgggatacgcaagtcgtcgaagcaggagtgttcgacgactggtgggtgcatgtacaggcctggaataagtttcttgacaatctgcgcggcatcaatttttccgtcgccagcagtcgctcacaagtagcagagtatttggctgctttggatcgtgaccttccggctgacgttgatcgtcgtttcgcgggtgcacgtggtcaaatcggcagccccaattacttaccagcacctaaattttttcgtcttgataaacgtacaatcgctgaattgacacgtttgtcgcgcttgacggatcagccccacaacaatcgtgatatcgagttaaatcgcgcaaaacgcgcaacaacaaatcctagcccacctgctcaagccccgagcgaaaaccttacactgcgcgacgtgcaacccttaaaggactccgcgttacattatcagtatgtccttattgatcttcagtccgcacgcttgcctgtgtatacccgcaagactttcgagcgcgagctggctctggagtggatcattccagatgcagaggaagcataaaccggttttaaatatgtcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgaaacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaacttgatagtccggacctgcaggacgcgtgtcgatcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaa

SEQ ID NO: 21 - DNA, 6560 bp, synthetic construct
  source 1..6560: mol_type = other DNA; note = pET21 plasmid vector carrying the sequence for BSMV-CP with a point mutation at residue E37R
tctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgatgcccaacgtatcactgacagcgaaagggggaggtcattacatcgaagatcagtgggatacgcaagtcgtcgaagcaggagtgttcgacgactggtgggtgcatgtacgtgcctggaataagtttcttgacaatctgcgcggcatcaatttttccgtcgccagcagtcgctcacaagtagcagagtatttggctgctttggatcgtgaccttccggctgacgttgatcgtcgtttcgcgggtgcacgtggtcaaatcggcagccccaattacttaccagcacctaaattttttcgtcttgataaacgtacaatcgctgaattgacacgtttgtcgcgcttgacggatcagccccacaacaatcgtgatatcgagttaaatcgcgcaaaacgcgcaacaacaaatcctagcccacctgctcaagccccgagcgaaaaccttacactgcgcgacgtgcaacccttaaaggactccgcgttacattatcagtatgtccttattgatcttcagtccgcacgcttgcctgtgtatacccgcaagactttcgagcgcgagctggctctggagtggatcattccagatgcagaggaagcataaaccggttttaaatatgtcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgaaacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaacttgatagtccggacctgcaggacgcgtgtcgatcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaa

SEQ ID NO: 22 - DNA, 6560 bp, synthetic construct
  source 1..6560: mol_type = other DNA; note = pET21 plasmid vector carrying the sequence for BSMV-CP with a point mutation at residue D101N
tctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgatgcccaacgtatcactgacagcgaaagggggaggtcattacatcgaagatcagtgggatacgcaagtcgtcgaagcaggagtgttcgacgactggtgggtgcatgtagaggcctggaataagtttcttgacaatctgcgcggcatcaatttttccgtcgccagcagtcgctcacaagtagcagagtatttggctgctttggatcgtgaccttccggctgacgttgatcgtcgtttcgcgggtgcacgtggtcaaatcggcagccccaattacttaccagcacctaaattttttcgtcttaataaacgtacaatcgctgaattgacacgtttgtcgcgcttgacggatcagccccacaacaatcgtgatatcgagttaaatcgcgcaaaacgcgcaacaacaaatcctagcccacctgctcaagccccgagcgaaaaccttacactgcgcgacgtgcaacccttaaaggactccgcgttacattatcagtatgtccttattgatcttcagtccgcacgcttgcctgtgtatacccgcaagactttcgagcgcgagctggctctggagtggatcattccagatgcagaggaagcataaaccggttttaaatatgtcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgaaacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaacttgatagtccggacctgcaggacgcgtgtcgatcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaa

SEQ ID NO: 23 - DNA, 6560 bp, synthetic construct
  source 1..6560: mol_type = other DNA; note = pET21 plasmid vector carrying the sequence for BSMV-CP having a point mutation at residue E62Q
tctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgatgcccaacgtatcactgacagcgaaagggggaggtcattacatcgaagatcagtgggatacgcaagtcgtcgaagcaggagtgttcgacgactggtgggtgcatgtagaggcctggaataagtttcttgacaatctgcgcggcatcaatttttccgtcgccagcagtcgctcacaagtagcacagtatttggctgctttggatcgtgaccttccggctgacgttgatcgtcgtttcgcgggtgcacgtggtcaaatcggcagccccaattacttaccagcacctaaattttttcgtcttgataaacgtacaatcgctgaattgacacgtttgtcgcgcttgacggatcagccccacaacaatcgtgatatcgagttaaatcgcgcaaaacgcgcaacaacaaatcctagcccacctgctcaagccccgagcgaaaaccttacactgcgcgacgtgcaacccttaaaggactccgcgttacattatcagtatgtccttattgatcttcagtccgcacgcttgcctgtgtatacccgcaagactttcgagcgcgagctggctctggagtggatcattccagatgcagaggaagcataaaccggttttaaatatgtcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgaaacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaacttgatagtccggacctgcaggacgcgtgtcgatcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaa

SEQ ID NO: 24 - DNA, 6560 bp, synthetic construct
  source 1..6560: mol_type = other DNA; note = pET21 plasmid vector carrying the sequence for BSMV-CP having a point mutation at both residues E62Q and D101N
tctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgatgcccaacgtatcactgacagcgaaagggggaggtcattacatcgaagatcagtgggatacgcaagtcgtcgaagcaggagtgttcgacgactggtgggtgcatgtagaggcctggaataagtttcttgacaatctgcgcggcatcaatttttccgtcgccagcagtcgctcacaagtagcacagtatttggctgctttggatcgtgaccttccggctgacgttgatcgtcgtttcgcgggtgcacgtggtcaaatcggcagccccaattacttaccagcacctaaattttttcgtcttaataaacgtacaatcgctgaattgacacgtttgtcgcgcttgacggatcagccccacaacaatcgtgatatcgagttaaatcgcgcaaaacgcgcaacaacaaatcctagcccacctgctcaagccccgagcgaaaaccttacactgcgcgacgtgcaacccttaaaggactccgcgttacattatcagtatgtccttattgatcttcagtccgcacgcttgcctgtgtatacccgcaagactttcgagcgcgagctggctctggagtggatcattccagatgcagaggaagcataaaccggttttaaatatgtcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgaaacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaacttgatagtccggacctgcaggacgcgtgtcgatcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaa

SEQ ID NO: 25 - DNA, 6560 bp, synthetic construct
  source 1..6560: mol_type = other DNA; note = pET21 plasmid vector carrying the sequence for wild-type BSMV-CP
tctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgatgcccaacgtatcactgacagcgaaagggggaggtcattacatcgaagatcagtgggatacgcaagtcgtcgaagcaggagtgttcgacgactggtgggtgcatgtagaggcctggaataagtttcttgacaatctgcgcggcatcaatttttccgtcgccagcagtcgctcacaagtagcagagtatttggctgctttggatcgtgaccttccggctgacgttgatcgtcgtttcgcgggtgcacgtggtcaaatcggcagccccaattacttaccagcacctaaattttttcgtcttgataaacgtacaatcgctgaattgacacgtttgtcgcgcttgacggatcagccccacaacaatcgtgatatcgagttaaatcgcgcaaaacgcgcaacaacaaatcctagcccacctgctcaagccccgagcgaaaaccttacactgcgcgacgtgcaacccttaaaggactccgcgttacattatcagtatgtccttattgatcttcagtccgcacgcttgcctgtgtatacccgcaagactttcgagcgcgagctggctctggagtggatcattccagatgcagaggaagcataaaccggttttaaatatgtcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgaaacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaacttgatagtccggacctgcaggacgcgtgtcgatcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaa

SEQ ID NO: 26 - DNA, 6890 bp, synthetic construct
  source 1..6890: mol_type = other DNA; note = pET21 plasmid vector carrying the sequence for BSMV-CP fused with a linker and an OAS from a tobacco mosaic virus
ccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgatgcccaacgtatcactgacagcgaaagggggaggtcattacatcgaagatcagtgggatacgcaagtcgtcgaagcaggagtgttcgacgactggtgggtgcatgtagaggcctggaataagtttcttgacaatctgcgcggcatcaatttttccgtcgccagcagtcgctcacaagtagcagagtatttggctgctttggatcgtgaccttccggctgacgttgatcgtcgtttcgcgggtgcacgtggtcaaatcggcagccccaattacttaccagcacctaaattttttcgtcttgataaacgtacaatcgctgaattgacacgtttgtcgcgcttgacggatcagccccacaacaatcgtgatatcgagttaaatcgcgcaaaacgcgcaacaacaaatcctagcccacctgctcaagccccgagcgaaaaccttacactgcgcgacgtgcaacccttaaaggactccgcgttacattatcagtatgtccttattgatcttcagtccgcacgcttgcctgtgtatacccgcaagactttcgagcgcgagctggctctggagtggatcattccagatgcagaggaagcataaaccggttttaaatatgtcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgaaacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaacttgatagtccggacctgcaggacgcgtgtcgacgtttgagagagaagattacaaacgtgagagacggagggcccatggaacttacagaagaagtcgttgatgagttcatggaagatgtccctatgtcgatcaggcttgcaaagtttcgatctcgaaccggcctaggctcgtggtattgtttatagaaataatataaaattaggtttgagagagaagattacaaacgtgagagacggagggcccatggaacttacagaagaagtcgttgatgagttcatggaagatgtccctatgtcgatcaggcttgcaaagtttcgatctcgaaccggaaaaaagagtgatgtccgcaaagggaaaaatactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataat

SEQ ID NO: 27 - DNA, 31 bp, synthetic construct
  source 1..31: mol_type = other DNA; note = forward primer used to construct pET21-BSMV-CP-E37Q
tggtgggtgcatgtacaggcctggaataagt

SEQ ID NO: 28 - DNA, 31 bp, synthetic construct
  source 1..31: mol_type = other DNA; note = reverse primer used to construct pET21-BSMV-CP-E37Q
acttattccaggcctgtacatgcacccacca

SEQ ID NO: 29 - DNA, 33 bp, synthetic construct
  source 1..33: mol_type = other DNA; note = forward primer used to construct pET21-BSMV-CP-E37R
tggtgggtgcatgtacgtgcctggaataagttt

SEQ ID NO: 30 - DNA, 33 bp, synthetic construct
  source 1..33: mol_type = other DNA; note = reverse primer used to construct pET21-BSMV-CP-E37R
aaacttattccaggcacgtacatgcacccacca

SEQ ID NO: 31 - DNA, 31 bp, synthetic construct
  source 1..31: mol_type = other DNA; note = forward primer used to construct pET21-BSMV-CP-E62Q and pET21-BSMV-E62Q/D101N
cgctcacaagtagcacagtatttggctgctt

SEQ ID NO: 32 - DNA, 31 bp, synthetic construct
  source 1..31: mol_type = other DNA; note = reverse primer used to construct pET21-BSMV-CP-E62Q and pET21-BSMV-E62Q/D101N
aagcagccaaatactgtgctacttgtgagcg

SEQ ID NO: 33 - DNA, 31 bp, synthetic construct
  source 1..31: mol_type = other DNA; note = forward primer used to construct pET21-BSMV-CP-D68N
tatttggctgctttgaatcgtgaccttccgg

SEQ ID NO: 34 - DNA, 31 bp, synthetic construct
  source 1..31: mol_type = other DNA; note = reverse primer used to construct pET21-BSMV-CP-D68N
ccggaaggtcacgattcaaagcagccaaata

SEQ ID NO: 35 - DNA, 31 bp, synthetic construct
  source 1..31: mol_type = other DNA; note = forward primer used to construct pET21-BSMV-CP-D70N
gctgctttggatcgtaaccttccggctgacg

SEQ ID NO: 36 - DNA, 31 bp, synthetic construct
  source 1..31: mol_type = other DNA; note = reverse primer used to construct pET21-BSMV-CP-D70N
cgtcagccggaaggttacgatccaaagcagc

SEQ ID NO: 37 - DNA, 31 bp, synthetic construct
  source 1..31: mol_type = other DNA; note = forward primer used to construct pET21-BSMV-CP-D101N and pET21-BSMV-CP-E62Q/D101N
aaattttttcgtcttaataaacgtacaatcg

SEQ ID NO: 38 - DNA, 31 bp, synthetic construct
  source 1..31: mol_type = other DNA; note = reverse primer used to construct pET21-BSMV-CP-D101N and pET21-BSMV-CP-E62Q/D101N
cgattgtacgtttattaagacgaaaaaattt

SEQ ID NO: 39 - DNA, 33 bp, synthetic construct
  source 1..33: mol_type = other DNA; note = forward primer used to construct pET21-BSMV-CP-D101K
aaattttttcgtcttaaaaaacgtacaatcgct

SEQ ID NO: 40 - DNA, 33 bp, synthetic construct
  source 1..33: mol_type = other DNA; note = DNA reverse primer used to construct pET21-BSMV-CP-D101K
agcgattgtacgttttttaagacgaaaaaattt

SEQ ID NO: 41 - DNA, 32 bp, synthetic construct
  source 1..32: mol_type = other DNA; note = forward primer used to construct pET21-BSMV-CP-D101R
aaattttttcgtcttcgtaaacgtacaatcgc

SEQ ID NO: 42 - DNA, 32 bp, synthetic construct
  source 1..32: mol_type = other DNA; note = reverse primer used to construct pET21-BSMV-CP-D101R
gcgattgtacgtttacgaagacgaaaaaattt

SEQ ID NO: 43 - DNA, 20 bp, synthetic construct
  source 1..20: mol_type = other DNA; note = forward primer used to construct pET21-BSMV-CP-D199K
ggaattgtgagcggataaca

SEQ ID NO: 44 - DNA, 34 bp, synthetic construct
  variation 15: note = n is a, c, g, or t
  source 1..34: mol_type = other DNA; note = reverse primer used to construct pET21-BSMV-CP-D199K
  variation 16: note = n is a, c, g, or t
  misc_feature 5..10: note = introduced AgeI site
tattaccggtttamnntgcttcctctgcatctgg

(19) United States
(12) Patent Application Publication
(10) Pub. No.: US 2024/0209382 A1
(43) Pub. Date: Jun. 27, 2024

(54) NANOPARTICLES AND BIOTEMPLATES WITH TUNABLE LENGTH AND METHODS OF MANUFACTURING THE SAME

(71) Applicants: Purdue Research Foundation, West Lafayette, IN (US); University of Delaware, Newark, DE (US)

(72) Inventors: Kevin SOLOMON, Newark, DE (US); Kok Zhi LEE, Lafayette, IN (US); Akash VAIDYA, Newark, DE (US); Mruthula RAMMOHAN, Newark, DE (US)

(21) Appl. No.: 18/516,243

(22) Filed: Nov. 21, 2023

Related U.S. Application Data
(63) Continuation-in-part of application No. 16/805,305, filed on Feb. 28, 2020, now Pat. No. 11,820,988.
(60) Provisional application No. 62/811,756, filed on Feb. 28, 2019.

(51) Int. Cl. C12N 15/70; B82Y 5/00; C07K 14/005; C12N 7/00

(52) U.S. Cl. CPC C12N 15/70; C07K 14/005; C12N 7/00; B82Y 5/00; C12N 2770/00022; C12N 2770/00023

(57) ABSTRACT

Methods and nucleic acid sequences for the synthesis of biotemplates in a non-plant based expression system are provided. Such biotemplates include Barley stripe mosaic virus viral-like particles (BSMV-VLPs) that are capable of self-assembly due to being operatively linked with an origin of self-assembly with the Barley stripe mosaic virus capsid protein (BSMV-CP). Also provided are BSMV-VLPs that are capable of self-assembly due one or more site-directed mutations on the BSMV-CP, and BSMV-VLPs that exhibit enhanced stability due to such site-directed mutation(s).

### PRIORITY

This application is a related to, a continuation-in-part application of, and claims the priority benefit of U.S. patent application Ser. No. 16/805,305 filed Feb. 28, 2020, which issues as U.S. Pat. No. 11,820,988 on Nov. 21, 2023, which is related to and claims the priority benefit of U.S. Provisional Patent Application No. 62/811,756 to Solomon et al. filed Feb. 28, 2019. The entire contents of the aforementioned priority applications are hereby incorporated by reference in their entireties into this disclosure.

### BACKGROUND

A nanoparticle is a nano-object or particle between 1 and 100 nanometers (nm) in size. These structures are of scientific interest because of their beneficial physical, chemical, and electronic properties which lead to great potential for a broad range of applications in various fields, such as biomedicine, materials science, electronics, consumer products, cosmetics, pharmaceuticals, transportation, and energy. Of particular interest in the optoelectrical, electrical, biochemical, and medical fields are metal nanoparticles or metallized nanoparticles due to their unique properties.

The properties and functions of nanoparticles and nanostructures are often closely correlated with the size, shape, structure, surface area, and/or composition thereof. However, achieving the synthesis of nanoparticles having controllable dimensions and morphology, as well as high purity, quantity and quality remains a challenge. One type of bottom-up nanofabrication called biotemplating has proven to be viable means to easily and inexpensively produce nanomaterials on a large scale. More specifically, biotemplating is the use of naturally occurring biomolecules to develop nanomaterials of similar morphology, hierarchical complexity, and nanometric precision. Naturally occurring biomolecules, such as viruses, DNA, proteins, and RNA, offer highly ordered morphologies and well-defined architectures and organizations and, thus, provide the ability to generate uniform and monodisperse nanomaterials.

Viruses specifically are attractive scaffolds for the construction of hierarchical complex nanomaterials due to their unique advantages for applications in catalysis, nanocircuitry, chemical sensing, biocatalysis, memory devices, and light harvesting. Plant viruses, in particular, are small in size, display structural symmetry, ease of functionalization, and monodispersity, and spontaneously self-assemble into uniform nanoscale structures. Additionally, they often have wide range of stabilities to temperature, pH, salt, chemicals, and protease degradation. Plant viruses are relatively easy to purify as they lack membranes and have one or two protein capsid assemblies that are structurally defined. In addition to allowing for the production of novel nanomaterials in a very precise and controlled fashion, the ability to genetically and chemically modify plant viruses also allows for the insertion or replacement of selected amino acids on virus capsids for uses ranging from bioconjugation to mineralization.

The M13 bacteriophage, the Tobacco mosaic virus (TMV), and their engineered variants remain the prevalent biotemplates employed in conventional nanowire and nanorod synthesis. TMV, in particular, has been exploited as a biotemplate in various applications, including battery electrodes, memory devices, catalysts, and chemical sensors, which are coated with metals such as silver, platinum, aluminum, palladium, gold and gold/palladium alloy. The Barley stripe mosaic virus (BSMV) has also been utilized for production of organic-metal nanorods, but to a lesser degree and such methods are limited to in-planta production, which limits its development.

Conventional nanoparticle synthesis platforms face certain drawbacks, especially with respect to viral systems produced in-planta. In addition to limiting development, the genomes of in-planta-synthesized viruses are subject to evolutionary pressures that may remove engineered modifications designed to enhance biotemplating functionality if such removal benefits viral fitness. As these viruses are plant pathogens, virus-producing plants must also be grown in specialized facilities to control and/or prevent the spread of the pathogen to wild plants. Furthermore, the viral replication cycle in plants requires between 2-3 weeks, which results in a long and complicated process to extract a relatively small quantity of viruses. Finally, the conventional production of metal nanoparticles in particular requires varying conditions (e.g., pHs, temperatures, etc.) that may destabilize the produced nanoparticles. Accordingly, there is a need for an alternative approach that is inexpensive, highly precise, robust, efficient, and ideally tunable for particular applications.

### SUMMARY

Novel methods for manufacturing nanoparticle biotemplates are provided. In at least one embodiment, such methods comprise introducing into a host a nucleic acid sequence encoding a Barley stripe mosaic virus coat protein (BSMV-CP) comprising one or both of: (a) an origin of self-assembly (OAS) derived from a virus operatively linked with the BSMV-CP, and (b) at least one site-directed mutation on the BSMV-CP to strengthen an interaction between at least two BSMV-CP subunits. The nucleic acid sequence is then expressed in an expression system (microbial-based or otherwise) to allow expression of the BSMV-CP, which produces self-assembled BSMV viral-like particles (BSMV VLPs). The method further comprises isolating the BSMV VLPs from the expression system.

In at least one embodiment, the OAS is derived from Tobacco mosaic virus. Additionally, the OAS may comprise SEQ ID NO: 11 or a functional equivalent thereof.

Additionally or alternatively, the step of expressing the nucleic acid sequence may comprise: constructing a plasmid or an expression vector comprising the nucleic acid sequence and transforming the plasmid or expression vector into the host. In certain embodiments, the host may be Escherichia col. Furthermore, the step of expressing the nucleic acid sequence may be performed at a neutral pH. In at least one additional embodiment, the BSMV-CP may be fused with a linker region and comprise at least one site-directed mutation on the BSMV-CP to strengthen an interaction between at least two BSMV-CP subunits.

In an exemplary embodiment, the BSMV-CP further comprises a fusion of BSMV-CP, a linker region, and the OAS. There, the method may further comprise selecting a length of the linker region based on a desired length in the resulting BSMV VLPs such that the VLPs themselves are tunable and/or customizable.

Methods of the present disclosure may further comprise the step of synthesizing one or more nanoparticles using the resulting VLPs. Optionally, the method may comprise coating at least a surface of the resulting VLPs with a metal, where desired. Such coating may be performed using adsorption, for example, or via any other modality now known in the art or hereinafter developed. Still further, the methods hereof may comprise the step of performing microbial reduction of the resulting VLPs.

As previously noted, embodiments of the presently disclosed methods may comprise BSMV-CP comprising at least one site-directed mutation on the BSMV-CP to strengthen an interaction between at least two BSMV-CP subunits. There, for example and without limitation, the at least one site-directed mutation may be at one or more of the following residues: E37Q, E37R, E62Q, D68N, D70N, D101N, D101R, and D101K.

Novel nanoparticles are also described herein, as well as methods for manufacturing the same. In at least one exemplary embodiment, the present disclosure provides a nanoparticle manufactured according to a process comprising the steps of: introducing into a host a nucleic acid sequence encoding a Barley stripe mosaic virus coat protein (BSMV-CP) comprising one or both of: (a) an origin of self-assembly (OAS) derived from a virus operatively linked with the BSMV-CP, and (b) at least one site-directed mutation on the BSMV-CP to strengthen an interaction between at least two BSMV-CP subunits; expressing the nucleic acid sequence in an expression system to allow expression of the BSMV-CP and produce self-assembled BSMV viral-like particles (BSMV VLPs); isolating the BSMV VLPs from the expression system; and synthesizing one or more nanoparticles using the BSMV VLPs as a biotemplate. There, the nucleic acid sequence may be modified such that it further encodes a linker region comprising a length that is fused with at least the BSMV-CP. As the length of the linker region will directly correlate the size shapes of the VLPs produced through the method, the length of the linker region may be specifically selected to customize the dimensions of the resulting nanoparticles. In other words, the length of the linker region may directly correlate with the length of the resulting nanoparticles by function of influencing the size and/or shape of the VLPs biotemplates.

Additionally, where the BSMV-CP comprises the at least one site-directed mutation, such site-directed mutation may be at one or more of the following residues thereof: E37Q, E37R, E62Q, D68N, D70N, and D101N.

In at least one exemplary embodiment of the nanoparticles provided herein, the step of introducing into a host a nucleic acid sequence may further comprise: constructing a plasmid or expression vector comprising the nucleic acid sequence and transforming the plasmid or expression vector into the host (for example, and without limitation, Escherichia coli). Optionally, the OAS comprises SEQ ID NO: 11 or a functional equivalent thereof. The expression system may comprise any non-plant based expression system including a microbial-based, insect-based, or mammalian-based expression system.

Novel nucleic acid sequences are also provided herein. In at least one embodiment, a nucleic acid for synthesis of a nanoparticle biotemplate is described, such nucleic acid comprising all or part of a sequence encoding a Barley stripe mosaic virus coat protein (BSMV-CP) operatively linked to a linker region having a length and an origin of self-assembly (OAS) derived from a virus. In at least one exemplary embodiment, the BSMV-CP may comprise a protein sequence selected from a group consisting of: SEQ ID NO: 2, SEQ ID NO: 3; SEQ ID NO: 4, SEQ ID NO: 5, SEQ ID NO: 6, SEQ ID NO: 7, SEQ ID NO: 8, SEQ ID NO: 9, SEQ ID NO: 10, or comprise a functional equivalent of one of SEQ ID NOS: 2-10. Additionally or alternatively, a portion of the sequence for encoding the OAS may comprise SEQ ID NO: 11 or a functional equivalent thereof. In at least one exemplary embodiment, the sequence comprises SEQ ID NO: 15 or a functional equivalent thereof.

VLPs are also provided. In certain embodiments, a VLP is provided that comprises one or both of: (a) an OAS operatively linked with a BSMV-CP, wherein a portion of the nucleic acid sequence that encodes the OAS comprises SEQ ID NO: 11 or a functional equivalent thereof, and (b) at least one site-directed mutation on the BSMV-CP at residue E62, D101, or both residues E62 and D101, wherein each site-directed mutation independently comprises a neutral or positive amino acid; and wherein the VLP is stable at a pH of at or between about 4 to about 9.

The site-directed mutation at residue E62, D101, or both residues E62 and D101 on the BSMV-CP can each independently comprise glutamine, asparagine, arginine, or lysine. The site-directed mutation at residue E62 can comprise SEQ ID NO: 9 or a functional variant thereof. The site-directed mutation at residue D101 can comprise SEQ ID NO: 5 or SEQ ID NO: 6, or a functional variant of SEQ ID NO: 5 or SEQ ID NO: 6. The site-directed mutation at both residues E62 and D101 can comprise SEQ ID NO: 10 or a functional variant thereof.

The VLP can comprise a rod length of at or between about 75 nm to about 150 nm.

The VLP can comprise surface-exposed C-termini. In certain embodiments, the surface-exposed C-termini comprise a site-directed insertion of a natural amino acid residue or a SpyTag. In certain embodiments, the natural amino acid residue comprises a lysine, a histidine, or a cysteine residue.

The surface exposed C-termini can be functionalized with a ligand. The ligand can, in certain embodiments, comprise a fluorescent label, an amide, a reactive electrophile, a peptide, a polymer, a small molecule, a metal, or a protein. The fluorescent label can comprise fluorescamine or fluorescein malemide, for example.

Methods of manufacturing a nanoparticle biotemplate are also provided. In certain embodiments, such methods comprise the steps of: introducing into an isolated host a nucleic acid sequence that encodes a BSMV-CP and one or both of: (a) an OAS operatively linked with the BSMV-CP wherein a portion of the nucleic acid sequence that encodes the OAS comprises SEQ ID NO: 11 or a functional equivalent thereof, and (b) at least one site-directed mutation on the BSMV-CP at residue E62, D101, or both residues E62 and D101, wherein each site-directed mutation independently comprises a neutral or positive amino acid; expressing the nucleic acid sequence in a microbial expression system to produce self-assembled BSMV VLPs; and isolating the BSMV VLPs from the microbial expression system.

The nucleic acid sequence can encode any of the VLPs described herein. In certain embodiments, the nucleic acid sequence comprises a site-directed mutation at residue E62, D101, or both residues E62 and D101 on the BSMV-CP. There, the site-directed mutation at residue E62, D101, or both residues E62 and D101 on the BSMV-CP can be independently selected from the group consisting of glutamine, asparagine, arginine, and lysine.

The resulting BSMV VLPs can be stable at a pH of at or between 4-9. The resulting BSMV VLPs can be stable at a pH of about 4. The resulting BSMV VLPs can be stable at a pH of about 9. The resulting BSMV VLPs can comprise a higher average rod length as compared to wildtype BSMV VLPs at a neutral pH. The step of expressing the nucleic acid sequence can be performed at a pH of about 4 or about 9.

The nucleic acid sequence can further comprise a site-directed mutation at a C-terminus of the BSMV-CP that encodes a natural amino acid residue or a SpyTag for display on a surface of the resulting BSMV VLP. The natural amino acid residue can comprise a lysine, a histidine, or a cysteine.

In certain embodiments, the method further comprises functionalizing the surface of the BSMV VLP with one or more ligands. In certain embodiments of the method, the step of expressing the nucleic acid sequence further comprises: constructing a plasmid or an expression vector comprising the nucleic acid sequence; and transforming the plasmid or expression vector into the host; wherein the host is E. coli and the step of expressing the nucleic acid sequence is performed at a pH at or between 4-9.

The BSMV-CP can comprise a BSMV-CP hereof fused with a linker region. In certain embodiments, the BSMV-CP comprises at least one site-directed mutation at residue E62, D101, or both residues E62 and D101 on the BSMV-CP.

In certain embodiments, the method can further comprise the step of selecting a length of the linker region based on a desired length in the resulting BSMV VLPs. Additionally or alternatively, the method can comprise the step of synthesizing one or more nanoparticles using the resulting VLPs.

### BRIEF DESCRIPTION OF THE SEQUENCE LISTINGS

SEQ ID NO: 1 is an amino acid sequence of a wild-type Barley stripe mosaic virus coat protein (BSMV-CP):

N-MPNVSLTAKGGGHYIEDQWDTQVVEAGVFDDWWVHVEAWNKFLDNLRG
INFSVASSRSQVAEYLAALDRDLPADVDRRFAGARGQIGSPNYLPAPKFF
RLDKRTIAELTRLSRLTDQPHNNRDIELNRAKRATTNPSPPAQAPSENLT
LRDVQPLKDSALHYQYVLIDLQSARLPVYTRKTFERELALEWIIPDAE
EA-C;

SEQ ID NO: 2 is an artificial amino acid sequence of at least one exemplary embodiment of a BSMV-CP having a point mutation at residue D68N according to the present disclosure:

N-MPNVSLTAKGGGHYIEDQWDTQVVEAGVFDDWWVHVEAWNKFLDNLRG
INFSVASSRSQVAEYLAALNRDLPADVDRRFAGARGQIGSPNYLPAPKFF
RLDKRTIAELTRLSRLTDQPHNNRDIELNRAKRATTNPSPPAQAPSENLT
LRDVQPLKDSALHYQYVLIDLQSARLPVYTRKTFERELALEWIIPDAE
EA-C;

SEQ ID NO: 3 is an artificial amino acid sequence of at least one exemplary embodiment of a BSMV-CP having a point mutation at residue D70N according to the present disclosure:

N-MPNVSLTAKGGGHYIEDQWDTQVVEAGVFDDWWVHVEAWNKFLDNLRG
INFSVASSRSQVAEYLAALDRNLPADVDRRFAGARGQIGSPNYLPAPKFF
RLDKRTIAELTRLSRLTDQPHNNRDIELNRAKRATTNPSPPAQAPSENLT
LRDVQPLKDSALHYQYVLIDLQSARLPVYTRKTFERELALEWIIPDAE
EA-C;

SEQ ID NO: 4 is an artificial amino acid sequence of at least one exemplary embodiment of a BSMV-CP having a point mutation at residue D101K according to the present disclosure:

N-MPNVSLTAKGGGHYIEDQWDTQVVEAGVFDDWWVHVEAWNKFLDNLRG
INFSVASSRSQVAEYLAALDRDLPADVDRRFAGARGQIGSPNYLPAPKFF
RLKKRTIAELTRLSRLTDQPHNNRDIELNRAKRATTNPSPPAQAPSENLT
LRDVQPLKDSALHYQYVLIDLQSARLPVYTRKTFERELALEWIIPDAE
EA-C;

SEQ ID NO: 5 is an artificial amino acid sequence of at least one exemplary embodiment of a BSMV-CP having a point mutation at residue D101N according to the present disclosure, such mutant being verified as capable of self-assembly:

N-MPNVSLTAKGGGHYIEDQWDTQVVEAGVFDDWWVHVEAWNKFLDNLRG
INFSVASSRSQVAEYLAALDRDLPADVDRRFAGARGQIGSPNYLPAPKFF
RLNKRTIAELTRLSRLTDQPHNNRDIELNRAKRATTNPSPPAQAPSENLT
LRDVQPLKDSALHYQYVLIDLQSARLPVYTRKTFERELALEWIIPDAE
EA-C;

SEQ ID NO: 6 is an artificial amino acid sequence of at least one exemplary embodiment of a BSMV-CP having a point mutation at residue D101R according to the present disclosure:

N-MPNVSLTAKGGGHYIEDQWDTQVVEAGVFDDWWVHVEAWNKFLDNLRG
INFSVASSRSQVAEYLAALDRDLPADVDRRFAGARGQIGSPNYLPAPKFF
RLRKRTIAELTRLSRLTDQPHNNRDIELNRAKRATTNPSPPAQAPSENLT
LRDVQPLKDSALHYQYVLIDLQSARLPVYTRKTFERELALEWIIPDAE
EA-C;

SEQ ID NO: 7 is an artificial amino acid sequence of at least one exemplary embodiment of a BSMV-CP having a point mutation at residue E37Q according to the present disclosure:

N-MPNVSLTAKGGGHYIEDQWDTQVVEAGVFDDWWVHVQAWNKFLDNLRG
INFSVASSRSQVAEYLAALDRDLPADVDRRFAGARGQIGSPNYLPAPKFF
RLDKRTIAELTRLSRLTDQPHNNRDIELNRAKRATTNPSPPAQAPSENLT
LRDVQPLKDSALHYQYVLIDLQSARLPVYTRKTFERELALEWIIPDAE
EA-C;

SEQ ID NO: 8 is an artificial amino acid sequence of at least one exemplary embodiment of a BSMV-CP having a point mutation at residue E37R according to the present disclosure:

N-MPNVSLTAKGGGHYIEDQWDTQVVEAGVFDDWWVHVRAWNKFLDNLRG
INFSVASSRSQVAEYLAALDRDLPADVDRRFAGARGQIGSPNYLPAPKFF
RLDKRTIAELTRLSRLTDQPHNNRDIELNRAKRATTNPSPPAQAPSENLT
LRDVQPLKDSALHYQYVLIDLQSARLPVYTRKTFERELALEWIIPDAEE
A-C;

SEQ ID NO: 9 is an artificial amino acid sequence of at least one exemplary embodiment of a BSMV-CP having a point mutation at residue E62Q according to the present disclosure, such mutant being verified as capable of self-assembly:

N-MPNVSLTAKGGGHYIEDQWDTQVVEAGVFDDWWVHVEAWNKFLDNLRG
INFSVASSRSQVAQYLAALDRDLPADVDRRFAGARGQIGSPNYLPAPKFF
RLDKRTIAELTRLSRLTDQPHNNRDIELNRAKRATTNPSPPAQAPSENLT
LRDVQPLKDSALHYQYVLIDLQSARLPVYTRKTFERELALEWIIPDAE
EA-C;

SEQ ID NO: 10 is an artificial amino acid sequence of at least one exemplary embodiment of a BSMV-CP having point mutations at both residues E62Q and D101N according to the present disclosure:

N-MPNVSLTAKGGGHYIEDQWDTQVVEAGVFDDWWVHVEAWNKFLDNLRG
INFSVASSRSQVAQYLAALDRDLPADVDRRFAGARGQIGSPNYLPAPKFF
RLNKRTIAELTRLSRLTDQPHNNRDIELNRAKRATTNPSPPAQAPSENLT
LRDVQPLKDSALHYQYVLIDLQSARLPVYTRKTFERELALEWIIPDAE
EA-C;

SEQ ID NO: 11 is a DNA sequence that encodes a wild-type origin of self-assembly derived from a Tobacco mosaic virus:

5′-GTATTGTTTATAGAAATAATATAAAATTAGGTTTGAGAGAGAAGATT
ACAAACGTGAGAGACGGAGGGCCCATGGAACTTACAGAAGAAGTCGTTGA
TGAGTTCATGGAAGATGTCCCTATGTCGATCAGGCTTGCAAAGTTTCGAT
CTCGAACCGGAAAAAAGAGTGATGTCCGCAAAGGGAAAAATA-3′;

SEQ ID NO: 12 is a DNA sequence that encodes a wild-type BSMV-CP:

5′-ATGCCCAACGTATCACTGACAGCGAAAGGGGGAGGTCATTACATCGA
AGATCAGTGGGATACGCAAGTCGTCGAAGCAGGAGTGTTCGACGACTGGT
GGGTGCATGTAGAGGCCTGGAATAAGTTTCTTGACAATCTGCGCGGCATC
AATTTTTCCGTCGCCAGCAGTCGCTCACAAGTAGCAGAGTATTTGGCTGC
TTTGGATCGTGACCTTCCGGCTGACGTTGATCGTCGTTTCGCGGGTGCAC
GTGGTCAAATCGGCAGCCCCAATTACTTACCAGCACCTAAATTTTTTCGT
CTTGATAAACGTACAATCGCTGAATTGACACGTTTGTCGCGCTTGACGGA
TCAGCCCCACAACAATCGTGATATCGAGTTAAATCGCGCAAAACGCGCAA
CAACAAATCCTAGCCCACCTGCTCAAGCCCCGAGCGAAAACCTTACACTG
CGCGACGTGCAACCCTTAAAGGACTCCGCGTTACATTATCAGTATGTCCT
TATTGATCTTCAGTCCGCACGCTTGCCTGTGTATACCCGCAAGACTTTCG
AGCGCGAGCTGGCTCTGGAGTGGATCATTCCAGATGCAGAGGAAGCATA
A-3′;

SEQ ID NO: 13 is an artificial sequence of at least one exemplary embodiment that encodes a linker region according to the present disclosure;

SEQ ID NO: 14 is an artificial DNA sequence of at least one exemplary embodiment that encodes a BSMV-CP (SEQ ID NO: 12) fused with an OAS derived from TMV (SEQ ID NO: 11);

SEQ ID NO: 15 is an artificial DNA sequence of at least one exemplary embodiment that encodes a BSMV-CP (SEQ ID NO: 12) fused with a linker region and an OAS derived from TMV (SEQ ID NO: 11);

SEQ ID NO: 16 is an artificial DNA sequence of a plasmid vector pET21-BSMV-D70N that encodes the protein of SEQ ID NO: 3;

SEQ ID NO: 17 is an artificial DNA sequence of a plasmid vector pET21-BSMV-D68N that encodes the protein of SEQ ID NO: 2;

SEQ ID NO: 18 is an artificial DNA sequence of a plasmid vector pET21-BSMV-D101K that encodes the protein of SEQ ID NO: 4;

SEQ ID NO: 19 is an artificial DNA sequence of a plasmid vector pET21-BSMV-D101R that encodes the protein of SEQ ID NO: 6;

SEQ ID NO: 20 is an artificial DNA sequence of a plasmid vector pET21-BSMV-E37Q that encodes the protein of SEQ ID NO: 7;

SEQ ID NO: 21 is an artificial DNA sequence of a plasmid vector pET21-BSMV-E37R that encodes the protein of SEQ ID NO: 8;

SEQ ID NO: 22 is an artificial DNA sequence of a plasmid vector pET21-BSMV-D101N that encodes the protein of SEQ ID NO: 5;

SEQ ID NO: 23 is an artificial DNA sequence of a plasmid vector pET21-BSMV-E62Q that encodes the protein of SEQ ID NO: 9;

SEQ ID NO: 24 is an artificial DNA sequence of a plasmid vector pET21-BSMV-E62Q/D101N that encodes the protein of SEQ ID NO: 10;

SEQ ID NO: 25 is an artificial DNA sequence of a plasmid vector pET21-BSMV;

SEQ ID NO: 26 is an artificial DNA sequence of a plasmid vector pET21-BSMV-Linker-OAS that encodes a BSMV-CP (SEQ ID NO: 12) fused with a linker region and an OAS derived from TMV (SEQ ID NO: 11);

SEQ ID NO: 27 is an artificial DNA forward primer used to construct pET21-BSMV-CP-E37Q, with modified nucleotides corresponding to the modified codons in lowercase text:

TGGTGGGTGCATGTAcAGGCCTGGAATAAGT;

SEQ ID NO: 28 is an artificial DNA reverse primer used to construct pET21-BSMV-CP-E37Q, with modified nucleotides corresponding to the modified codons in lowercase text:

ACTTATTCCAGGCCTgTACATGCACCCACCA;

SEQ ID NO: 29 is an artificial DNA forward primer used to construct pET21-BSMV-CP-E37R, with modified nucleotides corresponding to the modified codons in lowercase text:

TGGTGGGTGCATGTAcgtGCCTGGAATAAGTTT;

SEQ ID NO: 30 is an artificial DNA reverse primer used to construct pET21-BSMV-CP-E37R, with modified nucleotides corresponding to the modified codons in lowercase text:

AAACTTATTCCAGGCacgTACATGCACCCACCA;

SEQ ID NO: 31 is an artificial DNA forward primer used to construct pET21-BSMV-CP-E62Q and pET21-BSMV-E62Q/D101N, with modified nucleotides corresponding to the modified codons in lowercase text:

CGCTCACAAGTAGCAcAGTATTTGGCTGCTT;

SEQ ID NO: 32 is an artificial DNA reverse primer used to construct pET21-BSMV-CP-E62Q and pET21-BSMV-E62Q/D101N, with modified nucleotides corresponding to the modified codons in lowercase text:

AAGCAGCCAAATACTgTGCTACTTGTGAGCG;

SEQ ID NO: 33 is an artificial DNA forward primer used to construct pET21-BSMV-CP-D68N, with modified nucleotides corresponding to the modified codons in lowercase text:

TATTTGGCTGCTTTGaATCGTGACCTTCCGG;

SEQ ID NO: 34 is an artificial DNA reverse primer used to construct pET21-BSMV-CP-D68N, with modified nucleotides corresponding to the modified codons in lowercase text:

CCGGAAGGTCACGATtCAAAGCAGCCAAATA;

SEQ ID NO: 35 is an artificial DNA forward primer used to construct pET21-BSMV-CP-D70N, with modified nucleotides corresponding to the modified codons in lowercase text:

GCTGCTTTGGATCGTaACCTTCCGGCTGACG;

SEQ ID NO: 36 is an artificial DNA reverse primer used to construct pET21-BSMV-CP-D70N, with modified nucleotides corresponding to the modified codons in lowercase text:

CGTCAGCCGGAAGGTtACGATCCAAAGCAGC;

SEQ ID NO: 37 is an artificial DNA forward primer used to construct pET21-BSMV-CP-D101N and pET21-BSMV-CP-E62Q/D101N, with modified nucleotides corresponding to the modified codons in lowercase text:

AAATTTTTTCGTCTTaATAAACGTACAATCG;

SEQ ID NO: 38 is an artificial DNA reverse primer used to construct pET21-BSMV-CP-D101N and pET21-BSMV-CP-E62Q/D101N, with modified nucleotides corresponding to the modified codons in lowercase text:

CGATTGTACGTTTATtAAGACGAAAAAATTT;

SEQ ID NO: 39 is an artificial DNA forward primer used to construct pET21-BSMV-CP-D101K, with modified nucleotides corresponding to the modified codons in lowercase text:

AAATTTTTTCGTCTTaaaAAACGTACAATCGCT;

SEQ ID NO: 40 is an artificial DNA reverse primer used to construct pET21-BSMV-CP-D101K, with modified nucleotides corresponding to the modified codons in lowercase text:

AGCGATTGTACGTTTtttAAGACGAAAAAATTT;

SEQ ID NO: 41 is an artificial DNA forward primer used to construct pET21-BSMV-CP-D101R, with modified nucleotides corresponding to the modified codons in lowercase text:

AAATTTTTTCGTCTTcgTAAACGTACAATCGC;

SEQ ID NO: 42 is an artificial DNA reverse primer used to construct pET21-BSMV-CP-D101R, with modified nucleotides corresponding to the modified codons in lowercase text:

GCGATTGTACGTTTAcgAAGACGAAAAAATTT;

SEQ ID NO: 43 is an artificial DNA forward primer used to construct pET21-BSMV-CP-D199K:

GGAATTGTGAGCGGATAACA;

and

SEQ ID NO: 44 is an artificial DNA reverse primer used to construct pET21-BSMV-CP-D199K, with modified nucleotides corresponding to the modified codons in lowercase text and introduced AgeI sites in underlined text: TATTACCGGTTTAmnnTGCTTCCTCTGCATCTGG.

In addition to the foregoing, written Sequence Listings for the above-described sequences are appended hereto and the same Sequence Listings are provided in computer readable form in a Sequence Listing XML file (file entitled “68559-03SequenceListingST.26_8MAR2024”; file size=131,856 bytes; date created Mar. 8, 2024) filed herewith and herein incorporated by reference. The information recorded in computer readable form is identical to the written Sequence Listing provided herein, pursuant to 37 C.F.R. § 1.821(f).

### BRIEF DESCRIPTION OF THE DRAWINGS

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings, wherein:

FIG. 1 shows various views of a graphical model of the structure of the Barley stripe mosaic virus (BSMV) (PDB:5A7A), with subpart (a) showing a perspective view of a BSMV capsid protein; subpart (b) showing a side view of an assembled BSMV virion; and subpart (c) showing a top-down view of assembled BSMV capsid proteins (images adapted from Clare et al., Novel Inter-Subunit Contacts in Barley Stripe Mosaic Virus Revealed by Cryo-Electron Microscopy, Structure, Oct. 6, 2015; 23(10): 1815-1826);

FIGS. 2A and 2B show representations of an exemplary method for producing BSMV-VLPs in a bacterial expression system according to at least one embodiment of the present disclosure, with FIG. 2A showing a schematic of the method and FIG. 2B showing a flow-chart representative of the method;

FIG. 3 shows a schematic interpretation of a TMV coat protein and origin of assembly (OAS) on TMV RNA, with subpart (a) showing a completed self-assembly into a helical structure; subpart (b) showing a partial nucleotide sequence of SEQ ID No. 11 and secondary structure of the OAS on TMV RNA; and subpart (c) showing a schematic interpretation of the structures of a TMV coat protein and OAS and its steps of self-assembly.

FIG. 4 represents BSMV-viral capsid protein (BSMV-CP) constructs that include a linker RNA, with subpart (A) showing a BSMV-CP fused to an artificial linker with 1322 base transcript length and no OAS (top) and a BSMV-CP fused with an artificial linker and an OAS sequence from TMV, with 1652 base transcript length, subpart (B) showing a visualization of a BSMV-CP-linker by transmission electronic microscope (TEM), and subpart (C) showing a visualization of BSMV-CP-linker-OAS and resulting self-assembly by TEM;

FIG. 5, subpart (a) shows a schematic representation of tunable BSMV-VLPs—BSMV coat protein fused to a customizable length of linker and an OAS;

FIG. 5, subpart (b) shows a schematic representation that engineered BSMV-VLPs with increased coat protein interaction are less susceptible to extreme pH and low calcium ion concentrations;

FIG. 5, subpart (c) shows a schematic representation of all BSMV protein constructs expressed and produced in a microbial-based chassis;

FIG. 6 illustrates various graphical models of interacting residues between BSMV coat protein subunits, with subpart (a) showing that the interacting residues of TMV (PDB:2xea) are D77 and E50 residues of the coat protein subunit; subpart (b) showing likely interacting residues of BSMV (PDB:5a7a) corresponding to that of TMV, which includes E62, D68, D70, and D101 based on the present investigators' data; and subpart (c) showing predicted native interacting residues of BSMV in the literature, which includes E37, D70, and D74;

FIG. 7 shows TEM visualization of BSMV-CP-linker carrying a point mutation at E37R (subpart A), a point mutation at E37Q (subpart B), a point mutation D101N (subpart C), a point mutation at E62Q (subpart D), point mutations at both E62Q and D101N (subpart E), a point mutation at D101R (subpart F), and a point mutation at D101K (subpart G), with the self-assembly of rod-shaped VLPs being verified at least in the D101N, E62Q, E62Q/D101N, D101R, and D101K point mutations;

FIG. 8 shows PAGE analysis results of a BSMV-CP purification scheme, with Lane 1 indicative of whole cell lysate before IPTG induction; Lane 2 indicative of whole cell lysate after IPTG induction; Lane 3 indicative of whole cell lysate after Bugbuster; Lane 4 indicative of insoluble bacterial pellet; Lane 5 indicative of supernatant after 4.5/5 min before 50 k/30′; Lane 6 indicative of insoluble pellet after 50 k/30 min; Lane 7 indicative of pellet after insoluble pellet removed and 3 k/5 min; and Lane 8 indicative of final resuspension; BSMV-CP is ˜22.5 kDa;

FIG. 9 shows PAGE analysis results of BSMV-CP expression conducted at 23° C. and 37° C., respectively, with P representing pellet and S representing supernatant after 92,000×g and 20 min centrifuge and arrow mark indicating BSMV-CP at ˜22.5 kDa;

FIG. 10 shows PAGE analysis results of BSMV-CP expression conducted at different inducer agent concentrations, with subpart (a) representing soluble fraction yield at 0.01 mM, 0.05 mM, 0.075 mM, and 0.10 mM, and subpart (b) representing the effect of final pellet resuspension buffers on solubilizing BSMV-CP in water (A), Tris buffer (B), and sodium phosphate buffer (C); arrow marks indicating BSMV-CP protein at ˜22.5 kDa;

FIG. 11 are TEM visualizations of BSMV-OAS after 68% sucrose cushion, with subpart (a) showing the top layer supernatant and subpart (b) showing the bottom of the 68% sucrose cushion;

FIG. 12 is a length distribution histogram of purified BSMV-VLPs produced according to at least one embodiment of the present disclosure; and

FIG. 13 is a TEM image of a palladium coated BMSV-VLP nanorod prepared according to at least one embodiment of the present disclosure.

FIGS. 14A-14B show mutating Caspar carboxylate cluster (CCC) residues predicted by cryo-EM to prevent VLP assembly, with FIG. 14A showing a schematic representation of E37, D70 and D74 in BSMV, and FIGS. 14B and 14C showing transmission electron microscope images of mutants E37Q (FIG. 14B) and E37R (FIG. 14C).

FIG. 14D shows SDS-PAGE analysis of BSMV-CP mutants and a negative control, with the arrowhead indicating the BSMV capsid protein at ˜22.5 kDa. These data support BSMV mutants were heterologously expressed in E. coli.

FIGS. 15A-15C show CCC carboxylates, with FIG. 15A showing a CCC of TMV (PDB:2xea), FIG. 15B showing potential CCC candidate residues (E62, D68, D70, and D101) in BSMV (PDB:5a7a) based on structural alignment studies, and FIG. 15C showing the overlay of FIGS. 15A and 15B. The predicted CCC was based on crystal structure literature.

FIG. 15D shows TEM images of certain mutations at CCC residues predicted by TMV overlay that prevented assembly, with subpart (a) showing D70N and subpart (b) showing D68N. Scale bar=50 nm.

FIGS. 16A-16B show transmission electron microscope (TEM) images of mutated CCC residues predicted by structural overlay to allow for VLP assembly, namely BSMV-CP single mutants E62Q (FIG. 16A) and D101N (FIG. 16B), and double mutant E62Q/D101N (FIG. 16C).

FIGS. 17A-17C show data relating to the D101R mutant VLPs stabilized in neutral conditions. FIG. 17A is a TEM image of wildtype BSMV VLPs, FIG. 17B is a TEM image of mutant BSMV-D101R VLPs, and FIG. 17C is a graph showing length distribution data at pH 7 of the mutant BSMV-D101R VLPs of FIG. 17B.

FIG. 17D shows a TEM image of the D101K mutant. Scale bar=50 nm.

FIGS. 18A-18C show data relating to D101R mutant VLPs stabilized in alkaline conditions. FIG. 18A is a TEM image of wildtype BSMV VLPs, FIG. 18B is a TEM image of mutant BSMV-D101R VLPs, and FIG. 18C is a graph showing length distribution data at pH 9 of the mutant BSMV-D101R VLPs of FIG. 18B.

FIGS. 19A and 19B show dynamic light scattering data relating to D101R mutants having a reduced fraction of disassembled rods, with FIG. 19A showing D101R mutant VLPs and FIG. 19B showing wildtype BSMV VLPs.

FIGS. 20A and 20B show D101R mutant VLPs assembled in acidic conditions, with FIG. 20A showing a TEM image of wildtype BSMV VLPs and FIG. 20B showing a TEM image of mutant BSMV-D101R VLPs, both at pH 4.

FIGS. 20C and 20D show TEM images of wildtype BSMV-VLPs after resuspension in pH 4 buffer. The wildtype BSMV-VLPs remain as rods at pH 4.

FIGS. 20E and 20F show TEM images of D101R mutant BSMV-VLPs after resuspension in pH 4 buffer. The D101R mutants remain as rods at pH 4.

FIGS. 21A, 21B, and 21C show data related to the surface display of lysine residue on D1010R mutant VLPs. FIG. 21A is a cryo-electron microscope elucidated structure of BSMV virion with surface exposed C-termini, FIG. 21B is an AlphaFold2 prediction of D101R dimer, and FIG. 21C is a TEM image of BSMV-D1010R/199K VLPs.

FIG. 21D shows SDS-PAGE data validating yield and purity of D101R/199K mutant VLPs.

FIGS. 22A and 22B related to the reaction between solvent-exposed primary amines on D101R 199K mutants and fluorescamine (supporting that this reaction occurs faster at neutral and alkaline conditions than acidic conditions). FIG. 22A is a schematic of the reaction between primary amine and fluorescamine. FIG. 22B is a graph showing the fluorescence intensity of fluorophore-conjugated primary amines at pH 5, 7, and 9 (error bars represent standard error of measurements (SEM) performed in triplicate).

FIG. 23A is a size exclusion chromatogram (SEC) profile of D101R/199C mutants incubated with and without dye.

FIG. 23B shows TEM images of both D101R/199C (top left) and D101R/199K (bottom left) mutants, and a graph of the measured affect of single-residue insertions on the resulting VLP surface charges.

FIG. 24 is a schematic illustrating the mechanism of the BSMV VLPs hereof functionalized with a SpyTag peptide.

FIG. 25 shows a TEM image of a BSMV VLP rod functionalized with a SpyTag peptide via direct fusion pursuant to the mechanism of FIG. 24.

While the present disclosure is susceptible to various modifications and alternative forms, exemplary embodiments thereof are shown by way of example in the drawings and are herein described in detail.

### DETAILED DESCRIPTION

For the purposes of promoting an understanding of the principles of the present disclosure, reference will now be made to the embodiments illustrated in the drawings and specific language will be used to describe the same. It will nevertheless be understood that no limitation of scope is intended by the description of these embodiments. On the contrary, this disclosure is intended to cover alternatives, modifications, and equivalents as may be included within the spirit and scope of this application as defined by the appended claims. As previously noted, while this technology may be illustrated and described in one or more preferred embodiments, the compositions, systems and methods hereof may comprise many different configurations, forms, materials, and accessories.

In the following description, numerous specific details are set forth in order to provide a thorough understanding of the present disclosure. Particular examples may be implemented without some or all of these specific details and it is to be understood that this disclosure is not limited to particular biological systems, which can, of course, vary.

Furthermore, wherever feasible and convenient, like reference numerals are used in the figures and the description to refer to the same or like parts or steps. The drawings are in a simplified form and not to precise scale. It is understood that the disclosure is presented in this manner merely for explanatory purposes and the principles and embodiments described herein may be applied to devices and/or system components that have dimensions/configurations other than as specifically described herein. Indeed, it is expressly contemplated that the size and shapes of the composition and system components of the present disclosure may be tailored in furtherance of the desired application thereof.

Various techniques and mechanisms of the present disclosure will sometimes describe a connection or link between two components. Words such as attached, linked, coupled, connected, fused, and similar terms with their inflectional morphemes are used interchangeably, unless the difference is noted or made otherwise clear from the context. These words and expressions do not necessarily signify direct connections, but include connections through mediate components and devices. It should be noted that a connection between two components does not necessarily mean a direct, unimpeded connection, as a variety of other components may reside between the two components of note. Consequently, a connection does not necessarily mean a direct, unimpeded connection unless otherwise noted.

Similarly, the phrase “operatively linked” as used herein refers to elements or structures in a nucleic acid sequence or amino acid sequence that are linked by operative ability and not physical location. The elements or structures are capable of or characterized by, accomplishing a desired operation. It is recognized by one of ordinary skill. In the art that it is not necessary for elements or structures in a nucleic acid sequence to be in a tandem or adjacent order to be operatively linked.

Unless defined otherwise, all technical and scientific terms used herein have the same meaning as commonly understood by one of skill in the relevant arts. Although any methods and materials similar to or equivalent to those described herein can be used in the practice or testing of the subject of the present application, the preferred methods and materials are described herein. Additionally, as used in this specification and the appended claims, the singular forms “a”, “an” and “the” include plural referents unless the content clearly dictates otherwise. Thus, for example, reference to “an RNA” includes a combination of two or more RNAs; reference to “bacteria,” unless otherwise specified, includes mixtures of bacteria, and the like.

The term “about,” as used herein, means approximately, in the region of, roughly or around. When the term “about” is used in conjunction with a numerical range, it modifies that range by extending the boundaries above and below the numerical values set forth. In general, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also t be understood that all numbers and fractions thereof are presumed to be modified by the term “about.”

The terms “polypeptide,” “peptide,” and “protein” are used interchangeably herein to refer to a polymer of amino acid residues, a polypeptide, or a fragment of a polypeptide, peptide, or fusion polypeptide. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The term “amino acid” refers to naturally occurring and synthetic amino acids, as well as amino acid analogs and amino acid mimetics that function in a manner similar to the corresponding naturally occurring amino acids. Naturally occurring amino acids are those encoded by the genetic code, as well as those amino acids that are later modified, e.g., hydroxyproline, γ-carboxyglutamate, and O-phosphoserine. “Amino acid analogs” refer to compounds that have the same basic chemical structure as a naturally occurring amino acid, i.e. a carbon that is bound to a hydrogen, a carboxyl group, an amino group, and an R group (e.g., homoserine, norleucine, methionine sulfoxide, methionine methyl sulfonium). Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

“Nucleic acid” refers to deoxyribonucleotides or ribonucleotides and polymers thereof in either single- or double-stranded form and complements thereof. The term encompasses nucleic acids containing known nucleotide analogs or modified backbone residues or linkages, that are synthetic, naturally occurring, and non-naturally occurring, have similar binding properties as the reference nucleic acid, and metabolized in a manner similar to the reference nucleotides. Nucleotides may be referred to by their commonly accepted single-letter codes.

The term “interaction domain” refers to peptides or proteins (that may be glycosylated or otherwise modified), that are adapted to specifically interact with target regions (or targets) on other molecules differing from themselves. For example, and without limitation, a viral coat protein may comprise an interaction domain that is adapted to specifically interact with an origin of self-assembly (i.e. the target) as defined below.

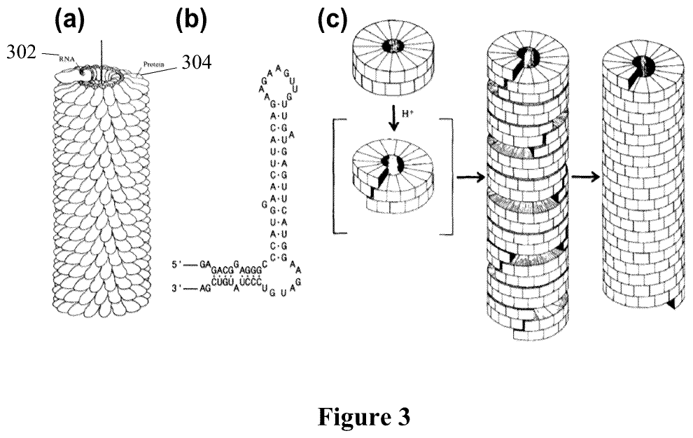

As used herein, the terms “origin of self-assembly,” “origin of assembly,” and “OAS” each refer to an internal RNA stem-loop sequence present in the viral RNA genome that is adapted to interact with viral coat proteins of the virus or other interaction domains to form one or more structures having a substantially defined geometry and including three (3) or more units. An OAS may be the target for disk binding in assembly initiation and may be specifically recognized by the viral coat protein disk aggregate (see FIG. 3). For example, with respect to viruses, and TMV in particular, a TMV coat protein may be the interaction domain and interact/bind with the origin of assembly to spontaneously form an initiation complex to which additional subunits can rapidly bind to create a hairpin loop or other stacking formation. An origin of self-assembly may be connected to the interaction domain by a linker region (also referred to herein as a “linker” or “linker sequence”).

The phrase “derived from” refers to a component that is isolated from or made using a specific molecule or organism, or information from a specific molecule or organism. As such, as used herein, the phrase “derived from genetic material encoding” refers to something that includes a peptide or protein which could have been substantially produced by transcription of DNA and/or translation of RNA encoding that peptide or protein, or a larger protein of which it forms a part, followed if necessary by cleavage (natural or unnatural) and/or post-translational modification. It will be apparent that a peptide or protein will be derived from genetic material even if the actual genetic material encoding it differs through degeneracy in the genetic code or conservative substitution or the like. Similarly, a DNA or nucleotide “coding sequence” or “sequence encoding” a particular polypeptide or protein refers to a nucleic acid sequence that is transcribed and translated into a product (e.g., a polypeptide or protein) when placed under the control of appropriate regulatory sequences.

As used herein, the term “encodes” refers to any process whereby the information in a polymeric macromolecule or sequence string is used to direct the production of a second molecule or sequence string that is different from the first molecule or sequence string. As used herein, the term is used broadly and can have a variety of applications. For example, as is well known in the art, a DNA molecule can encode an RNA molecule (e.g., by the process of transcription incorporating a DNA-dependent RNA polymerase enzyme). Also, an RNA molecule can encode a peptide, as in the process of translation. When used to describe the process of translation, the term “encode” also extends to the triplet codon that encodes an amino acid. In some aspects, an RNA molecule can encode a DNA molecule, e.g., by the process of reverse transcription incorporating an RNA-dependent DNA polymerase. In another aspect, a DNA molecule can encode a polypeptide, where it is understood that “encode” as used in that case incorporates both the processes of transcription and translation.

As used herein, the term “isolated” means that the material is removed from its original environment, e.g., the natural environment if it is naturally occurring. For example, a naturally occurring polynucleotide or polypeptide present in a living organism is not isolated, but the same polynucleotide or polypeptide separated from some or all of the coexisting materials in the natural system is isolated. Such polynucleotides could be part of a vector and/or such polynucleotides could be part of a composition and remain isolated in that such vector or composition is not part of its natural environment.

Unless otherwise expressly stated, the term “purified” and the like does not necessarily require absolute purity has been achieved; rather, it is intended as a relative definition that relates to enrichment of a molecule or compound relative to other components normally associated with the molecule or compound in a native environment.

“Plasmids” are designated herein by a lower-case p preceded or followed by capital letters and/or numbers. The starting plasmids described in the present disclosure are commercially available, publicly available on an unrestricted basis, or can be constructed from available plasmids in accordance with published procedures. Additionally, equivalent plasmids to those described herein are known in the art and will be apparent to one of ordinary skill in the art. For example, while pET21 is used in the examples set forth below, it will be appreciated that any suitable plasmid or other expression vector now-known or hereinafter discovered may be utilized to achieve like results (for example, and without limitation, pETM6, pBAD24, etc.).

As used herein, the phrase “a functional equivalent” of a sequence means a nucleic acid or amino acid sequence that has greater than 40% homology with the nucleic acid or amino acid sequences (respectively) referenced and that has essentially the same properties, structure, and/or functionality. Accordingly, with respect to nucleic acid sequences, “functional equivalents” may include codon variants that ultimately produce the same protein. A number of studies have demonstrated that the functional equivalents of nucleic acid sequences can be prepared by maintaining the base pairing of most of the double helical regions even when changes are made to the stems. Changes in the stem sequence does not significantly affect secondary structure and/or the functionality of the underlying structure; therefore, 100% sequence identity is not required in all cases to achieve the desired structure and functionality of the resulting molecule. Similarly, in proteins, amino acid exchange can occur while still preserving protein function, for example if the modifications occur in specific regions within a protein that are not important for its function and/or if positional homology is preserved. Here, preferably, functional equivalents include those sequences having an identity of at least 70%, 75%, 80%, 90%, 97%, 98%, 99% or more and maintains the structure and functionality of the original. Of course, “functional equivalents” also encompasses fragments, in particular individual domains or sequence motifs, of the proteins and polypeptides of the present disclosure which have the desired biological activity such as, for example and without limitation, self-assembly.

As used here, the phrase “a structure having substantially defined geometry” means a structure the approximate size and shape of which is consistent when it is formed from the same components under the same conditions.

Further, in the context of the present disclosure, a “nanoparticle” is considered to be a particle having at least one dimension less than about 150 nm. For example, a Barley stripe mosaic viron nanoparticle may have a diameter at or about 18 nm, but a length of about 100-150 nm. In certain embodiments, the nanoparticle can be less than 100 nm in every dimension.

The term “virus particle” as used herein means any non-enveloped virus particle (VP) whether or not infectious, including virus-like particles that lack nucleic acid content. Exemplary embodiments of suitable VPs in the present disclosure include non-enveloped viruses having a capsid coat (for example, and without limitation, a rod-shaped (helical) capsid). Exemplary examples of rod-shaped viruses include the Barley stripe mosaic virus (BSMV).

As used herein, the terms “virus-like particles” and “VLPs” refer to molecules that closely resemble viruses yet are non-infectious because they contain no viral genetic material as mentioned above. As described below, VLPs can also be used as a nanotemplates, whether they are naturally occurring or synthesized through the individual expression of viral structural proteins, which can then self-assemble into the virus-like structure. Combinations of structural capsid proteins from different viruses can be used to create recombinant VLPs and/or proteins, nucleic acids, or small molecules may be attached to the VLP surface.

Metals suitable for use in any process according to the present disclosure include certain salts of metals as well. Particular examples of suitable metals include silver, gold, iron, copper, indium, platinum, palladium, rhodium, manganese, zinc, cobalt, Au/Pd alloy, and the like. Optionally, the metal salts can be salts of silver, gold, iron, copper, indium, platinum, palladium, rhodium, manganese, zinc, cobalt, Au/Pd alloy and the like.

The present disclosure provides novel nanoparticles and methods for synthesizing the same using VLPs in a microbial expression system. In at least one embodiment, Barley stripe mosaic virus (BSMV) coat proteins or capsids (BSMV-CP) are fused to an OAS, optionally via a linker region, and the transcript is inserted into a plasmid or other expression vector/modality that is then transformed into or otherwise expressed in a host bacterial cell. The transformed cells propagate, which produces the rod-shaped BSMV-VLPs of interest that self-assemble due to the presence of the OAS. Following isolation and purification steps, these bacterial produced BSMV-VLPs may then be employed as biotemplates in the synthesis of nanomaterials (e.g., palladium nanomaterials via hydrothermal methods). To date, only in-planta production methods for BSMV have been achieved; the present disclosure is the first instance successfully engineering BSMV-CP for production in a microbial expression system which allows for engineering heretofore not possible in-planta due to the evolutionary pressures and constraints of such platforms.

In alternative embodiments, instead of (or in addition to) fusing an OAS with the BSMV-CP to initiate self-assembly, the sequence of the BSMV-CP is engineered to optimize the strength of interaction between capsid protein subunits thereon, which notably has been determined to drive spontaneous self-assembly of BSMV-VLPs when fabricated in a microbial-based expression system. The bacteria assembled BSMV-VLPs have since been successfully used as biotemplates to synthesize organic-inorganic nanomaterials of high quality in the absence of external reducing agents.

Accordingly, the inventive methods disclosed herein produce non-infectious BSMV VLPs using a bacterial protein expression system without the restrictions of conventional BSMV in planta production. These methods and templates are highly biodiverse and amendable to genetic engineering. Indeed, the BSMV-based biotemplates provide a wide range of chemical interactions afforded by its numerous multifunctional protein surfaces. The presence of various functionalities on a single template allow for the formation of a wide range of inorganic nanoparticles therefrom. Additionally, BSMV in particular allows for a vast array of genetic modifications through which enhanced properties can be imparted to the resulting nanoparticles (e.g., accelerated deposition rates) and opens the door to unique nanosynthesis opportunities. Likewise, the use of the microbial production platform in the biotemplate synthesis methods disclosed herein enables protein engineering that is simply not possible in planta due to the evolutionary pressures.

Still further, the biotemplates of the present disclosure (whether BSMV-based or otherwise) exhibit an increased stability over a wider range of conditions than can be achieved using conventionally templates. This is beneficial for numerous applications, particularly in the production of metal and/or metalized nanoparticles and allows for the deposition of new metals with their own distinct properties. Especially when considered with BSMV's enhanced ability to accommodate more metal coating than other viral platforms, this is commercially significant. (As described below, BSMV can adsorb more than twice the amount of metal relative to the current plant viral standard (TMV), which can lead to thicker coatings.) Further, through the incorporation of customizable linkers into the BSMV-CP transcript, the present methods allow for the lengths of the BSMV-derived VLPs to be specifically tailored pursuant to preference or application.

The present disclosure provides an easy and cost-effective solution for biotemplate and high-yield nanoparticle production. When the benefits of the presently disclosed approaches are taken together, it is clear the novel nanoparticles, platforms and methods disclosed herein are a significant advancement in the field.

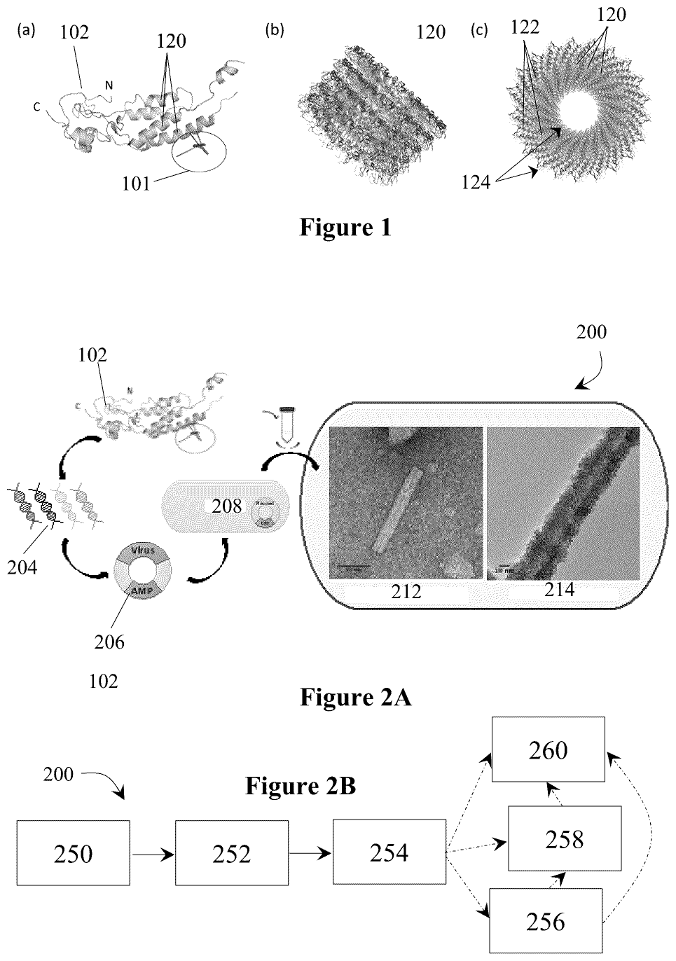

BSMV has recently been proposed as an attractive template for nanomaterial direct synthesis as it shows at least two-fold higher nanoparticle adsorption capability than that of the popular TMV. BSMV virons are rigid rods consisting of a tripartite positive sense ssRNA genome surrounded by virus coat or capsid proteins (CPs) of 23 kDa. The particles (virons) are about 20.8 nm to about 21.4 nm in outer diameter, with an inner central channel of about 4 nm, and between about 110-150 nm long (although particles are known to align end-to-end to produce much longer rods). The BSMV CP tertiary structure, which is shown in subpart (a) of FIG. 1, is similar to that of TMV CP in that both have the presence and positioning of major alpha helices and conservation of key amino acid residues. The N- and C-terminals of the BSMV capsid protein 102 (SEQ ID NO: 1) are labeled as N and C, respectively in subpart (a) of FIG. 1. BSMV CPs 102 assemble around an interacting RNA 101 to form a viron (or particle) that retains a structural integrity relevant for infectivity up to about 65° C. after about 10 min. Consequently, BSMV can potentially be applied to surface biomineralization, while retaining its mechanical robustness, as has been observed on TMV. FIG. 1 also shows a side view of an assembled BSMV viron in subpart (b), with six turns around the RNA 101 visible, and a top-down view of a single turn of assembled BSMV capsid proteins (without RNA 101) in subpart (c). Green tubes 120 indicate Alpha helices with N-C terminal direction. Blue wires 122 are protein side chains and the red and blue regions 124 indicated in subpart (c) of FIG. 1 represent local positively and negatively charged regions of the capsid protein, which are notably on the outside or inner hollow of the virus. The structure shown in FIG. 1 was obtained by cryo-EM and visualized by pymol and NGL viewer, a web-based molecular graphics for large complexes.

BSMV offers alternative biotemplating due to its unique physiochemical properties (e.g., isoelectric point) and other active surface functionalities, which allow for different chemical interactions as compared to TMV. For example, the BSMV-CP 102 consists of two additional long insertions on the outer surface when compared to the TMV-CP. One of these insertions is a sequence of 10 amino acids (residues 1-10), located at the exposed N-terminus, while the other (residues 84-94) is an insertion loop that also protrudes from the outer surface. This second region (residues 84-94) in particular, provides significant opportunity for genetic modifications to incorporate desired properties such as, by way of a non-limiting example, accelerated deposition rate.

Recently, the investigators' studies demonstrated successful synthesis of palladium nanorods by using in-planta produced BSMV as an alternate template to TMV (FIG. 1). The synthesized nanorods were of similar, if not higher, quality than those produced with TMV and BSMV was shown to adsorb at least twice as much metal as TMV, thus leading to thicker coatings and unique nanosynthesis opportunities.

However, conventional approaches to produce BSMV are limited to in-planta production, which necessarily limits the ability to genetically engineer and mass produce the virus. First, as noted above, the genomes of inplanta-synthesized viruses, and BSMV in particular, are associated with mutations and recombination during viral replication due to evolutionary pressures that may remove any desired engineered modification in the interest of viral fitness. Second, conventional BSMV in planta production utilizes infectious plant pathogens that leverage the viral replication cycle in plants, which requires an extended period of time before a relatively small quantity of viruses can be extracted.

Unlike conventional in planta methods, the production methods of the present disclosure use microbial expression platforms for nanoparticle production, which are fast, simple and result in high yields. VLP self-assembly of wild type virus coat protein is initiated by the OAS. However, the OAS of BSMV is unknown. Accordingly, heretofore, BSMV-VLPs have not been produced in bacteria due to their inability to self-assemble from wild-type BSMV. The novel methods provided herein provide self-assembly functionality to BSMV-VLPs, thereby allowing for the beneficial use of microbial expression platforms in this context. Furthermore, these methods uniquely offer the ability to tune the length of the VLPs as desired or needed for a particular application.

Now referring to FIGS. 2A and 2B, at least one embodiment of a novel method 200 for producing BSMV-VLPs in a microbial expression system is shown. This method 200 will be first be explained in general terms to aid in understanding, followed by a more detailed descriptions of various aspects and using specific examples. Notably, method 200 is the first method that allows for the length of BSMV-derived VLPs to be specifically tailored as desired, thereby allowing for the ability to produce biotemplates with a customized morphology for use as a biotemplate to synthesis organic-inorganic nanomaterials.

Generally, in at least one embodiment, the method 200 comprises the steps of constructing a plasmid or expression vector comprising a fusion of a viral CP and an OAS 302 (step 250), transforming such plasmid or expression vector into a host and expressing the same using an expression system 208 (step 252), and isolating the resulting VLPs 212 from the expression system (step 254).

Method 200 may optionally further include the step of nanoparticle synthesis (step 260) using the VLPs 212 produced at step 254 and, where desired, one or more interim steps such as coating at least a surface of the resulting VLPs 212 with a metal using adsorption or the like (step 256) and/or performing microbial reduction of the VLPs (step 258) prior to nanoparticle synthesis (step 260).

In at least one embodiment, the expression system 208 is heterologous and may comprise an Escherichia coli (E. coli) platform. While E. coli is the host platform described herein, it will be appreciated that any number of expression systems may be utilized in the present method 200 including, without limitation, S. cerevisiae or those non-bacterial expression systems that utilize insect cells and/or mammalian cells. Furthermore, DNA of the novel CP-OAS disclosed herein may be integrated into a genome for expression.

Additionally or alternatively, the viral CP/interaction domain may be a BSMV-CP 102 or any other virus strain from which a suitable CP can be produced and assembled into a VLP 212 using the method without the presence of a naturally occurring OAS therein. In at least one embodiment of the present disclosure, the viral CP/interaction domain may be of any viral strain that does not include a native OAS such as, for example, other rigid, rod-shaped viruses in the Hordeivirus genus or those in the Furovirus, Pecluvirus, Pomovirus, Tobamovirus, or Tobravirus geneses in the family of Virgaviridae.

As previously noted, one of the hurdles to using BSMV is that native BSMV coat protein transcripts lack the ability to self-assemble into VLPs (i.e. initiate the assembly from disk to rod structure). As shown in FIG. 3, CP units in certain viruses (like TMV) spontaneously form a disk structure (represented in subparts (a) and (c) of FIG. 3) by the binding of an OAS 302 into the central hole of a two-ring subassembly of the capsid protein 304. More specifically, the interactions of secondary-structured OAS with CP units drive the spontaneous nucleation of disk subassembly, with these “disks” forming in long aggregates that are made up of discrete short helices (corresponding in size to the number of subunits in the disk). This consequently leads to self-assembly of disk structures into a helical rod-shaped particle, with the disks initially stacked in imperfect register, but annealing over time into a helical rod-shaped particle (see subpart (c) of FIG. 3).

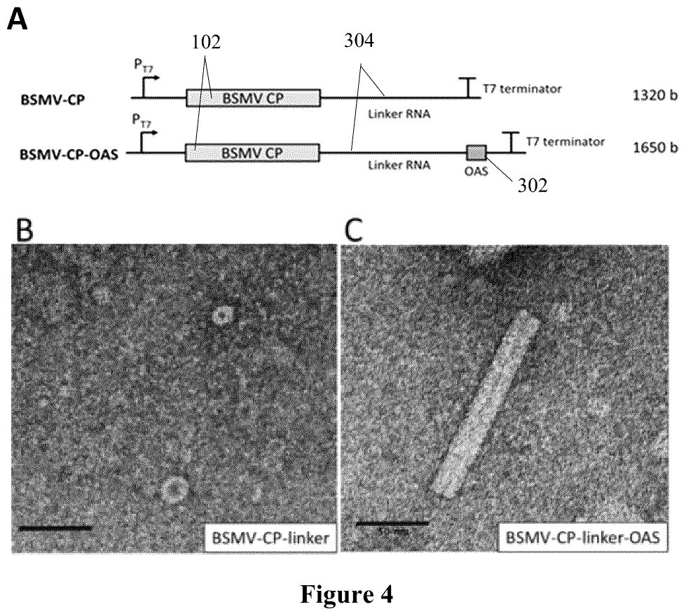

When a BSMV-CP transcript is expressed alone there is no assembly. To address the inability of native BSMV to self-assemble, at step 250, a plasmid or other expression vector is constructed comprising a fusion of an interaction domain such as a viral CP (e.g., BSMV-CP 102, as shown in FIG. 2A) and an OAS 302 (SEQ ID NO: 26). The OAS 302 sequence may be operatively linked to the BSMV-CP sequence such that a resulting product is a BSMV-CP operatively linked with an OAS 302; indeed, in at least one exemplary embodiment, the OAS 302 is introduced downstream into the protein transcript as shown in FIG. 4, subpart A.

The operative linkage between the interaction domain and the OAS 302 may be direct fusion or via a linker 304 (described in further detail below). In at least one exemplary embodiment, BSMV-CP is prepared with an OAS 302 from TMV at the 3′ end derived from SEQ ID NO: 11. It has been determined that the inclusion of OAS 302 in the construct initiates self-assembly via the RNA/CP interaction with the BSMV-CP. Further, the plasmid and/or expression vector may be optimized for bacterial expression pursuant to protocols known in the art.

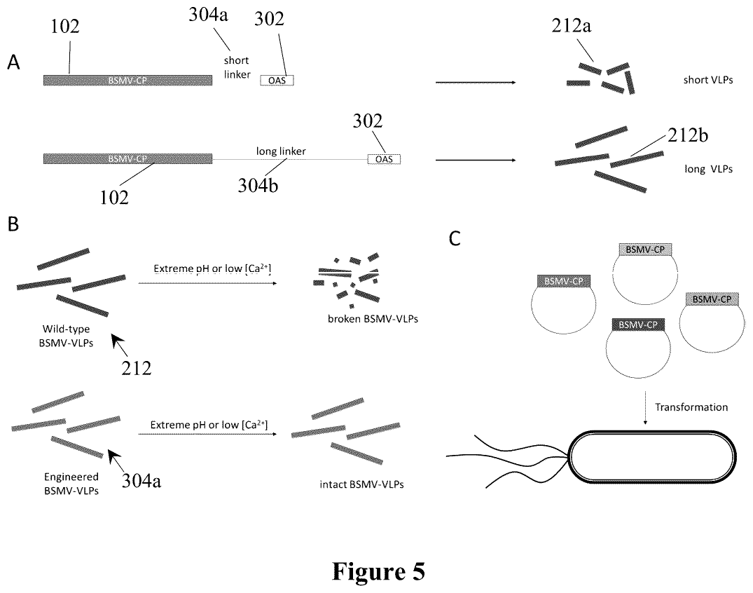

Now referring to FIG. 5, as noted above, the interaction domain (e.g., BSMV-CP 102) may be linked to the OAS 302 via a linker region 304 according to protocols known in the art such as, and without limitation, construction techniques such as DNA synthesis or gene splicing by overlap extension polymerase chain reaction (PCR) (for example, the linker 304 may comprise a fusion protein). Linker regions 304 may be selected from any number of peptide sequences, nucleic acid sequences, or other suitable materials. In at least one exemplary embodiment, the linker region 304 has a nucleic acid sequence of SEQ ID NO: 13. The length of a linker region 304 will depend on several factors, including the geometry of the self-assembly units and the desired morphology of the resulting VLPs. It is generally desirable to provide a linker region 304 of sufficient length to allow the interaction domain operatively connected to the self-assembly unit to orient towards its target, thus permitting sufficient binding. In at least one embodiment, the length of a linker region 304 may be selected to allow for maximum target accessibility to the binding sites of the interaction domain. It may further be desirable in certain applications to select linkers for resistance to proteases (e.g., where in vivo applications are contemplated). In at least one exemplary embodiment, the linker region 304 is positioned downstream of the CP, after a stop codon and before the OAS 302 as shown in FIG. 5, subpart A.

In some instances, it may be desirable to use linkers 304 between about 600 and about 700 nucleic acids in length and in other instances the use of linkers 304 between about 2,200 and about 2,300 nucleic acids in length may be desirable. In at least two exemplary embodiments, the linker region 304 may be 661 or 2,243 nucleic acids in length. It will be appreciated that these linker lengths are provided solely by way of example and in no way limiting; the linker region 304 may comprise any length suitable or desired for a particular application.

Linkers 304 may also be used to join a marker (e.g., such as a fluorescently labeled moiety or compound) or a destructive material (e.g., a radioactive material of sufficient activity) to a self-assembly unit. In at least one embodiment, the linker 304 may be secured to the opposite terminus of the self-assembly unit from the interaction domain.

Incorporation of a linker region 304 imparts the ability to tune and/or modify the length of the resulting noninfectious VLP 212 and, thus, any nanoparticle subsequently synthesized therewith. Indeed, it has been determined that length of the overall construct directly correlates to the length of any resulting VLP 212 produced at step 252.

Subpart A of FIG. 5 illustrates this concept. Use of a linker 304a having a first, shorter sequence (for example, and without limitation, a 100 amino acid linker), results in VLPs 212a having a corresponding first, shorter length, whereas use of a second, longer linker 304b sequence (for example, and without limitation, a 1322 amino acid linker) results in VLPs 212b having a corresponding second, longer length. This property can be exploited by strategically choosing the linker length to tune the morphology of the VLPs 212 produced.

It should also be noted that any desired engineering to BSMV may be performed at or prior to step 250 to take advantage of the transformation and expression step 252. For example, because surface residues of BSMV can be modified, in at least one embodiment, BSMV-VLPs can be conjugated with antigen display for medical applications. There, the resulting BSMV-VLPs would function as a vaccine scaffold to elicit a desired immune response following administration to a subject, such as, for example, the L2 protein fragment from the papillomavirus does when conjugated with TMV.

At step 252, the constructs are transformed into a host expression system (such as a microbial-based expression system comprising E. coli, for example) and grown such that the construct is expressed and VLPs 212 are produced. SEQ ID NO. 26 provides a nucleic acid sequence of one such E. coli plasmid carrying a BSMV-CP 102 fused with a linker region 304 and an OAS 302. In at least one embodiment, the E. coli transformed with the plasmids or expression vectors were grown at room temperature for 16-20 hours. FIG. 4, subparts B and C shows images taken using a 200 kV Tecnai T20 transmission electron microscope (TEM) of lysed BSMV-CP-linkers at step 254, both with (subpart C) and without (subpart B) an OAS 302 fused therewith. As is clearly seen in subpart B of FIG. 4, the BSMV-CP-linker without an OAS 302 failed to self-assemble during step 252 (only disks structure formed without OAS 302), whereas the image of the BSMV-CP-linker fused with an OAS 302 indicates the presence of self-assembled, rod-shaped BSMV VLPs indicating that self-assembly did in fact occur at step 252. Accordingly, at step 250, a TMV-OAS 302 is fused to the BSMV-CP 102 transcript using methods known in the art, which imparts self-assembly characteristics into the BSMV-VLPs 212 thereafter produced at step 252 through the expression system 208.

Unlike bacteriophage systems such as M13 that infect the bacterial platform and limit options for property customizations, plant viruses can be expressed heterologously without affecting the producing bacteria. In other words, because VLP production is independent of a virus's ability to infect or alter microbial function, the heterologous expression system 208 utilized at step 252 allows for more opportunities to engineer VLP properties without compromising properties and quality in E. coli bacteria by infection. Further, because a heterologous host is employed, the evolutionary pressures on virus replication are reduced as compared to in-planta models, which further promotes the capability to genetically engineer the VLP structures. Accordingly, by employing the powerful and unique abilities of synthetic biology, method 200 utilizes a heterologous expression system such as an E. coli platform to produce VLPs with genetic modifications that plant hosts are not able to achieve.

At step 254, the resulting VLPs 212 are isolated from E. coli and purified pursuant to protocols known in the art. The VLPs 212 may be used as biotemplates for the synthesis of nanoparticles at step 260 pursuant to known methods. In the embodiment utilizing BSMV-VLPs 212 as described herein, nanosynthesis results in the production of high quality nanorods having size and dimensions that correlate with those of the VLPs 212. Where a linker region 304 was employed in the construct at step 250, the custom VLPs 212 will have a size and dimension that directly correlates with the length of the customized linker region 304.

Optionally, prior to step 256, the VLPs 212 may be coated with metal at step 256. Metal coated biotemplates have numerous commercial uses. For example, as a component in batteries such as electrodes, chemical sensors, and memory devices, as well as catalysts. It has been determined that metal-coated TMV increases the charge capacity of an anode ten-fold via increasing the surface area thereof. Because wild-type BSMV has more than two-fold metal coating ability as compared to TMV, BSMV-VLPs have the potential to further boost the capacity of batteries over conventionally attainable standards.

Alternative embodiments of method 200 may utilize BSMV-CP transcripts that do not necessarily contain the OAS 302 at all (native or engineered). Instead, in such embodiments, the transcripts are engineered at step 250 with one or more specific point-mutations in the BSMV-CP to optimize the strength of interaction between the CP subunits thereof to result in a more stable biotemplate/VLP.

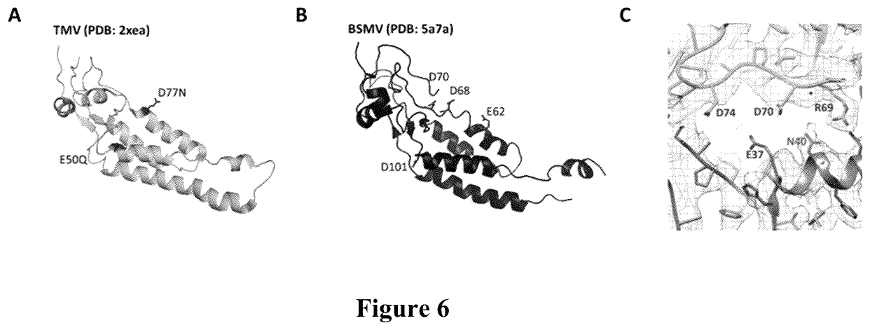

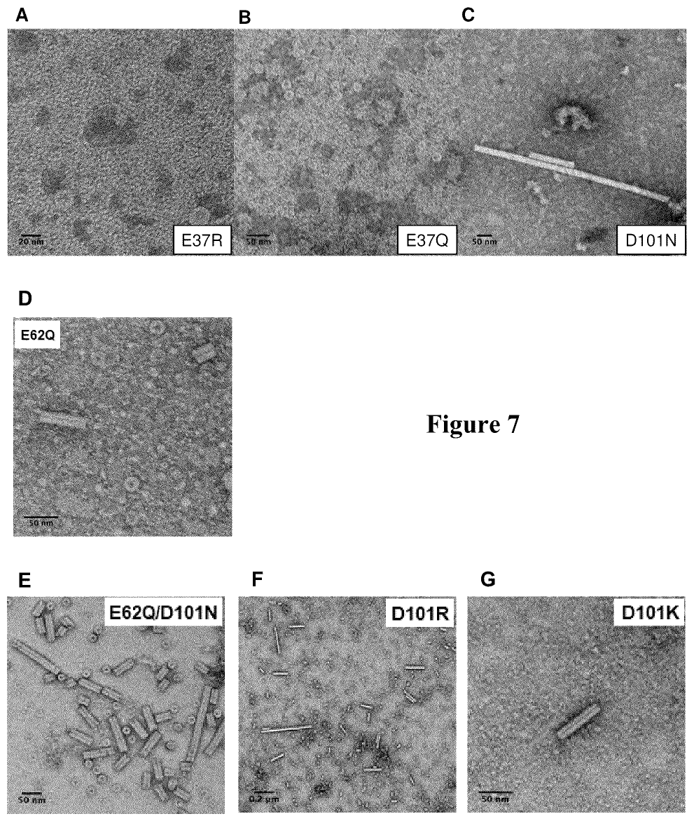

To stabilize these interactions, one or more individual point-mutations can be made in the BSMV-CP using site-directed mutagenesis or the like to neutralize or change the targeted residue to the opposite charge to strengthen the interaction between subunits (see subpart C of FIG. 5) pursuant to methods known in the art. Based on crystal structures, the present investigators have identified E37Q, E37R, E62Q, D68N, D70N, and D101N on the BSMV-CP as probable sites that, if mutated as described, enhance the stability of BSMV. FIG. 6, subparts A-G illustrate the interacting residues that are identified targets for site mutations of the TMV-CP subunit (subpart A), those of the BSMV-CP subunit that correspond to the TMV targets (subpart B), and those of BSMV-CP previously identified in the literature. These sites contain charged residues that may be neutralized, for example, by calcium ions to facilitate neutral assembly/disassembly of the virus during infection. By mutating these residues, the interaction therebetween can be strengthened to achieve self-assembly without an OAS 302. Mutations of D101N (SEQ ID NO: 5), E62Q (SEQ ID NO: 9), both D101N and E62Q (SEQ ID NO: 10), D101R (SEQ ID NO: 6), and D101K (SEQ ID NO: 4) in particular have been tested and validated as increasing stability in this manner and allowing for engineered self-assembly pursuant to the methods and systems described herein without an OAS 302 (see FIG. 7).

Further, and importantly, it has been determined that site specific mutations also support the RNA-free (i.e. no OAS) self-assembly of VLPs. FIG. 7 shows a TEM visualization of BSMV-CP fused to an artificial linker carrying a E37R mutation (subpart A; SEQ ID NO: 8), a E37Q mutation (subpart B; SEQ ID NO: 7), a D101N mutation (subpart C; SEQ ID NO: 5), a E62Q mutation (subpart D; SEQ ID NO: 9), both a D101N mutation and a E62Q mutation (SEQ ID NO: 10), a D101R mutation (SEQ ID NO: 6), and a D101K mutation (SEQ ID NO: 4), with the formation of self-assembled nanorods being verified and clearly visible (see subparts C-G of FIG. 7). It should be noted that the linker region 304 is the residual sequence and, without fusion of an OAS, native BSMV-CP does not form VLPs without one or more of the point mutations described herein. Accordingly, the modified mutants exhibited stronger protein-protein interactions as compared to wild-type that stabilized the VLPs and led to self-assembly.

In certain embodiments, the BSMV-CP comprises at least one site-directed mutation at residue E62, D101, or both residues E62 and D101. Each site-directed mutation can, for example, independently comprise a neutral or positive amino acid. The neutral or positive amino acid can comprise glutamine, asparagine, arginine, or lysine, for example. In certain embodiments, the site-directed mutation at residue E62 comprises SEQ ID NO: 9 or a functional variant thereof. In certain embodiments, the site-directed mutation at residue D101 comprises SEQ ID NO: 5 or SEQ ID NO: 6, or a functional variant of either of the foregoing sequences. In certain embodiments, the site-directed mutation at both residues E62 and D101 comprises SEQ ID NO: 10 or a functional variant thereof.

As used herein, a “functional variant” of an amino acid sequence, a nucleic acid sequence, or peptide is an amino acid sequence, a nucleic acid sequence, or a peptide that can provide the same biological function as the reference sequence or peptide. In certain embodiments, the variants have less than 20, 11, 9, 8, 7, 6, 5, 4, 3, or less than 1 amino acid or nucleic acid replacement as compared to the reference sequence or peptide.

Desirably, the sequences maintain about 90% to about 100% identity (e.g., 90% identity to about 100% identity, about 90% identity to 100% identity, or 90% identity to 100% identity), such as about 92.5% identity to about 97.5% identity (e.g., 92.5% identity to about 97.5% identity, about 92.5% identity to 97.5% identity, or 92.5% identity to 97.5% identity), or such as about 95% identity to about 96% identity (e.g., 95% identity to about 96% identity, about 95% identity to 96% identity, or 95% identity to 96% identity). The ranges set forth in this paragraph are inclusive of the stated end points and each 1% increment encompassed therein.

The term “identity” with respect to a reference to an amino acid or polypeptide sequence is defined as the percentage of amino acid or nucleic acid residues, respectively, in a candidate sequence that are identical with the residues in the reference sequence, after aligning the sequences and introducing gaps, if necessary, to achieve the maximum percent sequence identity and not considering any conservative substitutions as part of the sequence identity. Identity is measured by dividing the number of identical residues by the total number of residues and multiplying the product by 100 to achieve a percentage. Thus, two copies of exactly the same sequence have 100% identity, whereas two sequences that have amino acid deletions, insertions, or substitutions relative to one another have a lower degree of identity. Alignment for purposes of determining percent sequence identity can be achieved in various ways that are within the skill of the art, for instance, using publicly available computer software. For example, determination of percent identity or similarity between sequences can be done, for example, by using the GAP program (Genetics Computer Group, software; now available via Accelrys online), and alignments can be done using, for example, the ClustalW algorithm (VNTI software, InforMax Inc.). Further, a sequence database can be searched using the nucleic acid or amino acid sequence of interest. Algorithms for database searching are typically based on the BLAST software (Altschul et al., 1990), but those skilled in the art can determine appropriate parameters for aligning sequences, including any algorithms needed to achieve maximal alignment over the full length of the sequences being compared.

It will be noted that while such BSMV-CP engineering techniques may be used to achieve self-assembly of VLPs 212 without the use of OAS 302 in the construct, it may also be desirable to employ such techniques where an OAS 302 is utilized due to the metal coating benefits associated with such engineering. Indeed, success rates for coating VLPs with metal is largely dependent on environmental parameters such as pH and the presence of cations. These factors can potentially destabilize the template and prevent self-assembly due to the carboxylate interactions between CPs and, thus, lead to random aggregate formation and low yields (see subpart B of FIG. 5). Strengthening the interaction of CP subunits in the interaction domain using the platforms and methods described herein render the resulting VLPs less susceptible to extremes in pH and calcium concentration and allow for effective metal coating under wider processing conditions than as seen with conventional approaches. Thus, the present platforms and methods provide avenues through which BSMV's superior metal coating ability can be leveraged. Furthermore, the VLPs of the present disclosure that are synthesized pursuant to the methods described herein exhibit enhanced stability as compared to conventional biotemplates thus allowing for the synthesis of more homogenous nanoparticles than has been heretofore achieved.

At least in part because VLPs can exhibit highly ordered nanoscale structures with diverse geometries composed of hundreds to thousands of capsid proteins, VLPs can provide an opportunity for high-density surface display. Decoration with small-molecule chemicals, polymers, peptides, proteins, metals, and other ligands can enable the widespread application of plant viruses in bio(sensing), catalysis, energy, and medicine.

As described herein rod-shaped plant viruses such as TMV and BSMV can be used as nanomaterial templates. TMV is composed of a single capsid protein that self-assembles via protein:: protein and nucleic acid:: proteins interactions. Viral capsid proteins can first assemble into washer-like structures with a diameter of about 20 nm via hydrophobic and electrostatic interactions encoded within their structure. These washers can stack into nanorods to encapsidate viral genomic RNA and complete the multi-stage assembly process. Once the rods enter host cells, viral capsid proteins can repel each other to disassemble the rods and restart the viral replication cycle. The transition between self-assembly and self-repulsion can be mediated in TMV by a cluster of negatively charged carboxylate residues within the capsid protein or Caspar Carboxylate Cluster (CCC) motif. These repulsive interactions can be neutralized through proton or calcium ion-mediated charge shielding, allowing for viral assembly. However, under higher pH or low Ca2+, such as that experienced intracellularly, these repulsive interactions are unshielded and the particles disassemble.

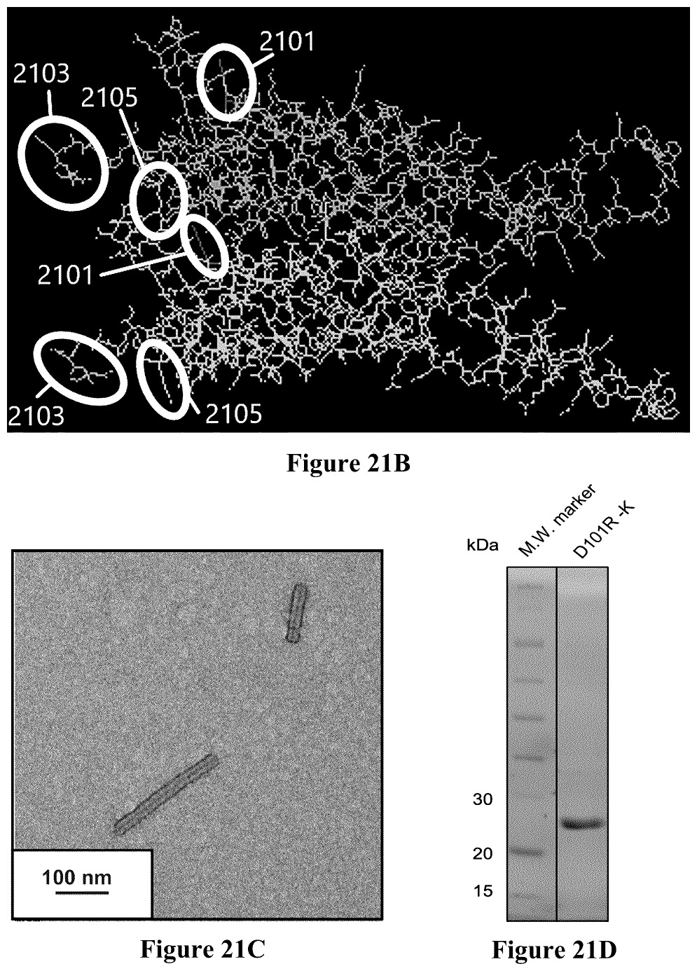

The C-termini of both TMV and BSMV are exposed on the particle surface, which is essential for incorporating functional moieties onto the surface of TMV and BSMV (FIG. 21A). Various chemical and bioconjugation studies have been established for the surface functionalization of rod-shaped plant viral particles, each with unique advantages and disadvantages. Direct fusion to the surface-exposed C-terminus can be the most straightforward route for peptide and protein display, which is common when diverse biological functionalities are desired. However, this approach is heavily restricted by the need to preserve host infectivity for in planta production. Direct fusion is typically limited to short peptides below about 20 residues long. Even the insertion of a single residue can interfere with plant viral replication and stability. For example, significant screening and optimization were used to generate TMV mutants with surface-exposed lysine residues, and all successful clones contained extra acidic residues to balance the positive charge of lysine's amino side chain. Smith et al., Modified Tobacco mosaic virus particles as scaffolds for display of protein antigens for vaccine applications, Virology 348: 475-488 (2006). Once the lysine-displaying mutants were generated, they enabled surface functionalization via chemical coupling and indirect bioconjugation through the binding of streptavidin to biotinylated VLPs. Wege & Geiger, Dual functionalization of rod-shaped viruses on single coat protein subunits, in: C. Wege, G. P. Lomonossoff (Eds.), Virus-Derived Nanoparticles for Advanced Technologies: Methods and Protocols, Springer, New York, NY, pp. 405-424 (2018). Nonetheless, the requirement of plant host infectivity has clearly limited viral engineering to date.

The noninfectious BSMV VLPs hereof, produced in bacteria, overcome these issues and allow for faster production times and decoupling viral fitness or infectivity from production. Lee et al., Bacterial production of barley stripe mosaic virus biotemplates for palladium nanoparticle growth, ACS Applied Nanomaterials 3: 12080-12086 (2020). The bacterially produced VLPs hereof can be used for templating with similar efficiency to inplanta virus and are more amendable to engineering that can introduce desired properties to otherwise reduce infectivity (e.g., pH stability/reduction of infectivity at neutral pHs). Rabindran & Dawson, Assessment of recombinants that arise from the use of a TMV-based transient expression vector, Virology 284: 182-189 (2001). Accordingly, the next steps were to further engineer BSMV VLPs with targeting physicochemical and biological properties.

Engineering rod-shaped plant VLPs with improved stability at alkaline pH can be crucial for important functionalization approaches such as, and without limitation, metal mineralization and chemical modification. For example, buffer pH near and above 9 can greatly accelerate the electroless deposition of ruthenium oxides for catalytic and energy storage applications. This pH range can also favor electroless deposition of other metals and alloys, including nickel-phosphorous. However, since viral particles are typically unstable in this environment, adsorption of these metals typically requires extra processing steps, such as platinum or palladium pre-activation.

Particle instability in alkaline conditions can also be troublesome for functionalization via organic chemical reactions. For example, the electrophilic aromatic substitution of diazonium salts onto tyrosine phenols requires pH values between 9 and 10. This is a popular and highly efficient reaction that can be used to modify rod-shaped plat viruses with diverse functional groups. Similarly, the rate constant can be over 12 times higher at pH 9.5 than at pH 6.5 for the conjugation reaction between various isothiocyanates and a cysteine-functional protein.

Particle surface-exposed lysine residues are another favorable site for chemical couplings to TMV, as the highly nucleophilic amine group can readily attack esters, acids, maleimides isocyanates, and other electrophiles. However, amine nucleophilicity drops dramatically upon protonation, primarily at pH values below its pKa of about 9. Unfortunately, CCC-mediated repulsive interactions can destabilize rod-shaped plant viruses in alkaline conditions far below this value. The enhanced pH stability of the BSMV VLPs described herein therefore enables important engineering opportunities for numerous applications.

Accordingly, provided are BSMV VLPs that exhibit controlled and enhanced self-assembly (e.g., as compared to conventional VLPs), which allows for the ability to maintain the assembly of BSMV capsid protein in a wider range of processing conditions. In certain embodiments, the BSMV VLPs comprise one or more point substitutions of the CCC residues, which enhances the pH stability of the rods by neutralizing repulsive interactions between selected negative carboxylates within the CCC. While a putative CCC for BSMV has been proposed based on crystallization data, the sites have heretofore not been validated. Clare et al., Novel inter-subunit contacts in barley stripe mosaic virus revealed by cryo-electron microscopy, Structure 23: 1815-1826 (2015).

Still further, provided are highly stable VLPs. In certain embodiments, the VLPs hereof are stable at a pH at or between about 4 to about 9. The VLP can comprise one or both: (a) an OAS operatively linked with a BSMV-CP, wherein a portion of the nucleic acid sequence that encodes the OAS comprises SEQ ID NO: 11 or a functional variant thereof, and (b) at least one site-directed mutation on the BSMV-CP at one or more novel CCC residues. The one or more CCC residues can comprise, for example, E62, D101, or both residues E62 and D101. As previously noted, each site-directed mutation can independently comprise a neutral or positive amino acid (e.g., a negatively charged amino acid is replaced with one having a neutral or positive charge). In such instances, the resulting VLP is stable at a pH of at or between about 4 to about 9 (e.g., a pH of 4 to about 9, a pH of about 4 to 9, or a pH of 4-9). The ranges set forth in this paragraph are inclusive of the stated end points and all 0.1 increments included therein. By introducing positive or neutral point mutations on selective carboxylate residues, changes in internal protein-protein interactions can drive the rod-shaped assembly of BSMV capsid proteins and lead to increased nanorod stability.

The VLPs hereof can exhibit versatile pH stability. The VLP can be stable in an acidic environment (e.g., a pH of less than about 7). In certain embodiments, the VLP is stable at a pH of about 4 (such as a pH of 4). In certain embodiments, the VLP is stable at a pH of about 5 (such as a pH of 5). In certain embodiments, the VLP is stable at a pH of about 6 (such as a pH of 6). In certain embodiments, the VLP is stable in a neutral environment (e.g., the VLP is stable at a pH of about 7 (such as a pH of 7)). In certain embodiments, the VLP is stable in an alkaline environment (e.g., a pH of greater than about 7). In certain embodiments, the VLP is stable at a pH of about 8 (such as a pH of 8). In certain embodiments, the VLP is stable at a pH of about 9 (such as a pH of 9). The VLPs hereof can show enhanced structural integrity under atypical processing conditions, for example, at pH values up to about 9.

In certain embodiments, the VLP has a site-directed mutation at residue E62, D101, or both residues E62 and D101 on the BSMV-CP, and each site-directed mutation independently comprises glutamine, asparagine, arginine, or lysine. In certain embodiments, the site-directed mutation at residue E62 comprises SEQ ID NO: 9 or a functional equivalent thereof. In certain embodiments, the site-directed mutation at residue D101 comprises SEQ ID NO: 5 or 6, or a functional variant of either SEQ ID NO: 5 or 6. In certain embodiments, the site-directed mutation is at both residues E62 and D101 and the BSMV-CP comprises SEQ ID NO: 10 or a functional equivalent thereof.

The VLPs can comprise recombinant BSMV VLPs produced via in vivo production. In certain embodiments, the VLPs are engineered for alkaline stability. In certain embodiments, the VLPs hereof are longer than corresponding wildtype BSMV VLPs. For example, the VLP can comprise a rod length of at or between 75 nm-150 nm. In certain embodiments, the VLP comprises a rod length of at or between 80 nm-145 nm. The VLP can comprise a rod length of at or between 85 nm-140 nm. The VLP can comprise a rod length of at or between 95 nm-135 nm. The VLP can comprise a rod length of at or between 100 nm-130 nm. The VLP can comprise a rod length of at or between 105 nm-125 nm. The VLP can comprise a rod length of at or between 110 nm-120 nm. The VLP can comprise a rod length of at about 115 nm. In certain embodiments, the VLPs comprise an average rod length of any of the aforementioned values listed in this paragraph. In certain embodiments, the VLPs comprise an average rod length of about 125 nm (such as 125 nm). The VLPs can comprise an average rod length of 91 nm (such as 91 nm). The ranges listed in this paragraph are inclusive of the stated end points and all 1 nm increments that fall within the stated ranges.

The VLPs hereof can be surface functionalized, for example, via amine couplings. In certain embodiments, a VLP further comprises a surface-exposed C-termini comprising a site-directed insertion of a natural amino acid residue. The natural amino acid residue can be any natural amino acid residue that is not wildtype for the BSMV-CP. The natural amino acid residue can comprise a lysine, glutamic acid, cysteine, tyrosine, aspartate, or glutamate residue. In certain embodiments, the stabilized mutant VLP is modified to display lysine residues at the VLP surface.

The surface exposed C-termini can be functionalized with a ligand. In certain embodiments, the reactive amino functional group of the displayed amino acid residue can be leveraged for chemical modifications. Surface functionalization strategies are well-established for some, but not all, plant viruses. Vaidya & Solomon, Surface functionalization of rod-shaped viral particles for biomedical applications, ACS Applied Bio Materials: acsabm.1c01204 (2022). Popular viruses such as TMV and potato virus X, which claim the majority of research focus on rod-shaped plant viruses in recent decades, have been decorated with a wide array of small molecules, polymers, metals, peptides, proteins, and various other ligands through chemical and biomolecular approaches. The surface of BSMV appears to be more active than TMV, encoding additional electrostatic interactions that result in more dense, rapid, and uniform coating with metals such as palladium. Genetically modifying BSMV VLPs for surface display (e.g., using lysine residue insertions) can facilitate diverse surface modifications through amine chemistry to further develop BSMV VLPs as a biotemplate for nanomaterial synthesis.

The ligand can comprise any ligand known in the art that can be coupled with the surface of the VLPs including, for example, a fluorescent label, an amide, a reactive electrophile, a peptide, a polymer, a small molecule, a metal, or a protein. In certain embodiments, the ligand comprises a therapeutic agent. The term “therapeutic agent” is intended in its broadest meaning to include a compound, chemical substance, microorganism or any agent that is capable of producing an effect in a subject or on a living tissue or cell when administered thereto. Thus, the term includes both prophylactic and therapeutic agents, as well as diagnostic agents and any other category of agent capable of having a desired effect. Therapeutic agents include, but are not limited to, pharmaceutical drugs and vaccines, nucleic acid sequences (such as supercoiled, relaxed, and linear DNA and fragments thereof, antisense constructs, artificial chromosomes, RNA and fragments thereof, and any other nucleic-acid based therapeutic), cytokines, small molecule drugs, proteins, peptides and polypeptides, oligonucleotides, oligopeptides, fluorescent molecules (e.g., fluorophores) and other imaging agents, hormones, chemotherapy, and combinations of interleukins, lectins, and other stimulating agents.

In certain embodiments, the ligand comprises a fluorescamine.

The surface of the VLP can be functionalized using methods known in the art. The surface of the VLP can be functionalized using direct protein fusion. In certain embodiments, the surface of the VLP is functionalized using direct chemical conjugation. Chemical modification of rod viral particles is a popular functionalization strategy with complementary advantages and disadvantages to direct protein fusion. Chemical conjugation is highly modular relative to genetic fusion, which is limited to amino acid chemistry. Chemical methods can be used for functionalization with peptides and proteins and can accommodate large structures. In direct chemical conjugation, functional groups on the ligand of interest can be reacted with the natural, surface-exposed amino acid residues of the VLP (e.g., presented at the surface facing C-termini via the site mutation). Performing these reactions with previously assembled particles decouples the self-assembly process from functionalization. This has the advantage of preventing interference between the ligands and particle assembly. Click chemistries can be employed and can be useful due to their rapid kinetics and near quantitative conversion. For example, thiols can selectively undergo radical-mediated click chemistry with alkenes.

Table 1 lists several reactions that are possible between diverse functional ligands and residue chemistries that may be incorporated on the surface of the VLPs hereof.

TABLE 1
Chemical Coupling Strategies for Rod VLP Functionalization
via Direct and Indirect, Biorthogonal Chemistry

| Reaction / | |
|---|---|
| Residue | Reaction Scheme |
| amide coupling/ glutamic acid | 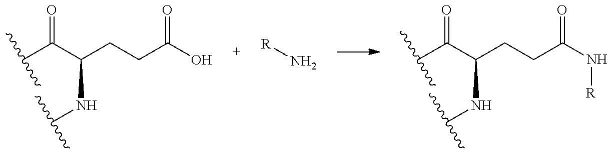 |
| amide coupling/ lysine | 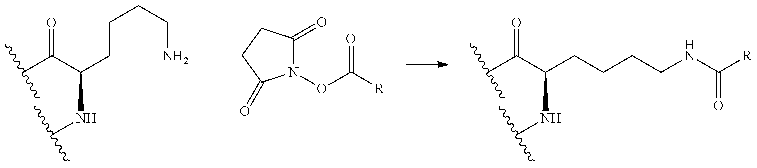 |
| thiol-Michael addition/ cysteine | 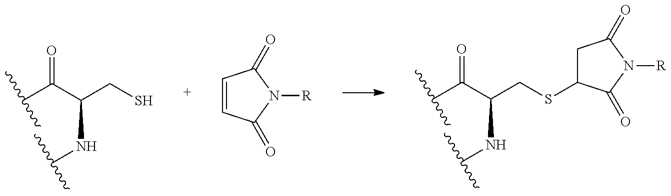 |
| diazonium salt coupling/ tyrosine | 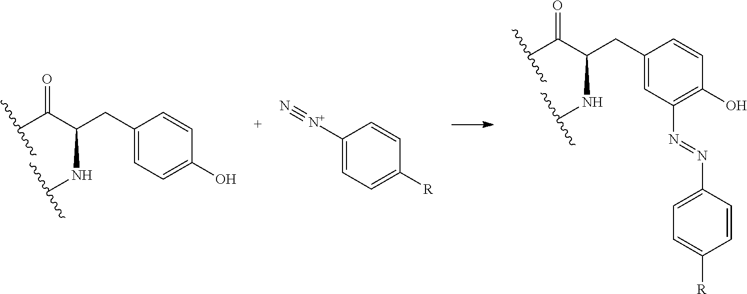 |
| oxidative coupling/ proline | 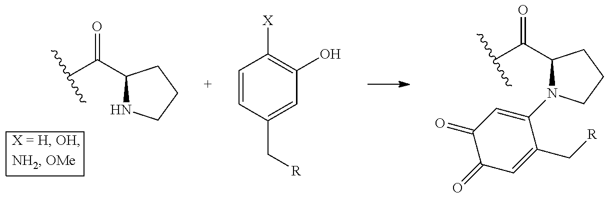 |
| oxime formation | 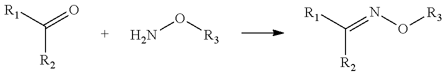 |
| hydrazone formation | 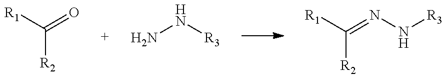 |
| azide alkyne cycloaddition | 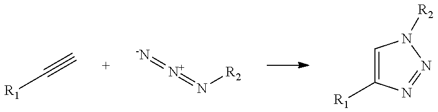 |

Acidic residues aspartate and glutamate found on the surface of the VLPs can be functionalized, for example, by carbodiimide-mediated amide coupling. This chemistry can be used to able VLPs via covalent modification with small molecule amines, including fluorescent reporters such as rhodamine B, for example. Fluorescent labeling can enable particle tracking in cells and animals, and/or to probe the influence of particle aspect ratio on intracellular trafficking in mammalian cells. Aspartate and glutamate can also react with hydroxyl groups on alcohols and other biomolecules including the amino acids serine and threonine. Acidic residues can also coordinate metals and can be used to encapsulate platinum-containing anticancer drugs, for example. Amine and thiols, present in lysine and cysteine residues respectively, can be useful reactive groups to include at the C-termini of the VLPs for display at the surface thereof. These nucleophiles can react with reactive electrophiles including acids, acrylates, maleimides, and N-hydroxy succinimide (NHS) esters. Surface exposed amines can be used in amide coupling with acids, with the conjugation of viral surface-exposed acidic residues to four proteins: outermembrane protein A, the bacterial chaperone DnaK, dihydrolipoamide succinyl transferase, and the major membrane protein Tul4, for example.

Lysines are also reactive with NHS esters, and NHS-terminal PEG chains of varying length and degree of breaching can be conjugated, for example, to surface-exposed lysines (i.e., wherein the C-termini of the VLPs hereof comprise site-directed insertion of lysine). In certain embodiments, the VLPs hereof can comprise site-directed insertion of both lysine and cysteine at the C-termini thereof for surface display.

Indirect chemical approaches can also be used for functionalization. In certain embodiments, highly reactive functional groups can be introduced onto the natural amino acid residues at the C-termini of the VLPs chemically to enable subsequent click reactions with various chemicals of interest. For example, surface exposed lysine amines can be first reacted with a maleimide-functional protected thiol, followed by subsequent deprotection by hydroxylamine to produce new thiol groups on the rod VLP surface, which can then be clicked with a ligand of interest (e.g., a therapeutic agent or a targeting ligand).

Indirect bioconjugation and stimuli-responsive surface functionalization can also be employed to functionalize the VLPs hereof pursuant to protocols known in the art.

Methods for manufacturing a nanoparticle biotemplate are also provided. In certain embodiments, the method of manufacturing a nanoparticle biotemplate comprises: introducing into an isolated host a nucleic acid sequence that encodes a BSMV-CP and one or both of: (a) an OAS operatively linked with the BSMV-CP wherein a portion of the nucleic acid sequence that encodes the OAS comprises SEQ ID NO: 11 or a functional equivalent thereof, and (b) at least one site-directed mutation on the BSMV-CP at residue E62, D101, or both residues E62 and D101, wherein each site-directed mutation independently comprises a neutral or positive amino acid. The method further comprises expressing the nucleic acid sequence in a microbial expression system to produce self-assembled BSMV VLPs and isolating the BSMV VLPs from the microbial expression system. The resulting BSMV VLPs can be stable at a pH of at or between 4-9. The resulting BSMV VLPs can comprise a higher average rod length as compared to wildtype BSMV VLPs at a neutral pH.

The nucleic acid sequence can encode a site-directed mutation at residue E62, D101, or both residues E62 and D101 on the BSMV-CP. In certain embodiments, the site-directed mutation at residue E62, D101, or both residues E62 and D101 on the BSMV-CP is independently selected from the group consisting of glutamine, asparagine, arginine, and lysine.

In certain embodiments, the nucleic acid sequence further comprises a site-directed mutation at a C-terminus of the BSMV-CP encoding a natural amino acid residue for display on a surface of the resulting BSMV VLP. The natural amino acid residue can comprise threonine, serine, glutamate, aspartate, lysine, cysteine, glutamic acid, tyrosine, and/or proline. The natural amino acid residue can comprise comprises a lysine. The natural amino acid can comprise lysine and cysteine.

In certain embodiments, the nucleic acid sequence comprises a site-directed mutation at a C-terminus of the BSMV-CP encoding a SpyTag peptide for display on a surface of the resulting BSMV VLP.

The method can further comprise functionalizing the surface of the BSMV VLP with one or more ligands. Functionalizing can be performed using any method known in the art including, for example, those described herein such as via stimuli-responsive surface functionalization, indirect chemical approaches, indirect bioconjugation, direct chemical conjugation and/or direct protein fusion. It will be appreciated that the natural amino acid and/or SpyTag displayed on the surface of the BSVM VLP enables functionalization with a vast array of ligands.

In certain embodiments, expressing the nucleic acid sequence further comprises constructing a plasmid or an expression vector comprising the nucleic acid sequence; and transforming the plasmid or expression vector into the host. The host can be E. coli and the step of expressing the nucleic acid sequence can be performed at a pH at or between 4-9. In certain embodiments, the step of expressing the nucleic acid sequence is performed at an acidic pH (e.g. a pH of less than about 7). In certain embodiments, the step of expressing the nucleic acid sequence is performed at an alkaline pH (e.g., a pH of greater than about 7).

The BSMV-CP can comprise a BSMV-CP fused with a linker region and the at least one site-directed mutation at residue E62, D101, or both residues E62 and D101 on the BSMV-CP. In certain embodiments, the method can further comprise selecting a length of the linker region based on a desired length in the resulting BSMV VLPs. In certain embodiments, the method further comprises synthesizing one or more nanoparticles using the resulting VLPs.

### EXAMPLES

The following examples serve to illustrate the present disclosure. The examples are not intended to limit the scope of the claimed invention in any way.

### Example 1

### Cloning of BSMV-CP Expression Plasmid

BSMV-CP plasmids with and without TMV-OAS that were codon optimized for bacterial expression were designed pursuant to the present disclosure (shown in subpart A of FIG. 4). More specifically, a codon-optimized DNA sequence encoding BSMV-CP, a linker region, and an OAS (BSMV-CP-linker-OAS; SEQ ID NO: 15) was ordered from IDT (Coralville, IA) and cloned to pET21-1cys-tmv-cp vector (provided by Professor Culver, University of Maryland, College Park), the original plasmid for which was pET-21a(+), with Ndel and XhoI, generating pET21-BSMV-CP-linker-OAS (SEQ ID NO: 26). pET21-BSMV-CP-linker-OAS was subsequently digested with SalI and XhoI to remove the OAS, blunt-ended with Klenow fragment (NEB, Ipswich, MA. Cat. No.: M0210S), ligated back to the backbone itself, generating pET21-BSMV-CP-linker. All strains and plasmids used are listed in Table 2. The BSMV capsid protein sequence was obtained from UniProt (PDB: 517a).

TABLE 2
Strains and plasmids

| | | | | |
|---|---|---|---|---|
| | Relevant | Vector | Plasmid | |
| Name | genotype | backbone | origin | Source |

Strains

| | | |
|---|---|---|
| BL21- | E. coli B F− ompT | Agilent |
| CodonPlus(DE3)- | hsdS(rB− mB−) | Tech- |
| RIPL strain | dcm+ Tetr gal | nologies |
| | λ(DE3) endA Hte | |
| | [argU proL Camr] | |
| | [argU ileY | |
| | leuW Strep/Spec | |
| | resistant | |

Plasmids

| | | | | |
|---|---|---|---|---|
| pET21-BSMV- | bla | pET21- | pBR322 | This |
| CP-linker | | 1cys-tmv-cp | | study |
| | | (original | | |
| | | backbone: | | |
| | | pET- | | |
| | | 21a(+))⁴¹ | | |

Further, for making mutants (E37Q, E37R, E62Q, D68N, D70N, and D101N) to have increased stability, pET21-BSMV-CP-linker-OAS was used as a template for site-directed mutagenesis (E37Q—SEQ ID NO: 20, E37R—SEQ ID NO: 21, E62Q—SEQ ID NO: 23, D68N—SEQ ID NO: 17, D70N—SEQ ID NO: 16, and D101N—SEQ ID NO: 22). All plasmids in the pET-21 vector are ampicillin resistant, with the applicable primers listed in Table 3.

TABLE 3
Primers used for making BSMV-coat protein expression plasmid

| Primer | | | |
|---|---|---|---|
| Name | Sequence (5′>3′) | Template | Expression plasmids |
| E37Q 5′ | SEQ ID NO: 27 | pET21-BSMV-CP | pET21-BSMV-E37Q |
| E37Q 3′ | SEQ ID NO: 28 | | |
| E37R 5′ | SEQ ID NO: 29 | pET21-BSMV-CP | pET21-BSMV-E37R |
| E37R 3′ | SEQ ID NO: 30 | | |
| E62Q 5′ | SEQ ID NO: 31 | pET21-BSMV-CP | pET21-BSMV-E62Q, |
| E62Q 3′ | SEQ ID NO: 32 | | pET21-BSMV-E62Q/D101N |
| D68N 5′ | SEQ ID NO: 33 | pET21-BSMV-CP | pET21-BSMV-D68N |
| D68N 3′ | SEQ ID NO: 34 | | |
| D70N 5′ | SEQ ID NO: 35 | pET21-BSMV-CP | pET21-BSMV-D70N |
| D70N 3′ | SEQ ID NO: 36 | | |
| D101N 5′ | SEQ ID NO: 37 | pET21-BSMV-CP | pET21-BSMV-D101N, |
| D101N 3′ | SEQ ID NO: 38 | | pET21-BSMV-E62Q/D101N |
| D101K 5′ | SEQ ID NO: 39 | pET21-BSMV-CP | pET21-BSMV-D101K |
| D101K 3′ | SEQ ID NO: 40 | | |
| D101R 5′ | SEQ ID NO: 41 | pET21-BSMV-CP | pET21-BSMV-D101R |
| D101R 3′ | SEQ ID NO: 42 | | |
| 199K 5′ | SEQ ID NO: 43 | pET21-BSMV-CP- | pET21-BSMV-D199K |
| 199K 3′* | SEQ ID NO: 44 | D101R | |

*M and N designate random A/C and A/T/G/C bases, respectively.

### Example 2

### BSMV-CP Expression

As discussed in detail herein, VLPs self-assemble to due to interactions between RNA and capsid proteins, and interactions between adjacent capsid proteins. BSMV-CP plasmids with and without TMV-OAS that were codon optimized for bacterial expression were expressed at 37° C. for 4 hours in E. coli before lysing.

More specifically, each BSMV-CP expression plasmid of Example 1 was transformed into E. coli BL21-CodonPlus (DE3), streaked on plates and incubated for 16-20 hours at 37° C. Single colonies were inoculated into Luria-Bertani (LB) broth and grown at 37° C. for 16-20 hours with shaking. The overnight liquid cultures were then diluted a hundred-fold in LB broth and grown until OD600=0.5 before induction with 0.1 mM isopropyl β-D-1-thiogalactopyranoside (IPTG) to express BSMV capsid protein. All BL21-CodonPlus (DE3) liquid cultures or plates containing ampicillin (100 μg/ml) and chloramphenicol (25 μg/ml). The culture was then incubated for 16-20 hours at room temperature (23° C.). Cultures were then centrifuged at room temperature (23° C.) for 5 minutes at 6000 rpm. The supernatant was discarded, and the cell pellet was used directly for purification or stored at −80° C. for processing later.

### Example 3

### BSMV-VLP Purification and Imaging

Crude protein lysate from Example 2 was centrifuged through several rounds to isolate any synthesized VLPs, which were characterized by TEM.

More specifically, BSMV-VLPs were isolated from E. coli transformed with the described plasmids and grown at room temperature for 16-20 hours. The cells were homogenized in 2.5 or 6 ml Bugbuster® protein isolation solution (MilliporeSigma, Burlington, MA. Cat. No: 70584) for 10 minutes at room temperature after which 7.5 μl dithiothreitol was added to 2.5 μl of Lysonase Bioprocessing Reagent Reagent (MilliporeSigma, Burlington, MA. Cat. No: 70584) was added per manufacturer's protocol. The homogenate was further incubated for 10 minutes, then centrifuged at 5,000×g for 10 minutes to remove insoluble cellular debris. The supernatant was fractionated through a linear gradient of Sucrose (MilliporeSigma, Burlington, MA. Cat. No: 70584) spun at 19,000×g for 10 minutes and the top light-scattering band containing the VLPs was collected and further purified by centrifugation at 64,000×g at 4° C. For the sample without doing a gradient, the supernatant was further purified by centrifugation at 10,000×g for 20 minutes at 4° C. The final pellet was suspended in 0.01 M Tris buffer of pH 7.

In preparation for imaging, a 1.5 μl droplet of the VLP suspension was deposited onto carbon formvar copper grids and was negatively stained by a 1.5 μl droplet of phosphotungstic acid (PTA). Images were taken using a 200 kV Tecnai T20 transmission electron microscope (TEM).

In this iteration, TEM images did not show any BSMV rod-shaped VLPs or disk structures (data not shown) suggesting that BSMV CPs were not produced, they failed to self-assemble, or that the isolation procedure was sufficient to capture any produced VLPs. TMV constructs were expressed and purified as positive control and subsequent electron microscopy displayed the presence of TMV VLPs excluding the possibility that the isolation procedure was insufficient. If wildtype CPs are expressed in the host, they form disk-shaped structures. The absence of BSMV disk structures suggests poor soluble CP expression, not necessarily failure to self-assemble.

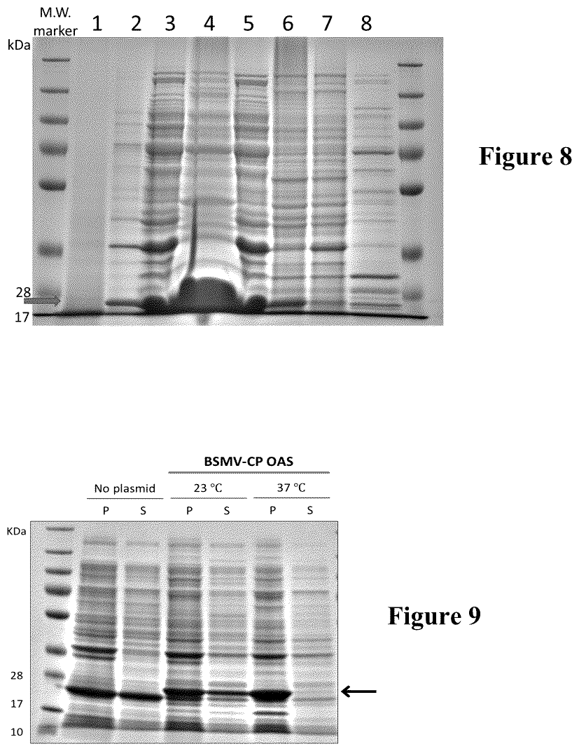

To examine expression, crude protein lysates from E. coli with induced CP plasmids as described above were analyzed via SDS-PAGE. As shown in FIG. 8, SDS-PAGE analysis revealed a relatively heavy band of BSMV protein in the bacterial insoluble pellet suggesting that the majority of the capsid proteins were misfolded.

Because protein aggregation can occur due to the rapid expression and misfolding of proteins at high temperatures, these results support the E. coli host is not able to express soluble protein where the protein of interest is from a host living at 37° C. Instead, the results support that the higher temperature leads to the formation of misfolded insoluble proteins and inclusion bodies composed of insoluble protein aggregates. Given that temperature of this plant virus native host in plants is lower than 37° C. (25-28° C.), the BSMV-CP folding appears to be thermodynamically unfavorable at the higher temperature (37° C.).

### Example 4

### Size Measurement by Dynamic Light Scattering

Alternative size characterization studies were also performed, with the produced VLPs subjected to dynamic light scattering (DLS). The refractive index of purified BSMV-VLPs was detected with 1.3351 by refractometer and the size of BSMV-VLPs was measured with dynamic light scattering by Malvern Zetasizer Nano ZS (Malvern Panalytical Ltd, United Kingdom). The refractive index and viscosity of the Tris resuspension buffer were 1.35 and 1.00037⁴², respectively.

DLS revealed a bimodal distribution with peaks at 476.5 nm (92.3%) (which corresponds with self-assembled VLPs) and incomplete disks at 37.99 nm (7.7%) (see Table 4). Accordingly, the majority of assembled CPs formed complete VLPs that were variable in length. As hydrodynamic radii are inherently larger than the actual size detected by TEM, DLS provides an alternative way to rapidly check the VLPs quality rather than an absolute metric of size.

TABLE 4
Hydrodynamic radii obtained by dynamic light scattering (DLS).

| Size | | Standard deviation | Coefficient |
|---|---|---|---|
| (spherical diameter) | Intensity | (diameter) | of variation |
| 476.5 nm | 92.3% | 157.7 nm | 33.10% |
| 37.99 nm | 7.7% | 6.412 nm | 16.88% |

While spherical diameter is listed in Table 4, it should be noted that the measuring instrument views each rod-shaped VLP as a spherical due to the multiple perspective angles from which measurements are taken. While the VLPs are rod-shaped instead of spherical, the resulting diameter information correlates with the accurate VLP diameter obtained from the TEM.

### Example 5

### Self-Assembly and Optimization

As discussed in detail herein, VLPs self-assemble due to interactions between RNA and capsid proteins, and interactions between adjacent capsid proteins. In furtherance of investigations into the occurrence of self-assembly, the expression temperature was reduced to 23° C. and the expression time extended from 4 hours to 16 hours to slow the protein expression rate and enable proper protein folding. Subsequent SDS-PAGE analysis revealed a significant increase in soluble capsid protein see FIG. 9). Subsequent isolation of potential particles and characterization, electron microscopy images demonstrated the presence of self-assembled rod-shaped BSMV VLPs assembly (see subpart C of FIG. 4). This data supports that TMV-OAS was recognized by BSMV-CP and functions to initiate the VLP self-assembly, while only the disks structure formed in the BSMV-CP absent of OAS (see subpart B of FIG. 4). Furthermore, these results support that poor protein expression can be improved by adjusting the induction temperature to increase the yield of soluble proteins. Importantly, this is the first time that BSMV-VLPs have been expressed from a microbial-based protein expression system at neutral pH and observed by TEM.

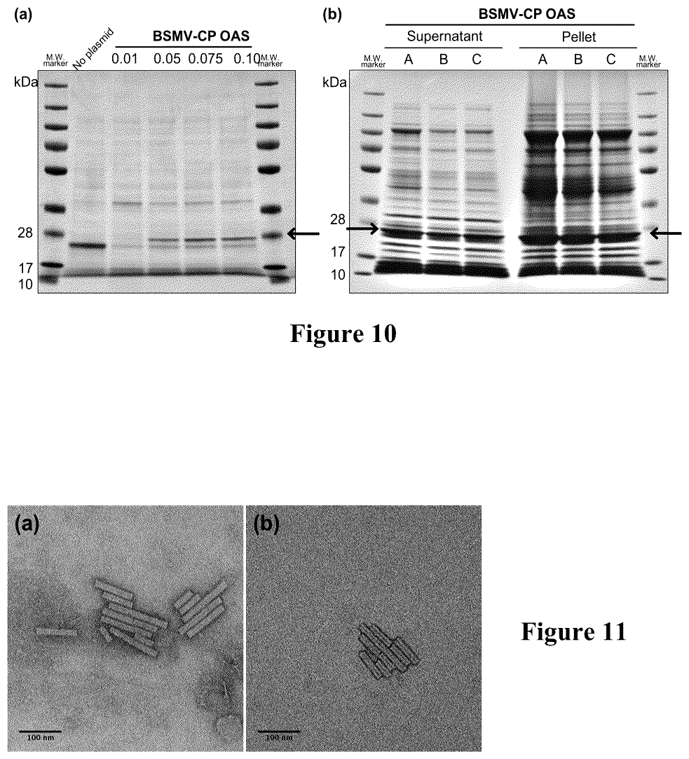

Following the favorable results indicating VLP self-assembly in E. coli, studies were conducted to optimize the expression and purification process. Different concentrations of IPTG inducer from 0.10 mM, 0.075 mM, 0.050 mM to 0.010 mM were tested with respect to induction of expression of BSMV-CPs. There, the expression levels of BSMV-CPs in the soluble fraction was monitored by SDS-PAGE. As shown in subpart (a) of FIG. 10, the results support that 0.075 and 0.1 mM IPTG concentrations produced higher yield of soluble CP as compared to the lower concentrations tested. The effect of the addition of resuspension buffer in solubilizing a final pellet was also determined to enhance the yield of soluble fraction. Sodium phosphate buffer is usually used in protein solubilization but it caused precipitate over time. Water and tris(hydroxymethyl)aminomethane (Tris-HCl) were then tested as final resuspension buffers. Subpart (b) of FIG. 10 supports that both water and Tris buffer prevent precipitation of VLPs. Furthermore, the results support that Tris buffer was superior as it generally stabilized more BSMV protein and gave rise to less cellular proteins.

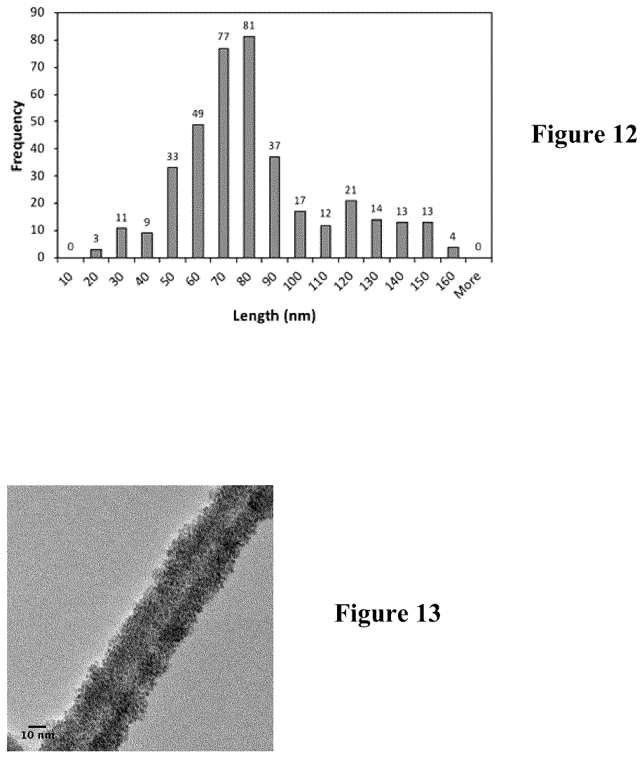

Based on the optimization parameters identified, the cells were further purified with sucrose cushion centrifugation (a technique utilized for purification without resulting in a firm pellet). As shown in FIG. 11, electron microscopy displayed the presence of BSMV-VLPs in both top and bottom layers of the sucrose cushion with no significant differences in length or size. The purified BSMV-VLPs yielded variable structure between 20-160 nm length. The majority of the expressed nanotubes were found to be between 70 nm to 90 nm long. (see FIG. 12).

### Example 6

### Metal Coating

To examine the capability as biotemplates of the bacteria assembled BSMV-VLPs, the VLPs were coated with palladium via hydrothermal synthesis in the absence of extra reducing agent and incubated in a reaction vessel with Na₂PdCl₄ precursor solution.

Metal coating on VLPs was performed in a 100 ml CSTR reactor vessel at a controlled temperature of 57° C. As in a typical nanoparticle synthesis, the precursor sodium tetrachloropalladate (II) (Na₂PdCl₄) (98%, Sigma Aldrich, St Louis, MO) aqueous solution (concentration is usually between 0.3 mM and 6 mM) was added into the vessel containing the purified VLPs after heating to the desired reaction temperature. 0.3 mL aliquots of the solution were collected regularly during the course of the reaction for ex-situ study by UV-vis characterization. The solution was immediately placed on ice to quench the reaction for the absorbance measurement by UV-Vis spectrophotometer (Varian Cary 100) at 25° C. Poly(methyl methacrylate) (PMMA) plastic cuvettes (VWR Scientific Prod Midwest, Radnor, PA) were used for the UV-vis characterization. The nanoparticles were washed repeatedly to remove residual salt and precursor solution by redispersing them in water. Thicker coatings were achieved by reincubating the washed nanorods in Na₂PdCl₄ solution and recoated multiple times. Millipore water was used in all experiments.

FIG. 13 supports that the BSMV-VLPs were fully coated with a uniform layer of palladium nanoparticles. Accordingly, not only were the BSMV-VLPs able to drive surface-mediated mineralization with palladium, but also retained their robust stability during biomineralization reaction.

### Example 7

### BSMV Protein Crystal Structure Alignment

There are various approaches to stabilize rod-shaped plant viruses such as TMV and BSMV, whose structures were recently resolved. For example, cysteine insertions enabled disulfide bond formation between coat proteins, yielding stable TMV nanorods up to pH 11. While these made efficient catalysts for alkaline hydrogen evolution, disruption by reducing agents can restrict applicability to electroless metal deposition and intracellular applications. Therefore, BSMV was stabilized via CCC engineering.

Analogous CCC candidate residues to those validated in TMV were identified via sequence structural alignment. The capsid proteins of TMV (PDB: 2xea) and BSMV (PDB: 5a7a) were structurally aligned via TM-align (version 20190822), a publicly available algorithm for sequence independent protein structure comparison, using the default setting. Zhang & Skolnick, TM-align: a protein structure alignment algorithm based on the TM-score, Nucleic Acids Research 33: 2302-2309 (2005). TMV-validated CCC residues E50 and D77 were used to find the corresponding residues on BSMV capsid protein based on the structural proximity of negatively-charged residues. The individual and overlay structures were downloaded from TM-align output and visualized via pymol.

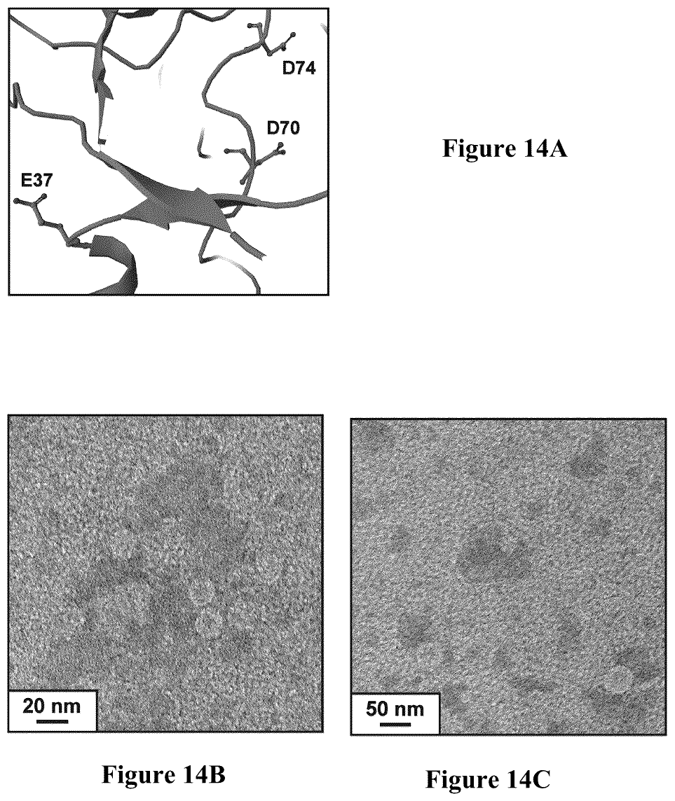

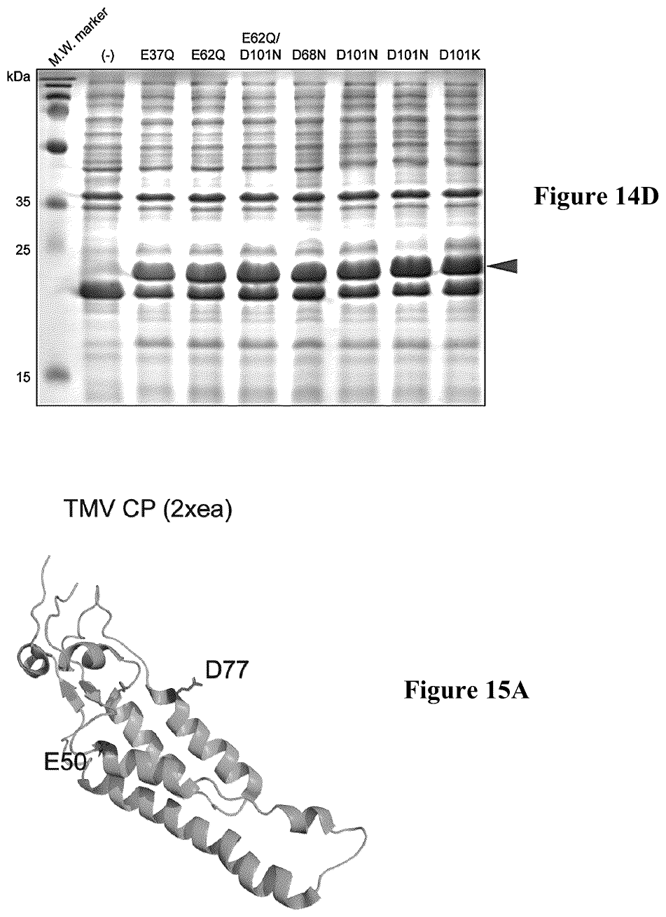

Crystallization and structural elucidation of chimeric BSMV VLPs revealed close axial contacts between the carboxylate residues on adjacent capsid proteins located at Glu37 (E37), Asp70 (D70), and D74 that were proposed as the CCC (FIG. 14A). E37 has been hypothesized to interact with both D70 and D74 on adjacent capsid proteins; thus, point mutations of E37 to a neutral (Gln/Q) or positive (Arg/R) residue was thought to be potentially sufficient to neutralize the CCC repulsion and stabilize VLPs at alkaline pH. The putative candidate residues were then subjected to site-directed mutagenesis for experimental validation.

### Example 8

### Cloning of BSMV Capsid Protein Mutants

To evaluate their role in assembly, we expressed transcripts encoding BSMV capsid protein point mutants in E. coli. E. coli strains and plasmids used in this study are listed in Table 5.

TABLE 5
Strains and plasmids

| | | Vector | Plasmid | |
|---|---|---|---|---|
| Name | Relevant genotype | backbone | origin | Source |
| Strain | E. coli BF⁻ ompT hsdS | N/A | N/A | Agilent |
| BL21- | (r_{B}m_{B}) dcm⁺ | | | Technologies |
| CodonPlus | Tet^{r} gal λ(DE3) | | | |
| (DE3)-RIPL | endA The [argU proL Camr] | | | |
| | [argU ileY leuW Strep/Spec^{r} | | | |
| Plasmids | | | | |
| pET21- | BSMV-CP-linker-OAS, bla | pET21-1cys- | pBR322 | Lee et al. |
| BSMV-CP | | tmv-CP | | (2020), |
| | | | | supra. |
| pET21- | BSMV-CP-E37Q-linker, | pET21-BSMV- | pBR322 | Present study |
| BSMV-CP- | bla | CP | | |
| E37Q | | | | |
| pET21- | BSMV-CP-E37R-linker, | pET21-BSMV- | pBR322 | Present study |
| BSMV-CP- | bla | CP | | |
| E37R | | | | |
| pET21- | BSMV-CP-E62Q-linker, | pET21-BSMV- | pBR322 | Present study |
| BSMV-CP- | bla | CP | | |
| E62Q | | | | |
| pET21- | BSMV-CP-D68N-linker, | pET21-BSMV- | pBR322 | Present study |
| BSMV-CP- | bla | CP | | |
| D68N | | | | |
| pET21- | BSMV-CP-D70N-linker, | pET21-BSMV- | pBR322 | Present study |
| BSMV-CP- | bla | CP | | |
| D70N | | | | |
| pET21- | BSMV-CP-D101N-linker, | pET21-BSMV- | pBR322 | Present study |
| BSMV-CP- | bla | CP | | |
| D101N | | | | |
| pET21- | BSMV-CP-D101R-linker, | pET21-BSMV- | pBR322 | Present study |
| BSMV-CP- | bla | CP | | |
| D101R | | | | |
| pET21- | BSMV-CP-D101K-linker, | pET21-BSMV- | pBR322 | Present study |
| BSMV-CP- | bla | CP | | |
| D101K | | | | |
| pET21- | BSMV-CP- E62Q/D101N- | pET21-BSMV- | pBR322 | Present study |
| BSMV-CP- | linker, bla | CP | | |
| E62Q/D101N | | | | |
| pET21- | BSMV-CP-E62Q/ 199K- | pET21-BSMV- | pBR322 | Present study |
| BSMV-CP- | linker, bla | CP | | |
| D101R/199K | | | | |

All molecular biology manipulations were carried out according to standard practices known in published literature. See, e.g., Sambrook et al., Molecular Cloning: a Laboratory Manual, Cold Spring Harbor Laboratory Press, 1989. B SMV CP CCC-mutants were made via site-directed mutagenesis. Braman et al., Site-directed mutagenesis using double-stranded plasmid DNA templates, in: M. K. Trower (Ed.), In Vitro Mutagenesis Protocols, Humana Press, Totowa, NJ, pp. 31-44 (1996).

Briefly, plasmid pET21-BSMV-CP (see Lee et al. (2020), supra) was amplified with Phusion DNA polymerase (Thermo Fisher Scientific, Waltham, MA. Cat. No: F530S) and the mutagenic primers described in Table 3 above.

Cleaned up reaction products were then digested with DpnI to remove unmutated plasmid before transformation into E. coli.

The BSMV-capsid protein expression plasmids were transformed into E. coli BL21-CodonPlus (DE3)-RIPL (Agilent Technologies, Santa Clara, CA. Cat. No.: #230280). The bacteria were streaked onto plates containing LB media plus 100 μg/ml ampicillin and 25 μg/ml chloramphenicol and incubated for 16-20 h at 37° C. Single colonies were selected, inoculated into LB broth, and incubated at 37° C. for 16-20 hours at 250 RPM. The liquid cultures were then diluted a hundred-fold in LB broth and incubated at 37° C. until an OD600 of 0.5. The cultures were induced with the addition of 0.1 mM isopropyl β-D-1-thiogalactopyranoside (IPTG) to express the BSMV capsid protein followed by incubation for 16 hours at room temperature (˜23° C.) to express capsid protein.

All BL21-CodonPlus (DE3)-RIPL liquid cultures or plates contained ampicillin (100 g/ml) and chloramphenicol (25 μg/ml). Bacteria were collected by centrifugation at room temperature for 5 minutes at 6000 rpm. The pellet containing the bacteria was used directly for isolation of BSMV VLPs or stored at −80° C.

Protein expression and purification for BSMV carboxylate residue mutants were performed as described previously. See Lee et al. (2020), supra. In brief, BSMV VLPs were isolated from E. coli cell pellets by resuspension in BugBuster® Protein Extraction Reagent (MilliporeSigma, Burlington, MA) supplemented with Lysonase™ Bioprocessing Reagent (MilliporeSigma, Burlington, MA) per the manufacturer protocol. Lysates were then incubated at room temperature for 10 minutes and centrifuged at 19,000 g for 10 minutes to remove the insoluble lysates. VLPs were isolated from the soluble protein lysates by centrifugation at 64,000 g at 4° C. for 1 hour. The isolated VLP pellets were resuspended in 10 mM Tris-HCl at pH 7 or pH 9 at 4° C.

To validate coat protein expression, cell lysates were analyzed on 14% polyacrylamide gels (Thermo Fisher Scientific, Waltham, MA. Cat. No.: XP04200BOX). 14 μl of protein lysate was mixed with an equal volume of 2× Tris-glycine SDS Sample Buffer (Thermo Fisher Scientific, Waltham, MA. Cat. No.: LC2676) and supplemented with 2 μl of 1 M dithiothreitol (DTT) (Thermo Fisher Scientific, Waltham, MA. Cat. No.: AC426380100). Samples were then incubated at 85° C. for 5 minutes to denature the proteins. Samples were then placed on ice for 5 minutes before electrophoresis. PageRuler™ Plus Prestained Protein Ladder (Thermo Fisher Scientific, Waltham, MA. Cat. No.: 26620) was used as a molecular weight standard.

The gels were run at 120 V for an hour before staining with Coomassie blue (Fisher Scientific, Pittsburgh, PA. Cat. No.: BP101-25) for 10 minutes. Gels were then destained with destaining buffer (10% glacial acetic acid and 10% methanol) overnight before visualization under visible light with an Azure c400 imager (Azure Biosystems, Dublin, CA).

TEM samples were prepared for imaging by placing 1.5 μl of the VLP suspension onto formvar/carbon-coated copper grids followed by an equal amount of ACS-grade phosphotungstic acid (PTA, stock concentration: 1%) for negative staining. After 15 seconds, the excess liquid was wicked from the grid with 3MM paper, and the grid was allowed to dry. At least 50 images were taken per sample using Tecnai T20 transmission electron microscope (200 kV). More than 20 images with good contrast and focus were analyzed with ImageJ software to measure the dimensions of over 180 nanorods. Dynamic light scattering was performed with a Malvern Zetasizer Nano ZS instrument. Following 18 days of incubation, 0.40 ml of each sample was placed in triple-rinsed ZEN0040 cuvettes equilibrated to 25° C. for 30 seconds before each measurement. The data shown is the average of 10 readings, which were taken for 10 seconds each at a 173° backscatter measurement angle. Size distributions were obtained with a general purpose analysis model. All measurements showed good second-order correlation functions with a y-intercept between ˜0.9 and 1.

SDS-PAGE analysis confirmed successful heterologous expression of mutant BSMV CPs (FIG. 14D). The neutral (E37Q) and positive (E37R) mutants did not result in the formation of VLP rods (FIGS. 14B and 14C). Analogous single point mutations in TMV (e.g., E50Q) resulted in VLP rods and reduced disassembly at elevated pH. See Culver et al., Site-directed mutagenesis confirms the involvement of carboxylate groups in the disassembly of tobacco mosaic virus, Virology 206: 724-730 (1995). These results suggest that E37 is not an amenable site to neutralize repulsive CCC interactions.

The suggested CCC residues (E37, D70, and D74) have been shown to be very close to the salt bridge formed between D44 and R69. Point mutations of residue E37 may have disrupted the salt bridge, thus preventing self-assembly.

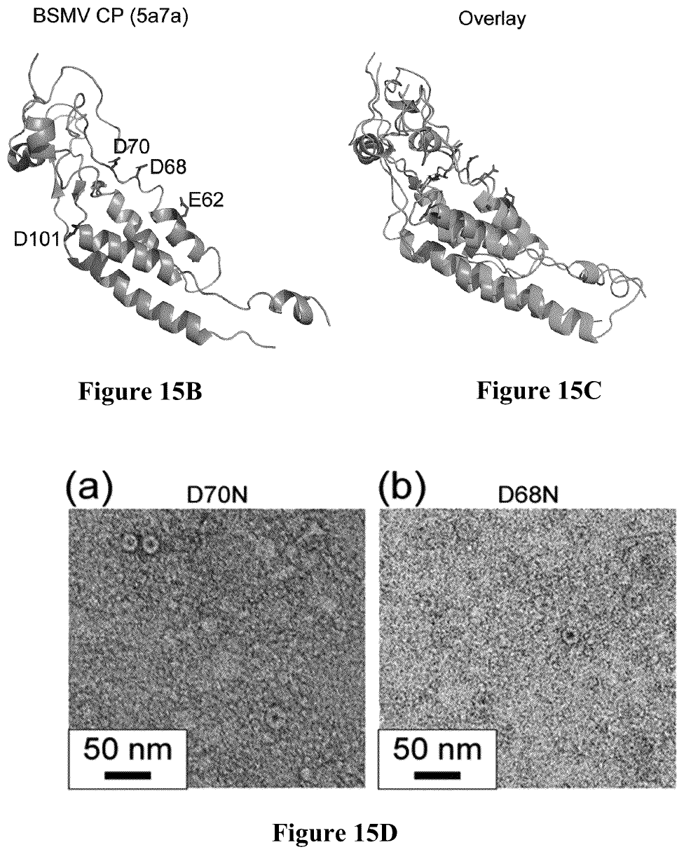

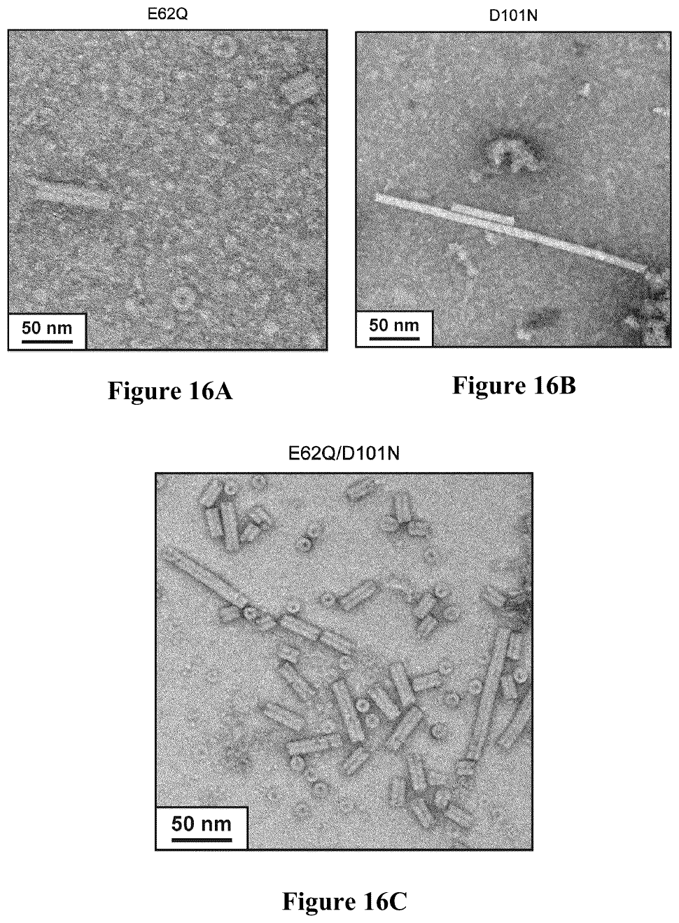

Structural analyses did not identify alternate interacting residues for the rest of the putative CCC (D70, D74). Thus, an alternate structure-guided approach was pursued based on the structural similarities between TMV and BSMV capsid proteins. Superposition of BSMV and TMV capsid protein crystal structure revealed that D70 and three novel residues (E62, D68, and D101) closely aligned with established TMV CCC residues (FIGS. 15A-15C). D101 of BSMV corresponded to E50 of TMV, while E62, D68, or D70 of BSMV was analogous to D77 of TMV (FIGS. 15A and 15B). To evaluate this new CCC as a candidate for VLP stabilization, the negative aspartate (D) was mutated to neutral asparagine (N) or positive lysine (K), and negative glutamate (E) to neutral glutamine (Q) or positive arginine (R) through site-directed mutagenesis. Neither D68N nor D70N resulted in the formation of nanorod-shaped VLP (FIG. 15D). In contrast, E62Q and D101N successfully led to clear, rod-shaped capsid protein assembly (FIGS. 16A and 16B). Thus, E62 and D101 formed a previously unrecognized BSMV CCC that tolerated site-directed mutagenesis for potential stabilization.

Combining these mutations also resulted in successful VLP assembly (FIG. 16C). These results validated the role of E62 and D101 contacts in BSMV self-assembly and demonstrated that these protein: protein interactions can be engineered to stabilize assembly of BSMV VLPs.

Accordingly, to test if replacing the repulsive negative CCC interactions with attractive positive/negative interactions will have a strong stabilizing effect on particle assembly, D101 was mutated with positively charged residues such as arginine or lysine that can attract the negative charge of the opposing glutamate (to result in D101K (includes lysine) and D101R (includes arginine)).

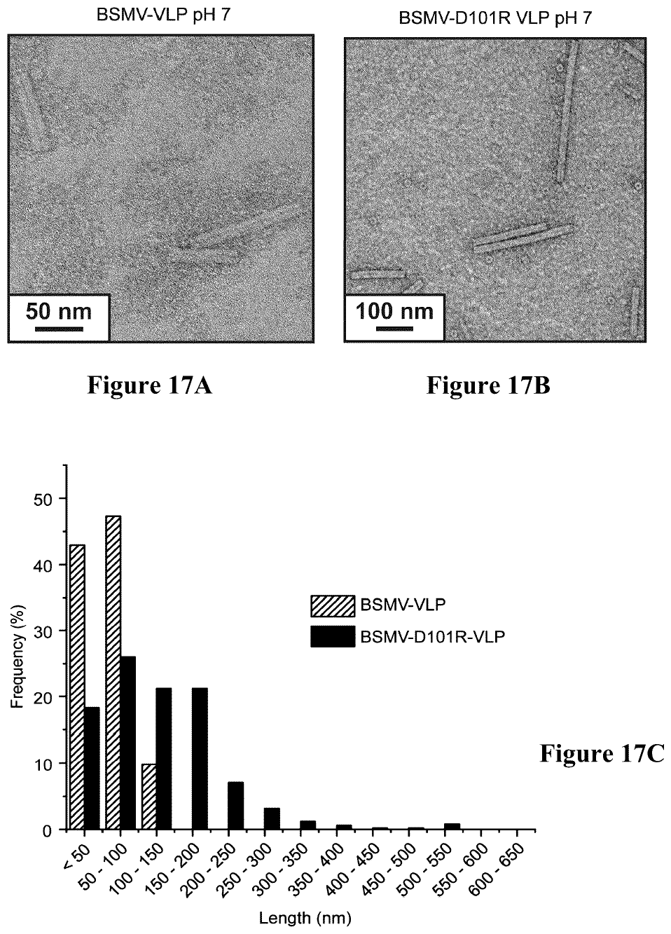

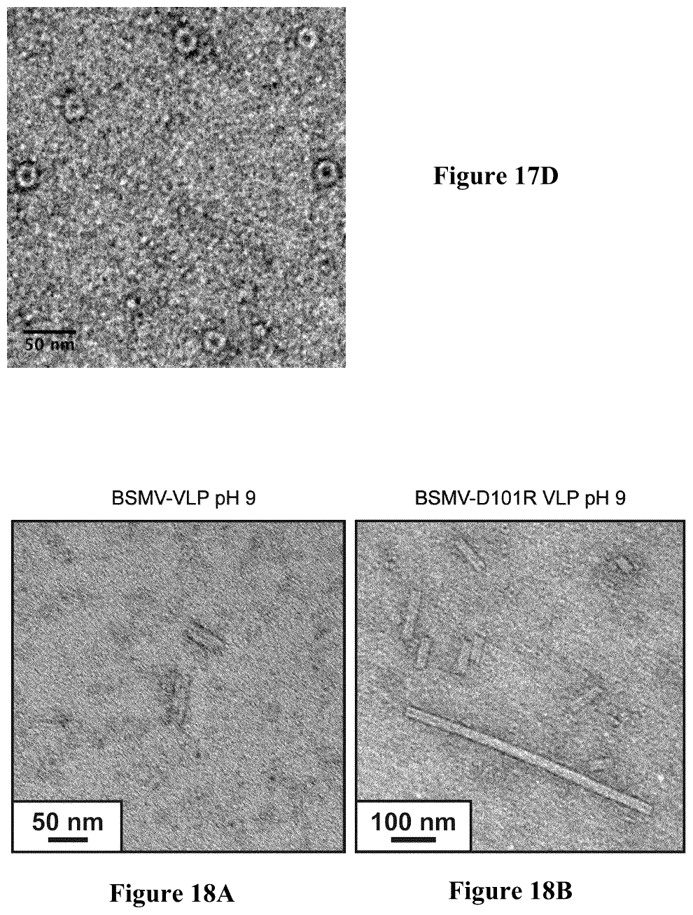

Although D101K did not result in observable VLPs (FIG. 17D)), D101R mutants clearly formed viable VLP rods (FIGS. 17A-17C). The assembly of D101R mutants into VLPs was attributed to the geometric effects of residues. Arginine contains a guanidinium group that allows formation of three salt-bridge and hydrogen bonds, whereas the amino group in lysine allows only one such interaction. Stronger electrostatic interactions and improved stability in proteins generated by arginine have been demonstrated indirectly by illustrating higher number of salt-bridges in the presence of arginine than lysine. These results support that residue geometry and position, and not only charge, are critical for stabilizing interactions between the capsid protein subunits.

### Example 9

### Assessment of BSMV CCC Mutant Stability Under Varying Conditions

BSMV-D101R VLPs were expected to be the most stable mutants generated due to the stronger electrostatic attraction between oppositely charged residues than charge-neutral interactions. Zhou & Pang, Electrostatic interactions in protein structure, folding, binding, and condensation, Chemistry Reviews 118: 1691-1741 (2018). As such, BSMV-wildtype and BSMV-D101R VLPs were analyzed under neutral and alkaline conditions to test for stabilized self-assembly.

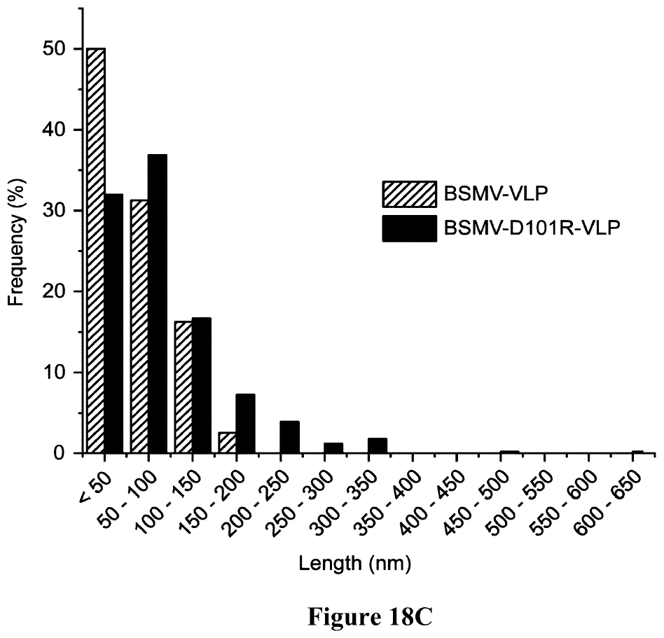

BSMV-VLP pellets were isolated and resuspended by continuous agitation in 0.1 M Tris-HCl at either pH 7 or 9 for 24 hours, followed by transmission electron microscopy (TEM) for characterization. TEM images indicated the presence of rod-shaped particles for both wildtype and D101R mutants at pH 7 and 9. TMV literature suggests that non-acidic pH leads to partial disassembly of TMV nanorods. The ImageJ analysis of the TEM images was consistent with this expectation, showing a higher average rod length for D101R (125 nm) than wildtype BSMV VLPs (62 nm) at neutral pH (FIGS. 17A-17C). Notably, there was a similar, ˜2-fold, difference in alkaline conditions, with D101R having an average rod length of 91 nm and wildtype BSMV VLPs having an average length of 40 nm (FIGS. 18A-18C). These results were consistent with DLS data.

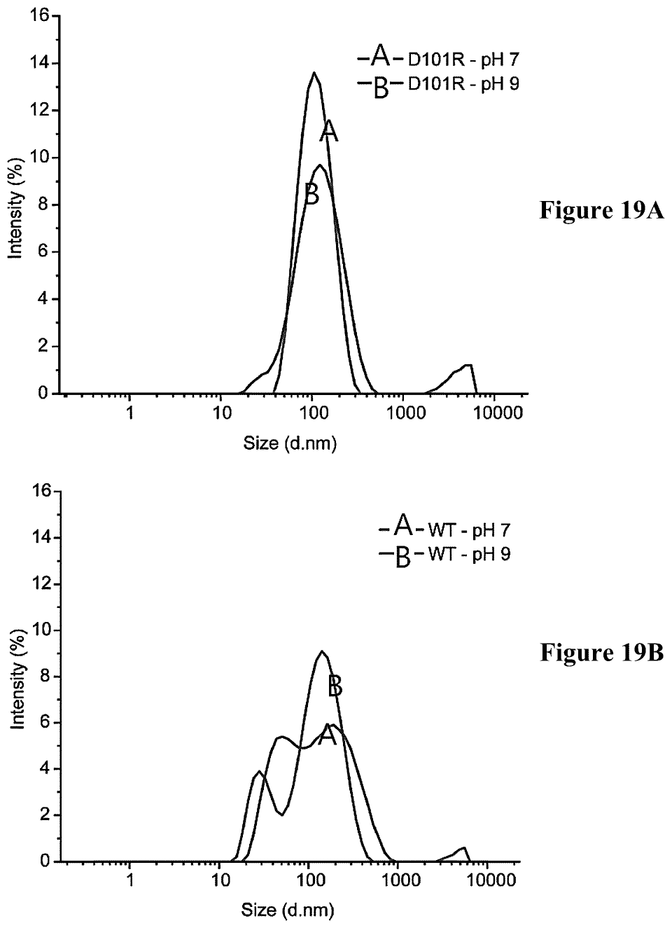

The mutant D101R VLPs did not show a clear shift, although a small tail appears at lower sizes at pH 9 (FIG. 19A). However, wildtype VLPs showed a leftward shift in the size distribution upon alkaline incubation (FIG. 19B). Notably, the wildtype samples showed a distinctly large peak at lower sizes, regardless of pH, indicating that they have a larger fraction of (partially) disassembled rods and/or disks multimers. The longer rods in D101R mutants than wildtype VLPs at both pH 7 and 9 indicated that attractive electrostatic interactions played a major role in stabilizing BSMV-D101R-VLPs. Furthermore, there was a greater range of length and aspect ratio in the D101R mutants, which could be beneficial for alkaline applications such as pancreatic drug delivery. Alkaline stability also favors potential uses in inorganic coating, for example to aid in the development of solar cells, tough nanocomposites, and other technologies that use rod-shaped metal nanoparticle fillers.

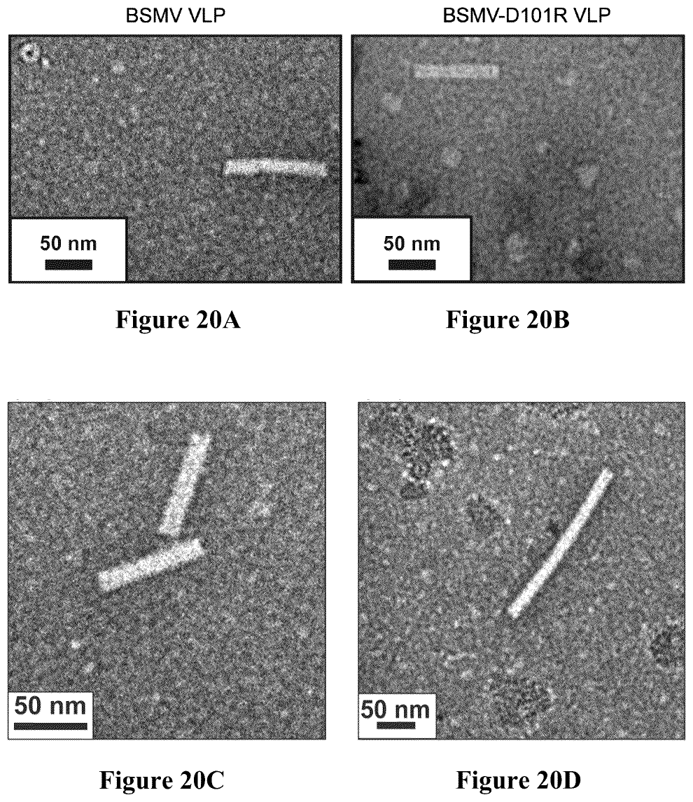

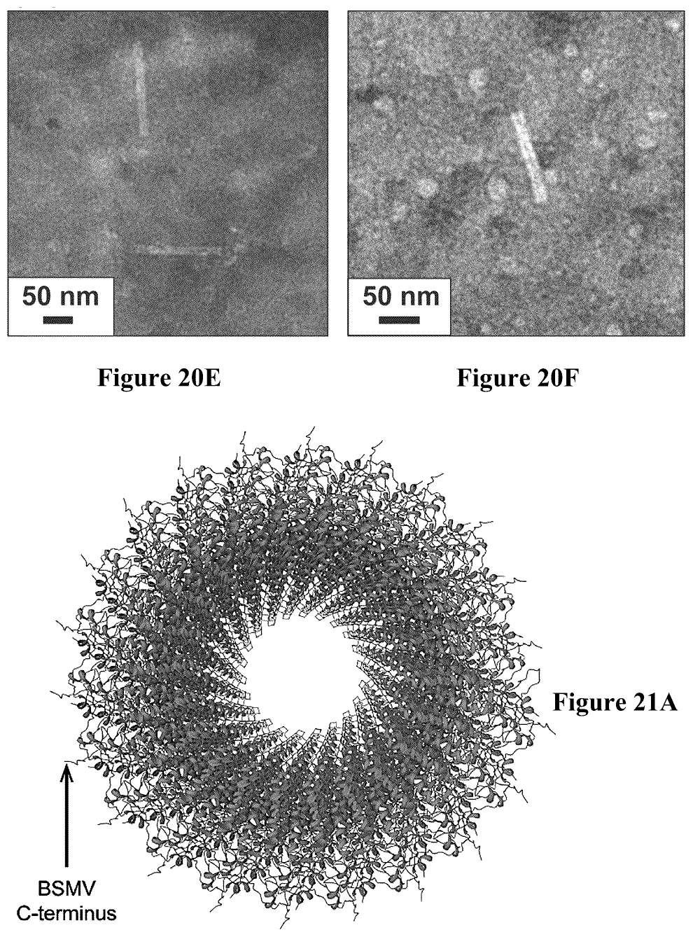

After validating that replacing the CCC with a positively-charged residue had a strong stabilizing effect in neutral and alkaline conditions, we sought to check the assembly state in acidic conditions. Although wildtype VLPs are known to form rod-shaped particles at acidic pH, it was expected that the D101R mutants would be relatively destabilized around pH 4, as the glutamate residue (E62) has a pKa of 4.15; in that case, the interaction residue pair would switch from positive/negative to positive/neutral, leading to reduced attraction. However, surprisingly, TEM images confirmed that BSMV-D101R-VLPs, like the wildtype BSMV VLPs, remained assembled at pH 4 (FIGS. 20B-20F). The positive/neutral charge of the glutamate side chain at pH 4 still resulted in the presence of rods, which mirrored the assembly of wildtype VLPs with neutral/negative interactions at this condition (FIGS. 20A and 20B). These results demonstrate the stability of BSMV-D101R VLPs over a wide range of pHs, thus supporting it is a beneficial platform for further engineering, such as surface functionalization.

### Example 10

### Surface Functionalization of BSMV Mutants

A predictive model of D101R coat protein dimers was computed using AlphaFold 2 (commercially and publicly available software) (FIG. 21B). Jumper et al., Highly accurate protein structure prediction with AlphaFold, Nature 596: 583-589 (2021). Since this open source algorithm could not tolerate the computational cost of modeling the entire VLP, the analysis was restricted to a dimer that resembles coat protein interactions seen in the VLP structure as determined by cryo-EM. The models were analyzed using Swiss PDB Viewer (also known as DeepView) to identify solvent accessible residues for each coat protein in this dimer. Accessible residues were identified by setting accessibility threshold to 50%.

Lys9 (2101 in FIG. 21B) was the only surface accessible lysine residue in one monomer (top), while Lys180 (2105 in FIG. 21B) was the only accessible residue in the other monomeric coat protein (bottom). Neither Lys9 2101 on the top coat protein nor Lys180 2105 on the bottom coat protein were accessible when oligomerized into a VLP. The dimeric structure was consistent with this calculation, as both residues were clearly buried at the protein:protein interface. This supports that a fully assembled VLP, with nearly all proteins contacting lateral and longitudinal neighbors, would not have any surface-exposed lysine residues. However, all three C-terminal residues were surface accessible for both proteins in the dimer (2103 in FIG. 21B.

Accordingly, while the AlphaFold2 prediction of D101R dimer suggested there were no inherently accessible lysine residues in the assembled VLP, it did support that C-terminal insertions were likely exposed on the particle surface. Further, this model supports that the C-terminus of the D101R/199K mutant will be the only lysine residue present.

Cryo-electron microscopy elucidation of BSMV capsid protein and virion structure showed particle surface-exposed C-termini (FIG. 21A), which provides a site to incorporate functional groups for subsequent chemical modification.

To test if the D101R VLP mutant assembly can tolerate single amino acid insertions at the C-terminus, site-directed mutagenesis of the D101R construct was performed to insert a lysine residue at the C-terminus (BSMV-D101R/199 K) and the transcripts were expressed in E. coli.

The D101R mutant with an additional C-terminal lysine insertion was made through PCR from pET21-BSMV-CP-D101R with primers 199 K 5′/199 K 3′; the 3′ primer contained a random codon inserted right before the stop codon and introduced AgeI sites downstream.

pET21-BSMV-CP and insert were then digested with AgeI and XbaI to enable swapping of wildtype BSMV-CP and the BSMV-D101R/199 K insertion mutant via standard recombinant biotechnology approaches. See Braman et al. (1996), supra. Colonies were screened to identify one with a lysine-encoding codon inserted at the C-terminus. All constructs were verified via Sanger sequencing at Genewiz (South Plainfield, NJ).

To obtain pure BSMV-D101R/199 K VLPs for further surface functionalization, the VLPs were prepared as described above, except a different purification protocol was followed. After the VLPs were isolated from E. coli using BugBuster® Protein Extraction Reagent and Lysonase™ Bioprocessing Reagent, the suspension was incubated for 15 minutes at room temperature on a shaker to lyse the cells. The lysis was followed by centrifugation at 21,000 g for 15 min at 4° C. to remove the insoluble lysates. The VLPs in the soluble protein lysates were pelleted by ultracentrifugation on a 25% (w/v) sucrose cushion at 30,000 rpm at 4° C. for 3.5 hours. The pellet was allowed to resuspend in 1×phosphate buffered saline (PBS) at 4° C. for 2 days, after which the suspension was passed through a 0.22 μm syringe filter for future use.

SDS-PAGE analysis showed successful heterologous expression of the BSMV-D101R/199K coat proteins (FIG. 21D). The assembly of the modified D101R/199K capsid proteins into rods was validated by TEM images (FIG. 21C).

### Example 11

### Reaction Kinetics Between Functionalized BSMV-D101R/199K VLPs and Fluorescamine

Owing to the versatile pH stability of BSMV-D101R VLPs, D101R/199 K VLPs were used to study the kinetics of a labeling reaction at acidic, neutral, and alkaline conditions.

BSMV-D101R/199 K VLPs suspended in 0.01 M N-2-hydroxyethylpiperazine-N′-20ethanesulfonic acid (HEPES) buffer at pH 7.2 were divided into three aliquots. Each aliquot was buffer-exchanged thrice with 0.1 M pH 5 citrate buffer, 1×PBS (pH 7), and 0.1 M pH 9 carbonate-bicarbonate buffer, respectively, using Amicon® Ultra centrifugal filter units with 100 kDa molecular weight cut-off (MWCO) (MilliporeSigma, Burlington, MA). Stock solution of fluorescamine (concentration: 3.6 mM) was prepared by dissolving 250 mg of fluorescamine powder (Sigma-Aldrich, St. Louis, MO. Cat. No: F9015-250MG) in 25 ml of anhydrous dimethyl sulfoxide (DMSO) (Fisher Scientific, Pittsburgh, PA. Cat. No.: AC326880010).

VLPs at pH 5, 7, and 9 were mixed with fluorescamine in a 1:100 VLP:fluorescamine molar ratio in a Nunc™ black/clear bottom 96-well plate (Thermo Fisher Scientific, Waltham, MA. Cat. No. 265301). Buffers at corresponding pH were used as blanks. Fluorescence at each time point was measured using a microplate reader (SpectraMax iD5, Molecular Devices, Downingtown, PA) at an excitation wavelength of 380 nm and emission wavelength of 470 nm. Blank subtracted fluorescence intensity values were then plotted against time. Fluorescence measurements were performed in triplicate. Error bars in the graph represent the standard error of mean fluorescence intensities.

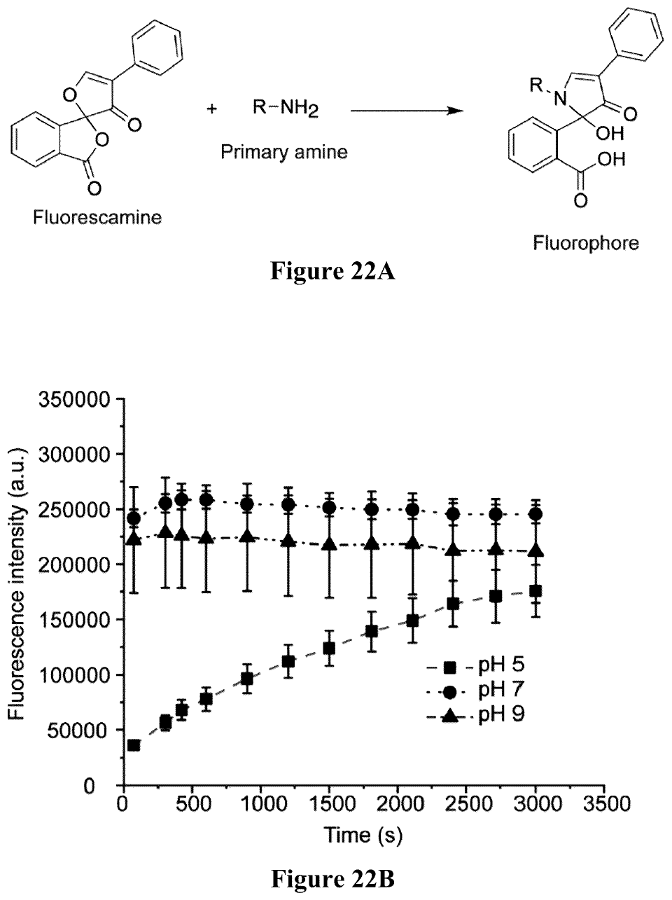

Reaction between surface-exposed lysine residues and fluorescamine (FIG. 22A) showed faster kinetics at neutral and alkaline pH than acidic pH (FIG. 22B), which took over 41 times as long to reach completion. The fluorescence intensity of the resulting fluorophore is known to stay constant between pH 4.5 and 10.5; thus, the measured fluorescence intensity serves as a proxy for the extent of reaction. The trends were as expected; the side chain amines in lysine would be protonated at pH 5, resulting in lower reactivity than VLPs in neutral and alkaline pH. As the unprotonated amines reacted with fluorescamine, the reaction equilibrium shifted towards more amines getting unprotonated, thus validating the increase in fluorescence intensity with time at pH 5. The reaction between fluorescamine and amine groups reached the maximum within 10 minutes for both pH 7 and 9 and matched the pH conditions typically used for conjugation reactions with lysine residues. These results support that BSMV-D101R VLP mutants can serve as a pH-stable platform for surface functionalization, thus providing opportunities for use in numerous applications.

### Example 12

### Functionalization of D101R/199C VLPs

Site-directed mutagenesis of the D101R construct was performed to insert a cysteine residue at the C-terminus (BSMV-D101R/199 K) and the transcripts were expressed in E. coli.

The D101R mutant with an additional C-terminal cysteine insertion was made through PCR from pET21-BSMV-CP-D101R with primers 199 C 5′/199 C 3′; the 3′ primer contained a random codon inserted right before the stop codon and introduced AgeI sites downstream.

pET21-BSMV-CP and insert were then digested with AgeI and XbaI to enable swapping of wildtype BSMV-CP and the BSMV-D101R/199 C insertion mutant via standard recombinant biotechnology approaches. See Braman et al. (1996), supra. Colonies were screened to identify one with a cysteine-encoding codon inserted at the C-terminus. All constructs were verified via Sanger sequencing at Genewiz (South Plainfield, NJ). The BSMV-D101R/199C VLPs were purified as previously described in connection with the BSMV-D101R/199K VLPs.

The BSMV-D101R/199C VLPs suspended in 0.01 M potassium phosphate buffer at pH 7.2 were divided into two aliquots. Stock solution of fluorescein maleimide (concentration: 17 mM) was prepared by dissolving 25 mg of fluorescein maleimide powder (TCI Chemicals, Portland, OR) in 3.4 mL of anhydrous DMSO.

One of the D101R/199C VLP aliquots was incubated with 10-fold molar excess of fluorescein maleimide. The second aliquot was incubated with only DMSO equal to the volume of dye added to the first aliquot, but without the dye as a control. The reaction was allowed to occur for 24 hours at room temperature. The excess dye was removed and the VLPs were analyzed by size exclusion fast protein liquid chromatography using a Superdex 200 10/300 GL size exclusion column on an NGC Fraction Collector system (Bio-Rad). The samples were analyzed at a flow rate of 0.5 mL/min using 1×PBS. Detectors were set at 280 nm for the protein and 495 nm for the dye.

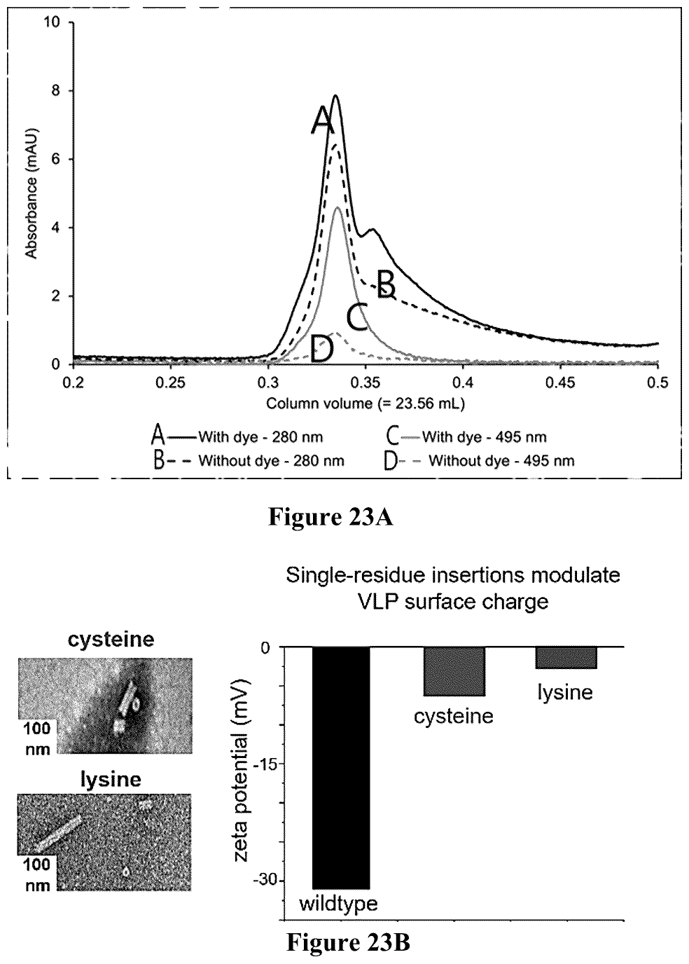

Size exclusion elution profiles of D101R/199C incubated with and without dye showed the proteins eluting between 0.3 and 0.4 column volumes (7 mL to 7.5 mL). As the excess dye was much smaller than the proteins, they could not elute along with the protein; therefore, any signal detected at 495 nm when the VLPs elute confirmed the presence of dye conjugated to the VLPs. The ratio of absorbance peak at 495 nm to absorbance peak at 280 nm was ˜4 times higher in the VLPs with dye than those without dye, thus indicating sufficient dye signal over background and successful conjugation of fluorescein maleimide to the surface-exposed cysteine residues of VLPs (FIGS. 23A and 23B).

### Example 13

### SpyTagged BSMV VLPs

Golden Gate cloning was used to insert SpyTag peptides at the surface-exposed C-terminus of BSMV-D101R VLPs. A SpyTag is a short, unfolded peptide (13 amino acids) that can be genetically fused to exposed positions in target proteins (e.g., a SpyCatcher). Using the tag/catcher pair, bioconjugation can be achieved between two recombinant proteins that would otherwise be restrictive or otherwise impossible with direct genetic fusion. This technology has been used, among other applications, to create covalently stabilized multi-protein complexes and to label proteins.

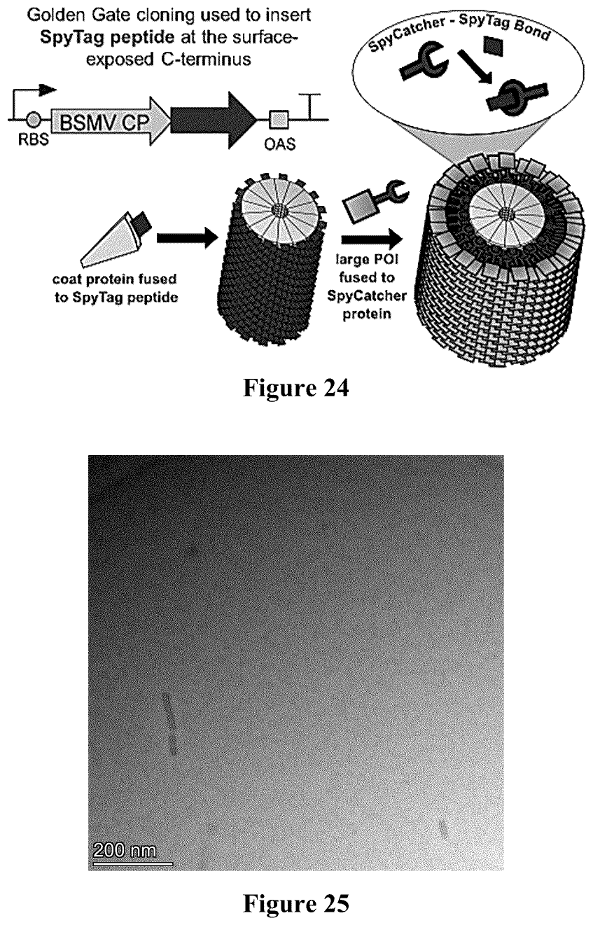

FIG. 24 illustrates the protocol used. FIG. 25 is a TEM image supporting that the BSMV VLP assembly tolerated the SpyTag peptide fusion. Accordingly, the BSMV VLPs hereof can be functionalized and conjugated to diverse peptides/proteins of interest.

While various embodiments of nanoparticles, systems, and methods hereof have been described in considerable detail, the embodiments are merely offered by way of non-limiting examples. Many variations and modifications of the embodiments described herein will be apparent to one of ordinary skill in the art in light of the disclosure. It will therefore be understood by those skilled in the art that various changes and modifications may be made, and equivalents may be substituted for elements thereof, without departing from the scope of the disclosure. Indeed, this disclosure is not intended to be exhaustive or to limiting. The scope of the disclosure is to he defined by the appended claims, and by their equivalents.

Further, in describing representative embodiments, the disclosure may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present disclosure.

It is therefore intended that this description and the appended claims will encompass, all modifications and changes apparent to those of ordinary skill in the art based on this disclosure.

1. A virus-like particle (VLP) comprising one or both of:
(a) an origin of self-assembly (OAS) operatively linked with a Barley stripe mosaic virus coat protein (BSMV-CP) wherein a portion of the nucleic acid sequence that encodes the OAS comprises SEQ ID NO: 11 or a functional equivalent thereof, and
(b) at least one site-directed mutation on the BSMV-CP at residue E62, D101, or both residues E62 and D101, wherein each site-directed mutation independently comprises a neutral or positive amino acid;
wherein the VLP is stable at a pH of at or between about 4 to about 9.

2. The VLP of claim 1, wherein the site-directed mutation at residue E62, D101, or both residues E62 and D101 on the BSMV-CP each independently comprises glutamine, asparagine, arginine, or lysine.

3. The VLP of claim 1, wherein the site-directed mutation at residue E62 comprises SEQ ID NO: 9 or a functional variant thereof, the site-directed mutation at residue D101 comprises SEQ ID NO: 5 or 6, or a functional variant thereof, or the site-directed mutation at both residues E62 and D101 comprises SEQ ID NO: 10 or a functional variant thereof.

4. The VLP of claim 1 comprising a rod length of at or between about 75 nm to about 150 nm.

5. The VLP of claim 1 comprising surface-exposed C-termini, the surface-exposed C-termini comprising a site-directed insertion of a natural amino acid residue or a SpyTag.

6. The VLP of claim 5, wherein the natural amino acid residue comprises a lysine, a histidine, or a cysteine residue.

7. The VLP of claim 5, wherein the surface exposed C-termini is functionalized with a ligand.

8. The VLP of claim 7, wherein the ligand comprises a fluorescent label, an amide, a reactive electrophile, a peptide, a polymer, a small molecule, a metal, or a protein.

9. The VLP of claim 8, wherein the fluorescent label comprises fluorescamine or fluorescein malemide.

10. A method of manufacturing a nanoparticle biotemplate comprising the steps of:
introducing into an isolated host a nucleic acid sequence that encodes a Barley stripe mosaic virus coat protein (BSMV-CP) and one or both of:
(a) an origin of self-assembly (OAS) operatively linked with the BSMV-CP wherein a portion of the nucleic acid sequence that encodes the OAS comprises SEQ ID NO: 11 or a functional equivalent thereof, and
(b) at least one site-directed mutation on the BSMV-CP at residue E62, D101, or both residues E62 and D101, wherein each site-directed mutation independently comprises a neutral or positive amino acid;
expressing the nucleic acid sequence in a microbial expression system to produce self-assembled BSMV viral-like particles (BSMV VLPs); and
isolating the BSMV VLPs from the microbial expression system.

11. The method of claim 10, wherein the nucleic acid sequence encodes a site-directed mutation at residue E62, D101, or both residues E62 and D101 on the BSMV-CP, the site-directed mutation at residue E62, D101, or both residues E62 and D101 on the BSMV-CP independently selected from the group consisting of glutamine, asparagine, arginine, and lysine.

12. The method of claim 10, wherein the BSMV VLPs are stable at a pH of at or between 4-9.

13. The method of claim 10, wherein the BSMV VLPs comprise a higher average rod length as compared to wildtype BSMV VLPs at a neutral pH.

14. The method of claim 10, wherein the nucleic acid sequence further comprises a site-directed mutation at a C-terminus of the BSMV-CP encoding a natural amino acid residue or a SpyTag for display on a surface of the resulting BSMV VLP.

15. The method of claim 14, wherein the natural amino acid residue comprises a lysine, a histidine, or a cysteine.

16. The method of claim 14, further comprising functionalizing the surface of the BSMV VLP with one or more ligands.

17. The method of claim 10, wherein the step of expressing the nucleic acid sequence further comprises:
constructing a plasmid or an expression vector comprising the nucleic acid sequence; and
transforming the plasmid or expression vector into the host;
wherein the host is Escherichia coli and the step of expressing the nucleic acid sequence is performed at a pH at or between 4-9.

18. The method of claim 17, wherein the step of expressing the nucleic acid sequence is performed at a pH of about 4 or about 9.

19. The method of claim 10, wherein the BSMV-CP comprises the BSMV-CP fused with a linker region and the at least one site-directed mutation at residue E62, D101, or both residues E62 and D101 on the BSMV-CP.

20. The method of claim 10, further comprising the step of selecting a length of the linker region based on a desired length in the resulting BSMV VLPs.

21. The method of claim 10, further comprising the step of synthesizing one or more nanoparticles using the resulting VLPs.
